(19) United States
(12) Patent Application Publication
(10) Pub. No.: US 2026/0356937 A1
(43) Pub. Date: Nov. 12, 2026

(54) ANTI-ILT4 ANTIBODY AND PHARMACEUTICAL USE THEREOF

(71) Applicants: Jiangsu Hengrui Pharmaceuticals Co., Ltd., Lianyungang, Jiangsu (CN); Shanghai Hengrui Pharmaceutical Co., Ltd., Shanghai (CN)

(72) Inventors: Xin Ye, Shanghai (CN); Le Sun, Shanghai (CN); Xinsheng Jin, Shanghai (CN); Hua Ying, Shanghai (CN)

(21) Appl. No.: 19/109,554

(22) PCT Filed: Sep. 8, 2023
(86) PCT No.: PCT/CN2023/117653

§ 371 Date: Mar. 6, 2025

(30) Foreign Application Priority Data
Sep. 8, 2022 (CN) 202211093055.2

(51) Int. Cl. C07K 16/28; A61K 39/00; A61P 35/00

(52) U.S. Cl. CPC C07K 16/2803; A61P 35/00; C07K 16/2818; A61K 2039/505; C07K 2317/24; C07K 2317/92

(57) ABSTRACT

The present invention provides an anti-ILT4 antibody and pharmaceutical use thereof.

### CROSS-REFERENCE TO RELATED APPLICATIONS

The present application is a U.S. National Stage entry of International Application No. PCT/CN2023/117653 filed on Sep. 8, 2023, which claims priority to Chinese Patent Application No. 202211093055.2 filed on Sep. 8, 2022. The contents of those applications are incorporated herein by reference in their entireties.

### SEQUENCE LISTING

This application incorporates by reference the material in the ST.26 XML file titled CHENG-78_723081CPUS_Sequence-listing, which was created on Mar. 7, 2025 and is 375,038 bytes.

### TECHNICAL FIELD

The present disclosure pertains to the field of biotechnology. Specifically, the present disclosure relates to an anti-ILT4 antibody and use thereof.

### BACKGROUND

The statements herein merely provide background information related to the present disclosure and may not necessarily constitute the prior art.

Immune checkpoints are molecules expressed on immune cells that can regulate the extent of the immune response. They play a crucial role in preventing autoimmune diseases. When the human body's immune function is stimulated, it becomes activated but does not become overly activated, thanks to immune checkpoints that act as a “brake” in the activation of the immune system to keep the activation within the normal range. Tumor cells can overexpress some immune checkpoint ligands to activate them, thereby inhibiting immune cell functions and evading immune surveillance. Common targeted therapies against T-cell immune checkpoints such as CTLA-4 and PD-1 have achieved significant success. However, due to compensatory mechanisms involving other immune checkpoints, these inhibitors also suffer from low efficiency or resistance. There is still a clinical need to develop other potential immune checkpoint targets.

Immunoglobulin-like transcript 4 (ILT4, also known as LILRB2) belongs to the LILR (leukocyte immunoglobulin-like receptor) family, which has 11 members in total. These members are classified into three categories based on whether their intracellular domains contain ITAM or ITIM. ILT4 is an inhibitory LILRB and is considered to function as an immune checkpoint. ILT4 is expressed on myeloid-derived immune cells, such as macrophages, dendritic cells, and granulocytes. It plays a physiological role in binding to MHC-I molecules to regulate immune balance and homeostasis. In tumor biology, the expression level of ILT4 is positively correlated with the number of myeloid-derived suppressor cells (MDSCs), which are a major cause of resistance to CTLA-4 and PD-1 antibodies. In addition, one of ILT4's ligands, HLA-G (non-classical MHC-I), is reported to be widely expressed in various tumor tissues and is considered an important mechanism for immune evasion of tumor cells. Targeting ILT4 can induce the transformation of macrophages to the pro-inflammatory M1 phenotype, enhance antigen presentation by APCs, and ultimately activate T cells. Meanwhile, ILT4 antibodies can alter the tumor microenvironment to reduce the suppression of T cells by MDSCs.

At present, Merck's MK-4830 (WO2018187518A1) and Jounce's JTX-8064 (WO2019126514A2) have entered phase 2 and phase 1 clinical trials, respectively. According to clinical data released by Merck, the combination of MK-4830 and the PD-1 antibody Keytruda achieved an ORR of 24% in 34 patients with metastatic or advanced solid tumors. Among 11 patients who had experienced disease progression after PD-1 targeted therapy, 5 patients (45%) were in remission, indicating that the ILT4 antibody has a certain potential to overcome resistance to PD-1 targeted therapy. In addition, the adverse reactions observed with the combination of MK-4830 and Keytruda were similar to those observed with Keytruda alone, suggesting that the ILT4 antibody does not raise significant safety concerns.

ILT4 plays an immune negative regulatory role. Inhibitory antibodies targeting it can effectively activate immune cells and can therefore be used as a beneficial complement to existing immune checkpoint therapies.

### SUMMARY

The present disclosure provides an anti-ILT4 antibody comprising a heavy chain variable region and a light chain variable region, wherein the heavy chain variable region comprises a HCDR1, a HCDR2, and a HCDR3, and the light chain variable region comprises a LCDR1, a LCDR2, and a LCDR3, wherein:
a. the HCDR1, the HCDR2, and the HCDR3 of the heavy chain variable region comprise amino acid sequences of a HCDR1, a HCDR2, and a HCDR3, respectively, in SEQ ID NO: 184 or 185, and the LCDR1, the LCDR2, and the LCDR3 of the light chain variable region comprise amino acid sequences of a LCDR1, a LCDR2, and a LCDR3, respectively, in SEQ ID NO: 187 or 186; or the HCDR1, the HCDR2, and the HCDR3 of the heavy chain variable region comprise amino acid sequences of a HCDR1, a HCDR2, and a HCDR3, respectively, in SEQ ID NO: 32, and the LCDR1, the LCDR2, and the LCDR3 of the light chain variable region comprise amino acid sequences of a LCDR1, a LCDR2, and a LCDR3, respectively, in SEQ ID NO: 33; or
b. the HCDR1, the HCDR2, and the HCDR3 of the heavy chain variable region comprise amino acid sequences of a HCDR1, a HCDR2, and a HCDR3, respectively, in any one of SEQ ID NO: 211, 205, 206, 207, 208, 209, or 210, and the LCDR1, the LCDR2, and the LCDR3 of the light chain variable region comprise amino acid sequences of a LCDR1, a LCDR2, and a LCDR3, respectively, in any one of SEQ ID NO: 212, 213, or 214; or the HCDR1, the HCDR2, and the HCDR3 of the heavy chain variable region comprise amino acid sequences of a HCDR1, a HCDR2, and a HCDR3, respectively, in SEQ ID NO: 40, and the LCDR1, the LCDR2, and the LCDR3 of the light chain variable region comprise amino acid sequences of a LCDR1, a LCDR2, and a LCDR3, respectively, in SEQ ID NO: 41; or
c. the HCDR1, the HCDR2, and the HCDR3 of the heavy chain variable region comprise amino acid sequences of a HCDR1, a HCDR2, and a HCDR3, respectively, in any one of SEQ ID NO: 181, 176, 177, 178, 179, or 180, and the LCDR1, the LCDR2, and the LCDR3 of the light chain variable region comprise amino acid sequences of a LCDR1, a LCDR2, and a LCDR3, respectively, in SEQ ID NO: 183 or 182; or the HCDR1, the HCDR2, and the HCDR3 of the heavy chain variable region comprise amino acid sequences of a HCDR1, a HCDR2, and a HCDR3, respectively, in SEQ ID NO: 28, and the LCDR1, the LCDR2, and the LCDR3 of the light chain variable region comprise amino acid sequences of a LCDR1, a LCDR2, and a LCDR3, respectively, in SEQ ID NO: 29; or
d. the HCDR1, the HCDR2, and the HCDR3 of the heavy chain variable region comprise amino acid sequences of a HCDR1, a HCDR2, and a HCDR3, respectively, in any one of SEQ ID NO: 193, 190, 191, or 192, and the LCDR1, the LCDR2, and the LCDR3 of the light chain variable region comprise amino acid sequences of a LCDR1, a LCDR2, and a LCDR3, respectively, in any one of SEQ ID NO: 194, 195, or 196; or the HCDR1, the HCDR2, and the HCDR3 of the heavy chain variable region comprise amino acid sequences of a HCDR1, a HCDR2, and a HCDR3, respectively, in SEQ ID NO: 10, and the LCDR1, the LCDR2, and the LCDR3 of the light chain variable region comprise amino acid sequences of a LCDR1, a LCDR2, and a LCDR3, respectively, in SEQ ID NO: 11; or
e. the HCDR1, the HCDR2, and the HCDR3 of the heavy chain variable region comprise amino acid sequences of a HCDR1, a HCDR2, and a HCDR3, respectively, in SEQ ID NO: 163, and the LCDR1, the LCDR2, and the LCDR3 of the light chain variable region SEQ ID NO: 225 or 164; or
f. the HCDR1, the HCDR2, and the HCDR3 of the heavy chain variable region comprise amino acid sequences of a HCDR1, a HCDR2, and a HCDR3, respectively, in SEQ ID NO: 197 or 198, and the LCDR1, the LCDR2, and the LCDR3 of the light chain variable region comprise amino acid sequences of a LCDR1, a LCDR2, and a LCDR3, respectively, in SEQ ID NO: 199 or 200; or the HCDR1, the HCDR2, and the HCDR3 of the heavy chain variable region comprise amino acid sequences of a HCDR1, a HCDR2, and a HCDR3, respectively, in SEQ ID NO: 36, and the LCDR1, the LCDR2, and the LCDR3 of the light chain variable region comprise amino acid sequences of a LCDR1, a LCDR2, and a LCDR3, respectively, in SEQ ID NO: 37; or
g. the HCDR1, the HCDR2, and the HCDR3 of the heavy chain variable region comprise amino acid sequences of a HCDR1, a HCDR2, and a HCDR3, respectively, in any one of SEQ ID NO: 217, 218, 219, 220, or 221, and the LCDR1, the LCDR2, and the LCDR3 of the light chain variable region comprise amino acid sequences of a LCDR1, a LCDR2, and a LCDR3, respectively, in SEQ ID NO: 222 or 223; or the HCDR1, the HCDR2, and the HCDR3 of the heavy chain variable region comprise amino acid sequences of a HCDR1, a HCDR2, and a HCDR3, respectively, in SEQ ID NO: 42, and the LCDR1, the LCDR2, and the LCDR3 of the light chain variable region comprise amino acid sequences of a LCDR1, a LCDR2, and a LCDR3, respectively, in SEQ ID NO: 43; or
h. the HCDR1, the HCDR2, and the HCDR3 of the heavy chain variable region comprise amino acid sequences of a HCDR1, a HCDR2, and a HCDR3, respectively, in SEQ ID NO: 34, and the LCDR1, the LCDR2, and the LCDR3 of the light chain variable region comprise amino acid sequences of a LCDR1, a LCDR2, and a LCDR3, respectively, in SEQ ID NO: 35; or
i. the HCDR1, the HCDR2, and the HCDR3 of the heavy chain variable region comprise amino acid sequences of a HCDR1, a HCDR2, and a HCDR3, respectively, in SEQ ID NO: 18, and the LCDR1, the LCDR2, and the LCDR3 of the light chain variable region comprise amino acid sequences of a LCDR1, a LCDR2, and a LCDR3, respectively, in SEQ ID NO: 19; or
j. the HCDR1, the HCDR2, and the HCDR3 of the heavy chain variable region comprise amino acid sequences of a HCDR1, a HCDR2, and a HCDR3, respectively, in SEQ ID NO: 20, and the LCDR1, the LCDR2, and the LCDR3 of the light chain variable region SEQ ID NO: 21; or
k. the HCDR1, the HCDR2, and the HCDR3 of the heavy chain variable region comprise amino acid sequences of a HCDR1, a HCDR2, and a HCDR3, respectively, in SEQ ID NO: 24, and the LCDR1, the LCDR2, and the LCDR3 of the light chain variable region comprise amino acid sequences of a LCDR1, a LCDR2, and a LCDR3, respectively, in SEQ ID NO: 25; or
l. the HCDR1, the HCDR2, and the HCDR3 of the heavy chain variable region comprise amino acid sequences of a HCDR1, a HCDR2, and a HCDR3, respectively, in SEQ ID NO: 44, and the LCDR1, the LCDR2, and the LCDR3 of the light chain variable region comprise amino acid sequences of a LCDR1, a LCDR2, and a LCDR3, respectively, in SEQ ID NO: 45; or
m. the HCDR1, the HCDR2, and the HCDR3 of the heavy chain variable region comprise amino acid sequences of a HCDR1, a HCDR2, and a HCDR3, respectively, in SEQ ID NO: 46, and the LCDR1, the LCDR2, and the LCDR3 of the light chain variable region comprise amino acid sequences of a LCDR1, a LCDR2, and a LCDR3, respectively, in SEQ ID NO: 47; or
n. the HCDR1, the HCDR2, and the HCDR3 of the heavy chain variable region comprise amino acid sequences of a HCDR1, a HCDR2, and a HCDR3, respectively, in SEQ ID NO: 48, and the LCDR1, the LCDR2, and the LCDR3 of the light chain variable region comprise amino acid sequences of a LCDR1, a LCDR2, and a LCDR3, respectively, in SEQ ID NO: 49; or
o. the HCDR1, the HCDR2, and the HCDR3 of the heavy chain variable region comprise amino acid sequences of a HCDR1, a HCDR2, and a HCDR3, respectively, in SEQ ID NO: 50, and the LCDR1, the LCDR2, and the LCDR3 of the light chain variable region comprise amino acid sequences of a LCDR1, a LCDR2, and a LCDR3, respectively, in SEQ ID NO: 51; or
p. the HCDR1, the HCDR2, and the HCDR3 of the heavy chain variable region comprise amino acid sequences of a HCDR1, a HCDR2, and a HCDR3, respectively, in SEQ ID NO: 4, and the LCDR1, the LCDR2, and the LCDR3 of the light chain variable region comprise amino acid sequences of a LCDR1, a LCDR2, and a LCDR3, respectively, in SEQ ID NO: 5; or
q. the HCDR1, the HCDR2, and the HCDR3 of the heavy chain variable region comprise amino acid sequences of a HCDR1, a HCDR2, and a HCDR3, respectively, in SEQ ID NO: 6, and the LCDR1, the LCDR2, and the LCDR3 of the light chain variable region comprise amino acid sequences of a LCDR1, a LCDR2, and a LCDR3, respectively, in SEQ ID NO: 7; or
r. the HCDR1, the HCDR2, and the HCDR3 of the heavy chain variable region comprise amino acid sequences of a HCDR1, a HCDR2, and a HCDR3, respectively, in SEQ ID NO: 8, and the LCDR1, the LCDR2, and the LCDR3 of the light chain variable region comprise amino acid sequences of a LCDR1, a LCDR2, and a LCDR3, respectively, in SEQ ID NO: 9; or
s. the HCDR1, the HCDR2, and the HCDR3 of the heavy chain variable region comprise amino acid sequences of a HCDR1, a HCDR2, and a HCDR3, respectively, in SEQ ID NO: 12, and the LCDR1, the LCDR2, and the LCDR3 of the light chain variable region comprise amino acid sequences of a LCDR1, a LCDR2, and a LCDR3, respectively, in SEQ ID NO: 13; or
t. the HCDR1, the HCDR2, and the HCDR3 of the heavy chain variable region comprise amino acid sequences of a HCDR1, a HCDR2, and a HCDR3, respectively, in SEQ ID NO: 14, and the LCDR1, the LCDR2, and the LCDR3 of the light chain variable region comprise amino acid sequences of a LCDR1, a LCDR2, and a LCDR3, respectively, in SEQ ID NO: 15; or
u. the HCDR1, the HCDR2, and the HCDR3 of the heavy chain variable region comprise amino acid sequences of a HCDR1, a HCDR2, and a HCDR3, respectively, in SEQ ID NO: 16, and the LCDR1, the LCDR2, and the LCDR3 of the light chain variable region comprise amino acid sequences of a LCDR1, a LCDR2, and a LCDR3, respectively, in SEQ ID NO: 17; or
v. the HCDR1, the HCDR2, and the HCDR3 of the heavy chain variable region comprise amino acid sequences of a HCDR1, a HCDR2, and a HCDR3, respectively, in SEQ ID NO: 22, and the LCDR1, the LCDR2, and the LCDR3 of the light chain variable region comprise amino acid sequences of a LCDR1, a LCDR2, and a LCDR3, respectively, in SEQ ID NO: 23; or
w. the HCDR1, the HCDR2, and the HCDR3 of the heavy chain variable region comprise amino acid sequences of a HCDR1, a HCDR2, and a HCDR3, respectively, in SEQ ID NO: 26, and the LCDR1, the LCDR2, and the LCDR3 of the light chain variable region comprise amino acid sequences of a LCDR1, a LCDR2, and a LCDR3, respectively, in SEQ ID NO: 27; or
x. the HCDR1, the HCDR2, and the HCDR3 of the heavy chain variable region comprise amino acid sequences of a HCDR1, a HCDR2, and a HCDR3, respectively, in SEQ ID NO: 30, and the LCDR1, the LCDR2, and the LCDR3 of the light chain variable region comprise amino acid sequences of a LCDR1, a LCDR2, and a LCDR3, respectively, in SEQ ID NO: 31; or
y. the HCDR1, the HCDR2, and the HCDR3 of the heavy chain variable region comprise amino acid sequences of a HCDR1, a HCDR2, and a HCDR3, respectively, in SEQ ID NO: 38, and the LCDR1, the LCDR2, and the LCDR3 of the light chain variable region comprise amino acid sequences of a LCDR1, a LCDR2, and a LCDR3, respectively, in SEQ ID NO: 39.

In some embodiments, provided is the aforementioned anti-ILT4 antibody, wherein:
a. the HCDR1, the HCDR2, and the HCDR3 of the heavy chain variable region comprise amino acid sequences of a HCDR1, a HCDR2, and a HCDR3, respectively, in SEQ ID NO: 184 or 185, and the LCDR1, the LCDR2, and the LCDR3 of the light chain variable region comprise amino acid sequences of a LCDR1, a LCDR2, and a LCDR3, respectively, in SEQ ID NO: 187 or 186; or the HCDR1, the HCDR2, and the HCDR3 of the heavy chain variable region comprise amino acid sequences of a HCDR1, a HCDR2, and a HCDR3, respectively, in SEQ ID NO: 32, and the LCDR1, the LCDR2, and the LCDR3 of the light chain variable region comprise amino acid sequences of a LCDR1, a LCDR2, and a LCDR3, respectively, in SEQ ID NO: 33; or
b. the HCDR1, the HCDR2, and the HCDR3 of the heavy chain variable region comprise amino acid sequences of a HCDR1, a HCDR2, and a HCDR3, respectively, in any one of SEQ ID NO: 211, 205, 206, 207, 208, 209, or 210, and the LCDR1, the LCDR2, and the LCDR3 of the light chain variable region comprise amino acid sequences of a LCDR1, a LCDR2, and a LCDR3, respectively, in any one of SEQ ID NO: 212, 213, or 214; or the HCDR1, the HCDR2, and the HCDR3 of the heavy chain variable region comprise amino acid sequences of a HCDR1, a HCDR2, and a HCDR3, respectively, in SEQ ID NO: 40, and the LCDR1, the LCDR2, and the LCDR3 of the light chain variable region comprise amino acid sequences of a LCDR1, a LCDR2, and a LCDR3, respectively, in SEQ ID NO: 41; or
c. the HCDR1, the HCDR2, and the HCDR3 of the heavy chain variable region comprise amino acid sequences of a HCDR1, a HCDR2, and a HCDR3, respectively, in any one of SEQ ID NO: 181, 176, 177, 178, 179, or 180, and the LCDR1, the LCDR2, and the LCDR3 of the light chain variable region comprise amino acid sequences of a LCDR1, a LCDR2, and a LCDR3, respectively, in SEQ ID NO: 183 or 182; or the HCDR1, the HCDR2, and the HCDR3 of the heavy chain variable region comprise amino acid sequences of a HCDR1, a HCDR2, and a HCDR3, respectively, in SEQ ID NO: 28, and the LCDR1, the LCDR2, and the LCDR3 of the light chain variable region comprise amino acid sequences of a LCDR1, a LCDR2, and a LCDR3, respectively, in SEQ ID NO: 29; or
d. the HCDR1, the HCDR2, and the HCDR3 of the heavy chain variable region comprise amino acid sequences of a HCDR1, a HCDR2, and a HCDR3, respectively, in any one of SEQ ID NO: 193, 190, 191, or 192, and the LCDR1, the LCDR2, and the LCDR3 of the light chain variable region comprise amino acid sequences of a LCDR1, a LCDR2, and a LCDR3, respectively, in any one of SEQ ID NO: 194, 195, or 196; or the HCDR1, the HCDR2, and the HCDR3 of the heavy chain variable region comprise amino acid sequences of a HCDR1, a HCDR2, and a HCDR3, respectively, in SEQ ID NO: 10, and the LCDR1, the LCDR2, and the LCDR3 of the light chain variable region comprise amino acid sequences of a LCDR1, a LCDR2, and a LCDR3, respectively, in SEQ ID NO: 11; or
e. the HCDR1, the HCDR2, and the HCDR3 of the heavy chain variable region comprise amino acid sequences of a HCDR1, a HCDR2, and a HCDR3, respectively, in SEQ ID NO: 163, and the LCDR1, the LCDR2, and the LCDR3 of the light chain variable region comprise amino acid sequences of a LCDR1, a LCDR2, and a LCDR3, respectively, in SEQ ID NO: 225 or 164; or
f. the HCDR1, the HCDR2, and the HCDR3 of the heavy chain variable region comprise amino acid sequences of a HCDR1, a HCDR2, and a HCDR3, respectively, in SEQ ID NO: 197 or 198, and the LCDR1, the LCDR2, and the LCDR3 of the light chain variable region comprise amino acid sequences of a LCDR1, a LCDR2, and a LCDR3, respectively, in SEQ ID NO: 199 or 200; or the HCDR1, the HCDR2, and the HCDR3 of the heavy chain variable region comprise amino acid sequences of a HCDR1, a HCDR2, and a HCDR3, respectively, in SEQ ID NO: 36, and the LCDR1, the LCDR2, and the LCDR3 of the light chain variable region comprise amino acid sequences of a LCDR1, a LCDR2, and a LCDR3, respectively, in SEQ ID NO: 37; or
g. the HCDR1, the HCDR2, and the HCDR3 of the heavy chain variable region comprise amino acid sequences of a HCDR1, a HCDR2, and a HCDR3, respectively, in any one of SEQ ID NO: 217, 218, 219, 220, or 221, and the LCDR1, the LCDR2, and the LCDR3 of the light chain variable region comprise amino acid sequences of a LCDR1, a LCDR2, and a LCDR3, respectively, in SEQ ID NO: 222 or 223; or the HCDR1, the HCDR2, and the HCDR3 of the heavy chain variable region comprise amino acid sequences of a HCDR1, a HCDR2, and a HCDR3, respectively, in SEQ ID NO: 42, and the LCDR1, the LCDR2, and the LCDR3 of the light chain variable region comprise amino acid sequences of a LCDR1, a LCDR2, and a LCDR3, respectively, in SEQ ID NO: 43; or
h. the HCDR1, the HCDR2, and the HCDR3 of the heavy chain variable region comprise amino acid sequences of a HCDR1, a HCDR2, and a HCDR3, respectively, in SEQ ID NO: 34, and the LCDR1, the LCDR2, and the LCDR3 of the light chain variable region comprise amino acid sequences of a LCDR1, a LCDR2, and a LCDR3, respectively, in SEQ ID NO: 35; or
i. the HCDR1, the HCDR2, and the HCDR3 of the heavy chain variable region comprise amino acid sequences of a HCDR1, a HCDR2, and a HCDR3, respectively, in SEQ ID NO: 18, and the LCDR1, the LCDR2, and the LCDR3 of the light chain variable region comprise amino acid sequences of a LCDR1, a LCDR2, and a LCDR3, respectively, in SEQ ID NO: 19; or
j. the HCDR1, the HCDR2, and the HCDR3 of the heavy chain variable region comprise amino acid sequences of a HCDR1, a HCDR2, and a HCDR3, respectively, in SEQ ID NO: 20, and the LCDR1, the LCDR2, and the LCDR3 of the light chain variable region comprise amino acid sequences of a LCDR1, a LCDR2, and a LCDR3, respectively, in SEQ ID NO: 21; or
k. the HCDR1, the HCDR2, and the HCDR3 of the heavy chain variable region comprise amino acid sequences of a HCDR1, a HCDR2, and a HCDR3, respectively, in SEQ ID NO: 24, and the LCDR1, the LCDR2, and the LCDR3 of the light chain variable region comprise amino acid sequences of a LCDR1, a LCDR2, and a LCDR3, respectively, in SEQ ID NO: 25; or
l. the HCDR1, the HCDR2, and the HCDR3 of the heavy chain variable region comprise amino acid sequences of a HCDR1, a HCDR2, and a HCDR3, respectively, in SEQ ID NO: 44, and the LCDR1, the LCDR2, and the LCDR3 of the light chain variable region comprise amino acid sequences of a LCDR1, a LCDR2, and a LCDR3, respectively, in SEQ ID NO: 45; or
m. the HCDR1, the HCDR2, and the HCDR3 of the heavy chain variable region comprise amino acid sequences of a HCDR1, a HCDR2, and a HCDR3, respectively, in SEQ ID NO: 46, and the LCDR1, the LCDR2, and the LCDR3 of the light chain variable region comprise amino acid sequences of a LCDR1, a LCDR2, and a LCDR3, respectively, in SEQ ID NO: 47; or
n. the HCDR1, the HCDR2, and the HCDR3 of the heavy chain variable region comprise amino acid sequences of a HCDR1, a HCDR2, and a HCDR3, respectively, in SEQ ID NO: 48, and the LCDR1, the LCDR2, and the LCDR3 of the light chain variable region comprise amino acid sequences of a LCDR1, a LCDR2, and a LCDR3, respectively, in SEQ ID NO: 49; or
o. the HCDR1, the HCDR2, and the HCDR3 of the heavy chain variable region comprise amino acid sequences of a HCDR1, a HCDR2, and a HCDR3, respectively, in SEQ ID NO: 50, and the LCDR1, the LCDR2, and the LCDR3 of the light chain variable region comprise amino acid sequences of a LCDR1, a LCDR2, and a LCDR3, respectively, in SEQ ID NO: 51.

In some embodiments, provided is the aforementioned anti-ILT4 antibody, wherein:
a. the HCDR1, the HCDR2, and the HCDR3 of the heavy chain variable region comprise amino acid sequences of a HCDR1, a HCDR2, and a HCDR3, respectively, in SEQ ID NO: 184, and the LCDR1, the LCDR2, and the LCDR3 of the light chain variable region comprise amino acid sequences of a LCDR1, a LCDR2, and a LCDR3, respectively, in SEQ ID NO: 187; or
b. the HCDR1, the HCDR2, and the HCDR3 of the heavy chain variable region comprise amino acid sequences of a HCDR1, a HCDR2, and a HCDR3, respectively, in SEQ ID NO: 211, and the LCDR1, the LCDR2, and the LCDR3 of the light chain variable region comprise amino acid sequences of a LCDR1, a LCDR2, and a LCDR3, respectively, in SEQ ID NO: 212; or
c. the HCDR1, the HCDR2, and the HCDR3 of the heavy chain variable region comprise amino acid sequences of a HCDR1, a HCDR2, and a HCDR3, respectively, in SEQ ID NO: 181, and the LCDR1, the LCDR2, and the LCDR3 of the light chain variable region comprise amino acid sequences of a LCDR1, a LCDR2, and a LCDR3, respectively, in SEQ ID NO: 183; or
d. the HCDR1, the HCDR2, and the HCDR3 of the heavy chain variable region comprise amino acid sequences of a HCDR1, a HCDR2, and a HCDR3, respectively, in SEQ ID NO: 193, and the LCDR1, the LCDR2, and the LCDR3 of the light chain variable region comprise amino acid sequences of a LCDR1, a LCDR2, and a LCDR3, respectively, in SEQ ID NO: 194; or
e. the HCDR1, the HCDR2, and the HCDR3 of the heavy chain variable region comprise amino acid sequences of a HCDR1, a HCDR2, and a HCDR3, respectively, in SEQ ID NO: 163, and the LCDR1, the LCDR2, and the LCDR3 of the light chain variable region comprise amino acid sequences of a LCDR1, a LCDR2, and a LCDR3, respectively, in SEQ ID NO: 225; or
f. the HCDR1, the HCDR2, and the HCDR3 of the heavy chain variable region comprise amino acid sequences of a HCDR1, a HCDR2, and a HCDR3, respectively, in SEQ ID NO: 197, and the LCDR1, the LCDR2, and the LCDR3 of the light chain variable region comprise amino acid sequences of a LCDR1, a LCDR2, and a LCDR3, respectively, in SEQ ID NO: 199; or
g. the HCDR1, the HCDR2, and the HCDR3 of the heavy chain variable region comprise amino acid sequences of a HCDR1, a HCDR2, and a HCDR3, respectively, in SEQ ID NO: 217, and the LCDR1, the LCDR2, and the LCDR3 of the light chain variable region comprise amino acid sequences of a LCDR1, a LCDR2, and a LCDR3, respectively, in SEQ ID NO: 222.

In some embodiments, provided is the aforementioned anti-ILT4 antibody, wherein:
a. the HCDR1, the HCDR2, and the HCDR3 of the heavy chain variable region comprise amino acid sequences of a HCDR1, a HCDR2, and a HCDR3, respectively, in SEQ ID NO: 184, and the LCDR1, the LCDR2, and the LCDR3 of the light chain variable region comprise amino acid sequences of a LCDR1, a LCDR2, and a LCDR3, respectively, in SEQ ID NO: 187; or
b. the HCDR1, the HCDR2, and the HCDR3 of the heavy chain variable region comprise amino acid sequences of a HCDR1, a HCDR2, and a HCDR3, respectively, in SEQ ID NO: 211, and the LCDR1, the LCDR2, and the LCDR3 of the light chain variable region comprise amino acid sequences of a LCDR1, a LCDR2, and a LCDR3, respectively, in SEQ ID NO: 212.

In some embodiments, provided is the aforementioned anti-ILT4 antibody, wherein:
p. the HCDR1, the HCDR2, and the HCDR3 of the heavy chain variable region comprise amino acid sequences of a HCDR1, a HCDR2, and a HCDR3, respectively, in SEQ ID NO: 4, and the LCDR1, the LCDR2, and the LCDR3 of the light chain variable region comprise amino acid sequences of a LCDR1, a LCDR2, and a LCDR3, respectively, in SEQ ID NO: 5; or
q. the HCDR1, the HCDR2, and the HCDR3 of the heavy chain variable region comprise amino acid sequences of a HCDR1, a HCDR2, and a HCDR3, respectively, in SEQ ID NO: 6, and the LCDR1, the LCDR2, and the LCDR3 of the light chain variable region comprise amino acid sequences of a LCDR1, a LCDR2, and a LCDR3, respectively, in SEQ ID NO: 7; or
r. the HCDR1, the HCDR2, and the HCDR3 of the heavy chain variable region comprise amino acid sequences of a HCDR1, a HCDR2, and a HCDR3, respectively, in SEQ ID NO: 8, and the LCDR1, the LCDR2, and the LCDR3 of the light chain variable region comprise amino acid sequences of a LCDR1, a LCDR2, and a LCDR3, respectively, in SEQ ID NO: 9; or
s. the HCDR1, the HCDR2, and the HCDR3 of the heavy chain variable region comprise amino acid sequences of a HCDR1, a HCDR2, and a HCDR3, respectively, in SEQ ID NO: 12, and the LCDR1, the LCDR2, and the LCDR3 of the light chain variable region comprise amino acid sequences of a LCDR1, a LCDR2, and a LCDR3, respectively, in SEQ ID NO: 13; or
t. the HCDR1, the HCDR2, and the HCDR3 of the heavy chain variable region comprise amino acid sequences of a HCDR1, a HCDR2, and a HCDR3, respectively, in SEQ ID NO: 14, and the LCDR1, the LCDR2, and the LCDR3 of the light chain variable region comprise amino acid sequences of a LCDR1, a LCDR2, and a LCDR3, respectively, in SEQ ID NO: 15; or
u. the HCDR1, the HCDR2, and the HCDR3 of the heavy chain variable region comprise amino acid sequences of a HCDR1, a HCDR2, and a HCDR3, respectively, in SEQ ID NO: 16, and the LCDR1, the LCDR2, and the LCDR3 of the light chain variable region comprise amino acid sequences of a LCDR1, a LCDR2, and a LCDR3, respectively, in SEQ ID NO: 17; or
v. the HCDR1, the HCDR2, and the HCDR3 of the heavy chain variable region comprise amino acid sequences of a HCDR1, a HCDR2, and a HCDR3, respectively, in SEQ ID NO: 22, and the LCDR1, the LCDR2, and the LCDR3 of the light chain variable region comprise amino acid sequences of a LCDR1, a LCDR2, and a LCDR3, respectively, in SEQ ID NO: 23; or
w. the HCDR1, the HCDR2, and the HCDR3 of the heavy chain variable region comprise amino acid sequences of a HCDR1, a HCDR2, and a HCDR3, respectively, in SEQ ID NO: 26, and the LCDR1, the LCDR2, and the LCDR3 of the light chain variable region SEQ ID NO: 27; or
x. the HCDR1, the HCDR2, and the HCDR3 of the heavy chain variable region comprise amino acid sequences of a HCDR1, a HCDR2, and a HCDR3, respectively, in SEQ ID NO: 30, and the LCDR1, the LCDR2, and the LCDR3 of the light chain variable region comprise amino acid sequences of a LCDR1, a LCDR2, and a LCDR3, respectively, in SEQ ID NO: 31; or
y. the HCDR1, the HCDR2, and the HCDR3 of the heavy chain variable region comprise amino acid sequences of a HCDR1, a HCDR2, and a HCDR3, respectively, in SEQ ID NO: 38, and the LCDR1, the LCDR2, and the LCDR3 of the light chain variable region comprise amino acid sequences of a LCDR1, a LCDR2, and a LCDR3, respectively, in SEQ ID NO: 39.

In some embodiments, provided is the anti-ILT4 antibody according to any one of the above, wherein the HCDR1, the HCDR2, and the HCDR3 of the heavy chain variable region and the LCDR1, the LCDR2, and the LCDR3 of the light chain variable region are defined according to the same numbering scheme selected from the group consisting of Kabat, IMGT, Chothia, AbM, and Contact. In some embodiments, the HCDR1, the HCDR2, and the HCDR3 of the heavy chain variable region and the LCDR1, the LCDR2, and the LCDR3 of the light chain variable region are defined according to the Kabat numbering scheme. In some embodiments, the HCDR1, the HCDR2, and the HCDR3 of the heavy chain variable region and the LCDR1, the LCDR2, and the LCDR3 of the light chain variable region are defined according to the IMGT numbering scheme. In some embodiments, the HCDR1, the HCDR2, and the HCDR3 of the heavy chain variable region and the LCDR1, the LCDR2, and the LCDR3 of the light chain variable region are defined according to the Chothia numbering scheme. In some embodiments, the HCDR1, the HCDR2, and the HCDR3 of the heavy chain variable region and the LCDR1, the LCDR2, and the LCDR3 of the light chain variable region are defined according to the AbM numbering scheme. In some embodiments, the HCDR1, the HCDR2, and the HCDR3 of the heavy chain variable region and the LCDR1, the LCDR2, and the LCDR3 of the light chain variable region are defined according to the Contact numbering scheme.

In some embodiments, provided is the anti-ILT4 antibody according to any one of the above, wherein:
a. in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119; or
b. in the heavy chain variable region, the HCDR1 comprises an amino acid sequence of SEQ ID NO: 135, the HCDR2 comprises an amino acid sequence of any one of SEQ ID NO: 203, 136, 201, 202, or 204, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140; or
c. in the heavy chain variable region, the HCDR1 comprises an amino acid sequence of SEQ ID NO: 111, the HCDR2 comprises an amino acid sequence of any one of SEQ ID NO: 173, 112, 174, or 175, and the HCDR3 comprises an amino acid sequence of SEQ ID NO: 113; and in the light chain variable region, the LCDR1 comprises an amino acid sequence of SEQ ID NO: 114, the LCDR2 comprises an amino acid sequence of SEQ ID NO: 115, and the LCDR3 comprises an amino acid sequence of SEQ ID NO: 116; or
d. in the heavy chain variable region, the HCDR1 comprises an amino acid sequence of SEQ ID NO: 69, the HCDR2 comprises an amino acid sequence of any one of SEQ ID NO: 189, 70, or 188, and the HCDR3 comprises an amino acid sequence of SEQ ID NO: 71; and in the light chain variable region, the LCDR1 comprises an amino acid sequence of SEQ ID NO: 61, the LCDR2 comprises an amino acid sequence of SEQ ID NO: 62, and the LCDR3 comprises an amino acid sequence of SEQ ID NO: 72; or
e. in the heavy chain variable region, the HCDR1 comprises an amino acid sequence of SEQ ID NO: 165, the HCDR2 comprises an amino acid sequence of SEQ ID NO: 166, and the HCDR3 comprises an amino acid sequence of SEQ ID NO: 167; and in the light chain variable region, the LCDR1 comprises an amino acid sequence of SEQ ID NO: 224 or 168, the LCDR2 comprises an amino acid sequence of SEQ ID NO: 169, and the LCDR3 comprises an amino acid sequence of SEQ ID NO: 170; or
f. in the heavy chain variable region, the HCDR1 comprises an amino acid sequence of SEQ ID NO: 105, the HCDR2 comprises an amino acid sequence of SEQ ID NO: 125, and the HCDR3 comprises an amino acid sequence of SEQ ID NO: 126; and in the light chain variable region, the LCDR1 comprises an amino acid sequence of SEQ ID NO: 127, the LCDR2 comprises an amino acid sequence of SEQ ID NO: 109, and the LCDR3 comprises an amino acid sequence of SEQ ID NO: 128; or
g. in the heavy chain variable region, the HCDR1 comprises an amino acid sequence of SEQ ID NO: 141, the HCDR2 comprises an amino acid sequence of SEQ ID NO: 215 or 142, and the HCDR3 comprises an amino acid sequence of SEQ ID NO: 216 or 143; and in the light chain variable region, the LCDR1 comprises an amino acid sequence of SEQ ID NO: 144, the LCDR2 comprises an amino acid sequence of SEQ ID NO: 145, and the LCDR3 comprises an amino acid sequence of SEQ ID NO: 146; or
h. in the heavy chain variable region, the HCDR1 comprises an amino acid sequence of SEQ ID NO: 99, the HCDR2 comprises an amino acid sequence of SEQ ID NO: 122, and the HCDR3 comprises an amino acid sequence of SEQ ID NO: 123; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 124; or
i. in the heavy chain variable region, the HCDR1 comprises an amino acid sequence of SEQ ID NO: 73, the HCDR2 comprises an amino acid sequence of SEQ ID NO: 87, and the HCDR3 comprises an amino acid sequence of SEQ ID NO: 88; and in the light chain variable region, the LCDR1 comprises an amino acid sequence of SEQ ID NO: 61, the LCDR2 comprises an amino acid sequence of SEQ ID NO: 89, and the LCDR3 comprises an amino acid sequence of SEQ ID NO: 90; or
j. in the heavy chain variable region, the HCDR1 comprises an amino acid sequence of SEQ ID NO: 91, the HCDR2 comprises an amino acid sequence of SEQ ID NO: 92, and the HCDR3 comprises an amino acid sequence of SEQ ID NO: 93; and in the light chain variable region, the LCDR1 comprises an amino acid sequence of SEQ ID NO: 85, the LCDR2 comprises an amino acid sequence of SEQ ID NO: 62, and the LCDR3 comprises an amino acid sequence of SEQ ID NO: 94; or
k. in the heavy chain variable region, the HCDR1 comprises an amino acid sequence of SEQ ID NO: 99, the HCDR2 comprises an amino acid sequence of SEQ ID NO: 100, and the HCDR3 comprises an amino acid sequence of SEQ ID NO: 101; and in the light chain variable region, the LCDR1 comprises an amino acid sequence of SEQ ID NO: 102, the LCDR2 comprises an amino acid sequence of SEQ ID NO: 103, and the LCDR3 comprises an amino acid sequence of SEQ ID NO: 104; or
l. in the heavy chain variable region, the HCDR1 comprises an amino acid sequence of SEQ ID NO: 147, the HCDR2 comprises an amino acid sequence of SEQ ID NO: 148, and the HCDR3 comprises an amino acid sequence of SEQ ID NO: 149; and in the light chain variable region, the LCDR1 comprises an amino acid sequence of SEQ ID NO: 150, the LCDR2 comprises an amino acid sequence of SEQ ID NO: 151, and the LCDR3 comprises an amino acid sequence of SEQ ID NO: 152; or
m. in the heavy chain variable region, the HCDR1 comprises an amino acid sequence of SEQ ID NO: 153, the HCDR2 comprises an amino acid sequence of SEQ ID NO: 154, and the HCDR3 comprises an amino acid sequence of SEQ ID NO: 155; and in the light chain variable region, the LCDR1 comprises an amino acid sequence of SEQ ID NO: 114, the LCDR2 comprises an amino acid sequence of SEQ ID NO: 133, and the LCDR3 comprises an amino acid sequence of SEQ ID NO: 119; or
n. in the heavy chain variable region, the HCDR1 comprises an amino acid sequence of SEQ ID NO: 58, the HCDR2 comprises an amino acid sequence of SEQ ID NO: 156, and the HCDR3 comprises an amino acid sequence of SEQ ID NO: 157; and in the light chain variable region, the LCDR1 comprises an amino acid sequence of SEQ ID NO: 158, the LCDR2 comprises an amino acid sequence of SEQ ID NO: 62, and the LCDR3 comprises an amino acid sequence of SEQ ID NO: 159; or
o. in the heavy chain variable region, the HCDR1 comprises an amino acid sequence of SEQ ID NO: 105, the HCDR2 comprises an amino acid sequence of SEQ ID NO: 160, and the HCDR3 comprises an amino acid sequence of SEQ ID NO: 161; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 162; or
p. in the heavy chain variable region, the HCDR1 comprises an amino acid sequence of SEQ ID NO: 52, the HCDR2 comprises an amino acid sequence of SEQ ID NO: 53, and the HCDR3 comprises an amino acid sequence of SEQ ID NO: 54; and in the light chain variable region, the LCDR1 comprises an amino acid sequence of SEQ ID NO: 55, the LCDR2 comprises an amino acid sequence of SEQ ID NO: 56, and the LCDR3 comprises an amino acid sequence of SEQ ID NO: 57; or
q. in the heavy chain variable region, the HCDR1 comprises an amino acid sequence of SEQ ID NO: 58, the HCDR2 comprises an amino acid sequence of SEQ ID NO: 59, and the HCDR3 comprises an amino acid sequence of SEQ ID NO: 60; and in the light chain variable region, the LCDR1 comprises an amino acid sequence of SEQ ID NO: 61, the LCDR2 comprises an amino acid sequence of SEQ ID NO: 62, and the LCDR3 comprises an amino acid sequence of SEQ ID NO: 63; or
r. in the heavy chain variable region, the HCDR1 comprises an amino acid sequence of SEQ ID NO: 64, the HCDR2 comprises an amino acid sequence of SEQ ID NO: 65, and the HCDR3 comprises an amino acid sequence of SEQ ID NO: 66; and in the light chain variable region, the LCDR1 comprises an amino acid sequence of SEQ ID NO: 67, the LCDR2 comprises an amino acid sequence of SEQ ID NO: 56, and the LCDR3 comprises an amino acid sequence of SEQ ID NO: 68; or
s. in the heavy chain variable region, the HCDR1 comprises an amino acid sequence of SEQ ID NO: 73, the HCDR2 comprises an amino acid sequence of SEQ ID NO: 74, and the HCDR3 comprises an amino acid sequence of SEQ ID NO: 75; and in the light chain variable region, the LCDR1 comprises an amino acid sequence of SEQ ID NO: 76, the LCDR2 comprises an amino acid sequence of SEQ ID NO: 62, and the LCDR3 comprises an amino acid sequence of SEQ ID NO: 77; or
t. in the heavy chain variable region, the HCDR1 comprises an amino acid sequence of SEQ ID NO: 78, the HCDR2 comprises an amino acid sequence of SEQ ID NO: 79, and the HCDR3 comprises an amino acid sequence of SEQ ID NO: 80; and in the light chain variable region, the LCDR1 comprises an amino acid sequence of SEQ ID NO: 81, the LCDR2 comprises an amino acid sequence of SEQ ID NO: 62, and the LCDR3 comprises an amino acid sequence of SEQ ID NO: 82; or
u. in the heavy chain variable region, the HCDR1 comprises an amino acid sequence of SEQ ID NO: 52, the HCDR2 comprises an amino acid sequence of SEQ ID NO: 83, and the HCDR3 comprises an amino acid sequence of SEQ ID NO: 84; and in the light chain variable region, the LCDR1 comprises an amino acid sequence of SEQ ID NO: 85, the LCDR2 comprises an amino acid sequence of SEQ ID NO: 62, and the LCDR3 comprises an amino acid sequence of SEQ ID NO: 86; or
v. in the heavy chain variable region, the HCDR1 comprises an amino acid sequence of SEQ ID NO: 95, the HCDR2 comprises an amino acid sequence of SEQ ID NO: 96, and the HCDR3 comprises an amino acid sequence of SEQ ID NO: 97; and in the light chain variable region, the LCDR1 comprises an amino acid sequence of SEQ ID NO: 85, the LCDR2 comprises an amino acid sequence of SEQ ID NO: 98, and the LCDR3 comprises an amino acid sequence of SEQ ID NO: 68; or
w. in the heavy chain variable region, the HCDR1 comprises an amino acid sequence of SEQ ID NO: 105, the HCDR2 comprises an amino acid sequence of SEQ ID NO: 106, and the HCDR3 comprises an amino acid sequence of SEQ ID NO: 107; and in the light chain variable region, the LCDR1 comprises an amino acid sequence of SEQ ID NO: 108, the LCDR2 comprises an amino acid sequence of SEQ ID NO: 109, and the LCDR3 comprises an amino acid sequence of SEQ ID NO: 110; or
x. in the heavy chain variable region, the HCDR1 comprises an amino acid sequence of SEQ ID NO: 105, the HCDR2 comprises an amino acid sequence of SEQ ID NO: 117, and the HCDR3 comprises an amino acid sequence of SEQ ID NO: 118; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119; or
y. in the heavy chain variable region, the HCDR1 comprises an amino acid sequence of SEQ ID NO: 129, the HCDR2 comprises an amino acid sequence of SEQ ID NO: 130, and the HCDR3 comprises an amino acid sequence of SEQ ID NO: 131; and in the light chain variable region, the LCDR1 comprises an amino acid sequence of SEQ ID NO: 132, the LCDR2 comprises an amino acid sequence of SEQ ID NO: 133, and the LCDR3 comprises an amino acid sequence of SEQ ID NO: 134.

In some embodiments, provided is the anti-ILT4 antibody according to any one of the above, wherein:
a. in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119; or
b. in the heavy chain variable region, the HCDR1 comprises an amino acid sequence of SEQ ID NO: 135, the HCDR2 comprises an amino acid sequence of any one of SEQ ID NO: 203, 136, 201, 202, or 204, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140; or
c. in the heavy chain variable region, the HCDR1 comprises an amino acid sequence of SEQ ID NO: 111, the HCDR2 comprises an amino acid sequence of any one of SEQ ID NO: 173, 112, 174, or 175, and the HCDR3 comprises an amino acid sequence of SEQ ID NO: 113; and in the light chain variable region, the LCDR1 comprises an amino acid sequence of SEQ ID NO: 114, the LCDR2 comprises an amino acid sequence of SEQ ID NO: 115, and the LCDR3 comprises an amino acid sequence of SEQ ID NO: 116; or
d. in the heavy chain variable region, the HCDR1 comprises an amino acid sequence of SEQ ID NO: 69, the HCDR2 comprises an amino acid sequence of any one of SEQ ID NO: 189, 70, or 188, and the HCDR3 comprises an amino acid sequence of SEQ ID NO: 71; and in the light chain variable region, the LCDR1 comprises an amino acid sequence of SEQ ID NO: 61, the LCDR2 comprises an amino acid sequence of SEQ ID NO: 62, and the LCDR3 comprises an amino acid sequence of SEQ ID NO: 72; or
e. in the heavy chain variable region, the HCDR1 comprises an amino acid sequence of SEQ ID NO: 165, the HCDR2 comprises an amino acid sequence of SEQ ID NO: 166, and the HCDR3 comprises an amino acid sequence of SEQ ID NO: 167; and in the light chain variable region, the LCDR1 comprises an amino acid sequence of SEQ ID NO: 224 or 168, the LCDR2 comprises an amino acid sequence of SEQ ID NO: 169, and the LCDR3 comprises an amino acid sequence of SEQ ID NO: 170; or
f. in the heavy chain variable region, the HCDR1 comprises an amino acid sequence of SEQ ID NO: 105, the HCDR2 comprises an amino acid sequence of SEQ ID NO: 125, and the HCDR3 comprises an amino acid sequence of SEQ ID NO: 126; and in the light chain variable region, the LCDR1 comprises an amino acid sequence of SEQ ID NO: 127, the LCDR2 comprises an amino acid sequence of SEQ ID NO: 109, and the LCDR3 comprises an amino acid sequence of SEQ ID NO: 128; or
g. in the heavy chain variable region, the HCDR1 comprises an amino acid sequence of SEQ ID NO: 141, the HCDR2 comprises an amino acid sequence of SEQ ID NO: 215 or 142, and the HCDR3 comprises an amino acid sequence of SEQ ID NO: 216 or 143; and in the light chain variable region, the LCDR1 comprises an amino acid sequence of SEQ ID NO: 144, the LCDR2 comprises an amino acid sequence of SEQ ID NO: 145, and the LCDR3 comprises an amino acid sequence of SEQ ID NO: 146; or
h. in the heavy chain variable region, the HCDR1 comprises an amino acid sequence of SEQ ID NO: 99, the HCDR2 comprises an amino acid sequence of SEQ ID NO: 122, and the HCDR3 comprises an amino acid sequence of SEQ ID NO: 123; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 124; or
i. in the heavy chain variable region, the HCDR1 comprises an amino acid sequence of SEQ ID NO: 73, the HCDR2 comprises an amino acid sequence of SEQ ID NO: 87, and the HCDR3 comprises an amino acid sequence of SEQ ID NO: 88; and in the light chain variable region, the LCDR1 comprises an amino acid sequence of SEQ ID NO: 61, the LCDR2 comprises an amino acid sequence of SEQ ID NO: 89, and the LCDR3 comprises an amino acid sequence of SEQ ID NO: 90; or
j. in the heavy chain variable region, the HCDR1 comprises an amino acid sequence of SEQ ID NO: 91, the HCDR2 comprises an amino acid sequence of SEQ ID NO: 92, and the HCDR3 comprises an amino acid sequence of SEQ ID NO: 93; and in the light chain variable region, the LCDR1 comprises an amino acid sequence of SEQ ID NO: 85, the LCDR2 comprises an amino acid sequence of SEQ ID NO: 62, and the LCDR3 comprises an amino acid sequence of SEQ ID NO: 94; or
k. in the heavy chain variable region, the HCDR1 comprises an amino acid sequence of SEQ ID NO: 99, the HCDR2 comprises an amino acid sequence of SEQ ID NO: 100, and the HCDR3 comprises an amino acid sequence of SEQ ID NO: 101; and in the light chain variable region, the LCDR1 comprises an amino acid sequence of SEQ ID NO: 102, the LCDR2 comprises an amino acid sequence of SEQ ID NO: 103, and the LCDR3 comprises an amino acid sequence of SEQ ID NO: 104; or
l. in the heavy chain variable region, the HCDR1 comprises an amino acid sequence of SEQ ID NO: 147, the HCDR2 comprises an amino acid sequence of SEQ ID NO: 148, and the HCDR3 comprises an amino acid sequence of SEQ ID NO: 149; and in the light chain variable region, the LCDR1 comprises an amino acid sequence of SEQ ID NO: 150, the LCDR2 comprises an amino acid sequence of SEQ ID NO: 151, and the LCDR3 comprises an amino acid sequence of SEQ ID NO: 152; or
m. in the heavy chain variable region, the HCDR1 comprises an amino acid sequence of SEQ ID NO: 153, the HCDR2 comprises an amino acid sequence of SEQ ID NO: 154, and the HCDR3 comprises an amino acid sequence of SEQ ID NO: 155; and in the light chain variable region, the LCDR1 comprises an amino acid sequence of SEQ ID NO: 114, the LCDR2 comprises an amino acid sequence of SEQ ID NO: 133, and the LCDR3 comprises an amino acid sequence of SEQ ID NO: 119; or
n. in the heavy chain variable region, the HCDR1 comprises an amino acid sequence of SEQ ID NO: 58, the HCDR2 comprises an amino acid sequence of SEQ ID NO: 156, and the HCDR3 comprises an amino acid sequence of SEQ ID NO: 157; and in the light chain variable region, the LCDR1 comprises an amino acid sequence of SEQ ID NO: 158, the LCDR2 comprises an amino acid sequence of SEQ ID NO: 62, and the LCDR3 comprises an amino acid sequence of SEQ ID NO: 159; or
o. in the heavy chain variable region, the HCDR1 comprises an amino acid sequence of SEQ ID NO: 105, the HCDR2 comprises an amino acid sequence of SEQ ID NO: 160, and the HCDR3 comprises an amino acid sequence of SEQ ID NO: 161; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 162.

In some embodiments, provided is the anti-ILT4 antibody according to any one of the above, wherein:
a. in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119; or
b. in the heavy chain variable region, the HCDR1 comprises an amino acid sequence of SEQ ID NO: 135, the HCDR2 comprises an amino acid sequence of SEQ ID NO: 203, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140; or
c. in the heavy chain variable region, the HCDR1 comprises an amino acid sequence of SEQ ID NO: 111, the HCDR2 comprises an amino acid sequence of SEQ ID NO: 173, and the HCDR3 comprises an amino acid sequence of SEQ ID NO: 113; and in the light chain variable region, the LCDR1 comprises an amino acid sequence of SEQ ID NO: 114, the LCDR2 comprises an amino acid sequence of SEQ ID NO: 115, and the LCDR3 comprises an amino acid sequence of SEQ ID NO: 116; or
d. in the heavy chain variable region, the HCDR1 comprises an amino acid sequence of SEQ ID NO: 69, the HCDR2 comprises an amino acid sequence of SEQ ID NO: 189, and the HCDR3 comprises an amino acid sequence of SEQ ID NO: 71; and in the light chain variable region, the LCDR1 comprises an amino acid sequence of SEQ ID NO: 61, the LCDR2 comprises an amino acid sequence of SEQ ID NO: 62, and the LCDR3 comprises an amino acid sequence of SEQ ID NO: 72; or
e. in the heavy chain variable region, the HCDR1 comprises an amino acid sequence of SEQ ID NO: 165, the HCDR2 comprises an amino acid sequence of SEQ ID NO: 166, and the HCDR3 comprises an amino acid sequence of SEQ ID NO: 167; and in the light chain variable region, the LCDR1 comprises an amino acid sequence of SEQ ID NO: 224, the LCDR2 comprises an amino acid sequence of SEQ ID NO: 169, and the LCDR3 comprises an amino acid sequence of SEQ ID NO: 170; or
f. in the heavy chain variable region, the HCDR1 comprises an amino acid sequence of SEQ ID NO: 105, the HCDR2 comprises an amino acid sequence of SEQ ID NO: 125, and the HCDR3 comprises an amino acid sequence of SEQ ID NO: 126; and in the light chain variable region, the LCDR1 comprises an amino acid sequence of SEQ ID NO: 127, the LCDR2 comprises an amino acid sequence of SEQ ID NO: 109, and the LCDR3 comprises an amino acid sequence of SEQ ID NO: 128; or
g. in the heavy chain variable region, the HCDR1 comprises an amino acid sequence of SEQ ID NO: 141, the HCDR2 comprises an amino acid sequence of SEQ ID NO: 215, and the HCDR3 comprises an amino acid sequence of SEQ ID NO: 216; and in the light chain variable region, the LCDR1 comprises an amino acid sequence of SEQ ID NO: 144, the LCDR2 comprises an amino acid sequence of SEQ ID NO: 145, and the LCDR3 comprises an amino acid sequence of SEQ ID NO: 146.

In some embodiments, provided is the anti-ILT4 antibody according to any one of the above, wherein:
a. in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119; or
b. in the heavy chain variable region, the HCDR1 comprises an amino acid sequence of SEQ ID NO: 135, the HCDR2 comprises an amino acid sequence of SEQ ID NO: 203, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140.

In some embodiments, provided is the anti-ILT4 antibody according to any one of the above, wherein:
in the heavy chain variable region, the HCDR1 comprises an amino acid sequence set forth in SEQ ID NO: 105, the HCDR2 comprises an amino acid sequence set forth in SEQ ID NO: 120, and the HCDR3 comprises an amino acid sequence set forth in SEQ ID NO: 121; and in the light chain variable region, the LCDR1 comprises an amino acid sequence set forth in SEQ ID NO: 114, the LCDR2 comprises an amino acid sequence set forth in SEQ ID NO: 109, and the LCDR3 comprises an amino acid sequence set forth in SEQ ID NO: 119.

In some embodiments, provided is the anti-ILT4 antibody according to any one of the above, wherein:
p. in the heavy chain variable region, the HCDR1 comprises an amino acid sequence of SEQ ID NO: 52, the HCDR2 comprises an amino acid sequence of SEQ ID NO: 53, and the HCDR3 comprises an amino acid sequence of SEQ ID NO: 54; and in the light chain variable region, the LCDR1 comprises an amino acid sequence of SEQ ID NO: 55, the LCDR2 comprises an amino acid sequence of SEQ ID NO: 56, and the LCDR3 comprises an amino acid sequence of SEQ ID NO: 57; or
q. in the heavy chain variable region, the HCDR1 comprises an amino acid sequence of SEQ ID NO: 58, the HCDR2 comprises an amino acid sequence of SEQ ID NO: 59, and the HCDR3 comprises an amino acid sequence of SEQ ID NO: 60; and in the light chain variable region, the LCDR1 comprises an amino acid sequence of SEQ ID NO: 61, the LCDR2 comprises an amino acid sequence of SEQ ID NO: 62, and the LCDR3 comprises an amino acid sequence of SEQ ID NO: 63; or
r. in the heavy chain variable region, the HCDR1 comprises an amino acid sequence of SEQ ID NO: 64, the HCDR2 comprises an amino acid sequence of SEQ ID NO: 65, and the HCDR3 comprises an amino acid sequence of SEQ ID NO: 66; and in the light chain variable region, the LCDR1 comprises an amino acid sequence of SEQ ID NO: 67, the LCDR2 comprises an amino acid sequence of SEQ ID NO: 56, and the LCDR3 comprises an amino acid sequence of SEQ ID NO: 68; or
s. in the heavy chain variable region, the HCDR1 comprises an amino acid sequence of SEQ ID NO: 73, the HCDR2 comprises an amino acid sequence of SEQ ID NO: 74, and the HCDR3 comprises an amino acid sequence of SEQ ID NO: 75; and in the light chain variable region, the LCDR1 comprises an amino acid sequence of SEQ ID NO: 76, the LCDR2 comprises an amino acid sequence of SEQ ID NO: 62, and the LCDR3 comprises an amino acid sequence of SEQ ID NO: 77; or
t. in the heavy chain variable region, the HCDR1 comprises an amino acid sequence of SEQ ID NO: 78, the HCDR2 comprises an amino acid sequence of SEQ ID NO: 79, and the HCDR3 comprises an amino acid sequence of SEQ ID NO: 80; and in the light chain variable region, the LCDR1 comprises an amino acid sequence of SEQ ID NO: 81, the LCDR2 comprises an amino acid sequence of SEQ ID NO: 62, and the LCDR3 comprises an amino acid sequence of SEQ ID NO: 82; or
u. in the heavy chain variable region, the HCDR1 comprises an amino acid sequence of SEQ ID NO: 52, the HCDR2 comprises an amino acid sequence of SEQ ID NO: 83, and the HCDR3 comprises an amino acid sequence of SEQ ID NO: 84; and in the light chain variable region, the LCDR1 comprises an amino acid sequence of SEQ ID NO: 85, the LCDR2 comprises an amino acid sequence of SEQ ID NO: 62, and the LCDR3 comprises an amino acid sequence of SEQ ID NO: 86; or
v. in the heavy chain variable region, the HCDR1 comprises an amino acid sequence of SEQ ID NO: 95, the HCDR2 comprises an amino acid sequence of SEQ ID NO: 96, and the HCDR3 comprises an amino acid sequence of SEQ ID NO: 97; and in the light chain variable region, the LCDR1 comprises an amino acid sequence of SEQ ID NO: 85, the LCDR2 comprises an amino acid sequence of SEQ ID NO: 98, and the LCDR3 comprises an amino acid sequence of SEQ ID NO: 68; or
w. in the heavy chain variable region, the HCDR1 comprises an amino acid sequence of SEQ ID NO: 105, the HCDR2 comprises an amino acid sequence of SEQ ID NO: 106, and the HCDR3 comprises an amino acid sequence of SEQ ID NO: 107; and in the light chain variable region, the LCDR1 comprises an amino acid sequence of SEQ ID NO: 108, the LCDR2 comprises an amino acid sequence of SEQ ID NO: 109, and the LCDR3 comprises an amino acid sequence of SEQ ID NO: 110; or
x. in the heavy chain variable region, the HCDR1 comprises an amino acid sequence of SEQ ID NO: 105, the HCDR2 comprises an amino acid sequence of SEQ ID NO: 117, and the HCDR3 comprises an amino acid sequence of SEQ ID NO: 118; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119; or
y. in the heavy chain variable region, the HCDR1 comprises an amino acid sequence of SEQ ID NO: 129, the HCDR2 comprises an amino acid sequence of SEQ ID NO: 130, and the HCDR3 comprises an amino acid sequence of SEQ ID NO: 131; and in the light chain variable region, the LCDR1 comprises an amino acid sequence of SEQ ID NO: 132, the LCDR2 comprises an amino acid sequence of SEQ ID NO: 133, and the LCDR3 comprises an amino acid sequence of SEQ ID NO: 134.

In some embodiments, provided is the anti-ILT4 antibody according to any one of the above, wherein the HCDR1, the HCDR2, and the HCDR3 of the heavy chain variable region and the LCDR1, the LCDR2, and the LCDR3 of the light chain variable region are defined according to the Kabat numbering scheme.

In some embodiments, provided is the anti-ILT4 antibody according to any one of the above, wherein:
a. in the heavy chain variable region, the HCDR1 comprises an amino acid sequence of SEQ ID NO: 260, the HCDR2 comprises an amino acid sequence of SEQ ID NO: 261, and the HCDR3 comprises an amino acid sequence of SEQ ID NO: 262; and in the light chain variable region, the LCDR1 comprises an amino acid sequence of SEQ ID NO: 263, the LCDR2 comprises an amino acid sequence of SEQ ID NO: 264, and the LCDR3 comprises an amino acid sequence of SEQ ID NO: 119; or
b. in the heavy chain variable region, the HCDR1 comprises an amino acid sequence of SEQ ID NO: 267, the HCDR2 comprises an amino acid sequence of SEQ ID NO: 268, and the HCDR3 comprises an amino acid sequence of SEQ ID NO: 269; and in the light chain variable region, the LCDR1 comprises an amino acid sequence of SEQ ID NO: 270, the LCDR2 comprises an amino acid sequence of SEQ ID NO: 271, and the LCDR3 comprises an amino acid sequence of SEQ ID NO: 140;
wherein the CDRs are defined according to the IMGT numbering scheme.

In some embodiments, provided is the anti-ILT4 antibody according to any one of the above, wherein:
in the heavy chain variable region, the HCDR1 comprises an amino acid sequence set forth in SEQ ID NO: 260, the HCDR2 comprises an amino acid sequence set forth in SEQ ID NO: 261, and the HCDR3 comprises an amino acid sequence set forth in SEQ ID NO: 262; and in the light chain variable region, the LCDR1 comprises an amino acid sequence set forth in SEQ ID NO: 263, the LCDR2 comprises an amino acid sequence set forth in SEQ ID NO: 264, and the LCDR3 comprises an amino acid sequence set forth in SEQ ID NO:

119;
wherein the CDRs are defined according to the IMGT numbering scheme.

In some embodiments, provided is the anti-ILT4 antibody according to any one of the above, wherein:
a. in the heavy chain variable region, the HCDR1 comprises an amino acid sequence of SEQ ID NO: 265, the HCDR2 comprises an amino acid sequence of SEQ ID NO: 266, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119; or
b. in the heavy chain variable region, the HCDR1 comprises an amino acid sequence of SEQ ID NO: 272, the HCDR2 comprises an amino acid sequence of SEQ ID NO: 273, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140;
wherein the CDRs are defined according to the Chothia numbering scheme.

In some embodiments, provided is the anti-ILT4 antibody according to any one of the above, wherein:
in the heavy chain variable region, the HCDR1 comprises an amino acid sequence set forth in SEQ ID NO: 265, the HCDR2 comprises an amino acid sequence set forth in SEQ ID NO: 266, and the HCDR3 comprises an amino acid sequence set forth in SEQ ID NO: 121; and in the light chain variable region, the LCDR1 comprises an amino acid sequence set forth in SEQ ID NO: 114, the LCDR2 comprises an amino acid sequence set forth in SEQ ID NO: 109, and the LCDR3 comprises an amino acid sequence set forth in SEQ ID NO: 119;
wherein the CDRs are defined according to the Chothia numbering scheme.

In some embodiments, provided is the anti-ILT4 antibody according to any one of the above, wherein the anti-ILT4 antibody is a murine antibody, a chimeric antibody, a humanized antibody, or a fully human-derived antibody. In some embodiments, the antibody is a chimeric antibody. In some embodiments, the antibody is a fully human-derived antibody. In some embodiments, the antibody is a humanized antibody.

In some embodiments, provided is the anti-ILT4 antibody according to any one of the above, wherein the anti-ILT4 antibody comprises a framework region (FR) of a human antibody.

In some embodiments, provided is the anti-ILT4 antibody according to any one of the above, wherein:
a. the heavy chain variable region comprises an amino acid sequence having at least 70% (e.g., at least 70%, 80%, 85%, 90%, 95%, 98%, or 99%) sequence identity to SEQ ID NO: 184 or 185, and the light chain variable region comprises an amino acid sequence having at least 70% (e.g., at least 70%, 80%, 85%, 90%, 95%, 98%, or 99%) sequence identity to SEQ ID NO: 187 or 186; or the heavy chain variable region comprises an amino acid sequence having at least 70% (e.g., at least 70%, 80%, 85%, 90%, 95%, 98%, or 99%) sequence identity to SEQ ID NO: 32, and the light chain variable region comprises an amino acid sequence having at least 70% (e.g., at least 70%, 80%, 85%, 90%, 95%, 98%, or 99%) sequence identity to SEQ ID NO: 33; or
b. the heavy chain variable region comprises an amino acid sequence having at least 70% (e.g., at least 70%, 80%, 85%, 90%, 95%, 98%, or 99%) sequence identity to SEQ ID NO: 211, 205, 206, 207, 208, 209, or 210, and the light chain variable region comprises an amino acid sequence having at least 70% (e.g., at least 70%, 80%, 85%, 90%, 95%, 98%, or 99%) sequence identity to SEQ ID NO: 212, 213, or 214; or the heavy chain variable region comprises an amino acid sequence having at least 70% (e.g., at least 70%, 80%, 85%, 90%, 95%, 98%, or 99%) sequence identity to SEQ ID NO: 40, and the light chain variable region comprises an amino acid sequence having at least 70% (e.g., at least 70%, 80%, 85%, 90%, 95%, 98%, or 99%) sequence identity to SEQ ID NO: 41; or
c. the heavy chain variable region comprises an amino acid sequence having at least 70% (e.g., at least 70%, 80%, 85%, 90%, 95%, 98%, or 99%) sequence identity to SEQ ID NO: 181, 176, 177, 178, 179, or 180, and the light chain variable region comprises an amino acid sequence having at least 70% (e.g., at least 70%, 80%, 85%, 90%, 95%, 98%, or 99%) sequence identity to SEQ ID NO: 183 or 182; or the heavy chain variable region comprises an amino acid sequence having at least 70% (e.g., at least 70%, 80%, 85%, 90%, 95%, 98%, or 99%) sequence identity to SEQ ID NO: 28, and the light chain variable region comprises an amino acid sequence having at least 70% (e.g., at least 70%, 80%, 85%, 90%, 95%, 98%, or 99%) sequence identity to SEQ ID NO: 29; or
d. the heavy chain variable region comprises an amino acid sequence having at least 70% (e.g., at least 70%, 80%, 85%, 90%, 95%, 98%, or 99%) sequence identity to SEQ ID NO: 193, 190, 191, or 192, and the light chain variable region comprises an amino acid sequence having at least 70% (e.g., at least 70%, 80%, 85%, 90%, 95%, 98%, or 99%) sequence identity to SEQ ID NO: 194, 195, or 196; or the heavy chain variable region comprises an amino acid sequence having at least 70% (e.g., at least 70%, 80%, 85%, 90%, 95%, 98%, or 99%) sequence identity to SEQ ID NO: 10, and the light chain variable region comprises an amino acid sequence having at least 70% (e.g., at least 70%, 80%, 85%, 90%, 95%, 98%, or 99%) sequence identity to SEQ ID NO: 11; or
e. the heavy chain variable region comprises an amino acid sequence having at least 70% (e.g., at least 70%, 80%, 85%, 90%, 95%, 98%, or 99%) sequence identity to SEQ ID NO: 163, and the light chain variable region comprises an amino acid sequence having at least 70% (e.g., at least 70%, 80%, 85%, 90%, 95%, 98%, or 99%) sequence identity to SEQ ID NO: 225 or 164;
f. the heavy chain variable region comprises an amino acid sequence having at least 70% (e.g., at least 70%, 80%, 85%, 90%, 95%, 98%, or 99%) sequence identity to SEQ ID NO: 197 or 198, and the light chain variable region comprises an amino acid sequence having at least 70% (e.g., at least 70%, 80%, 85%, 90%, 95%, 98%, or 99%) sequence identity to SEQ ID NO: 199 or 200; or the heavy chain variable region comprises an amino acid sequence having at least 70% (e.g., at least 70%, 80%, 85%, 90%, 95%, 98%, or 99%) sequence identity to SEQ ID NO: 36, and the light chain variable region comprises an amino acid sequence having at least 70% (e.g., at least 70%, 80%, 85%, 90%, 95%, 98%, or 99%) sequence identity to SEQ ID NO: 37; or
g. the heavy chain variable region comprises an amino acid sequence having at least 70% (e.g., at least 70%, 80%, 85%, 90%, 95%, 98%, or 99%) sequence identity to SEQ ID NO: 217, 218, 219, 220, or 221, and the light chain variable region comprises an amino acid sequence having at least 70% (e.g., at least 70%, 80%, 85%, 90%, 95%, 98%, or 99%) sequence identity to SEQ ID NO: 222 or 223; or the heavy chain variable region comprises an amino acid sequence having at least 70% (e.g., at least 70%, 80%, 85%, 90%, 95%, 98%, or 99%) sequence identity to SEQ ID NO: 42, and the light chain variable region comprises an amino acid sequence having at least 70% (e.g., at least 70%, 80%, 85%, 90%, 95%, 98%, or 99%) sequence identity to SEQ ID NO: 43.

In some embodiments, provided is the anti-ILT4 antibody according to any one of the above, wherein:
a. the heavy chain variable region comprises an amino acid sequence of SEQ ID NO: 184 or 185, and the light chain variable region comprises an amino acid sequence of SEQ ID NO: 187 or 186; or the heavy chain variable region comprises an amino acid sequence of SEQ ID NO: 32, and the light chain variable region comprises an amino acid sequence of SEQ ID NO: 33; or
b. the heavy chain variable region comprises an amino acid sequence of SEQ ID NO: 211, 205, 206, 207, 208, 209, or 210, and the light chain variable region comprises an amino acid sequence of SEQ ID NO: 212, 213, or 214; or the heavy chain variable region comprises an amino acid sequence of SEQ ID NO: 40, and the light chain variable region comprises an amino acid sequence of SEQ ID NO: 41; or
c. the heavy chain variable region comprises an amino acid sequence of SEQ ID NO: 181, 176, 177, 178, 179, or 180, and the light chain variable region comprises an amino acid sequence of SEQ ID NO: 183 or 182; or the heavy chain variable region comprises an amino acid sequence of SEQ ID NO: 28, and the light chain variable region comprises an amino acid sequence of SEQ ID NO: 29; or
d. the heavy chain variable region comprises an amino acid sequence of SEQ ID NO: 193, 190, 191, or 192, and the light chain variable region comprises an amino acid sequence of SEQ ID NO: 194, 195, or 196; or the heavy chain variable region comprises an amino acid sequence of SEQ ID NO: 10, and the light chain variable region comprises an amino acid sequence of SEQ ID NO: 11; or
e. the heavy chain variable region comprises an amino acid sequence of SEQ ID NO: 163, and the light chain variable region comprises an amino acid sequence of SEQ ID NO: 225 or 164;
f. the heavy chain variable region comprises an amino acid sequence of SEQ ID NO: 197 or 198, and the light chain variable region comprises an amino acid sequence of SEQ ID NO: 199 or 200; or the heavy chain variable region comprises an amino acid sequence of SEQ ID NO: 36, and the light chain variable region comprises an amino acid sequence of SEQ ID NO: 37; or
g. the heavy chain variable region comprises an amino acid sequence of SEQ ID NO: 217, 218, 219, 220, or 221, and the light chain variable region comprises an amino acid sequence of SEQ ID NO: 222 or 223; or the heavy chain variable region comprises an amino acid sequence of SEQ ID NO: 42, and the light chain variable region comprises an amino acid sequence of SEQ ID NO: 43.

In some embodiments, provided is the anti-ILT4 antibody according to any one of the above, wherein:
a. the heavy chain variable region comprises an amino acid sequence of SEQ ID NO: 184 or 185, and the light chain variable region comprises an amino acid sequence of SEQ ID NO: 187 or 186; or
b. the heavy chain variable region comprises an amino acid sequence of SEQ ID NO: 211, 205, 206, 207, 208, 209, or 210, and the light chain variable region comprises an amino acid sequence of SEQ ID NO: 212, 213, or 214; or
c. the heavy chain variable region comprises an amino acid sequence of SEQ ID NO: 181, 176, 177, 178, 179, or 180, and the light chain variable region comprises an amino acid sequence of SEQ ID NO: 183 or 182; or
d. the heavy chain variable region comprises an amino acid sequence of SEQ ID NO: 193, 190, 191, or 192, and the light chain variable region comprises an amino acid sequence of SEQ ID NO: 194, 195, or 196; or
e. the heavy chain variable region comprises an amino acid sequence of SEQ ID NO: 163, and the light chain variable region comprises an amino acid sequence of SEQ ID NO: 225 or 164;
f. the heavy chain variable region comprises an amino acid sequence of SEQ ID NO: 197 or 198, and the light chain variable region comprises an amino acid sequence of SEQ ID NO: 199 or 200; or
g. the heavy chain variable region comprises an amino acid sequence of SEQ ID NO: 217, 218, 219, 220, or 221, and the light chain variable region comprises an amino acid sequence of SEQ ID NO: 222 or 223.

In some embodiments, provided is the anti-ILT4 antibody according to any one of the above, wherein:
a. the heavy chain variable region comprises an amino acid sequence of SEQ ID NO: 184, and the light chain variable region comprises an amino acid sequence of SEQ ID NO: 187; or
b. the heavy chain variable region comprises an amino acid sequence of SEQ ID NO: 211, and the light chain variable region comprises an amino acid sequence of SEQ ID NO: 212; or
c. the heavy chain variable region comprises an amino acid sequence of SEQ ID NO: 181, and the light chain variable region comprises an amino acid sequence of SEQ ID NO: 183; or
d. the heavy chain variable region comprises an amino acid sequence of SEQ ID NO: 193, and the light chain variable region comprises an amino acid sequence of SEQ ID NO: 194; or
e. the heavy chain variable region comprises an amino acid sequence of SEQ ID NO: 163, and the light chain variable region comprises an amino acid sequence of SEQ ID NO: 225;
f. the heavy chain variable region comprises an amino acid sequence of SEQ ID NO: 197, and the light chain variable region comprises an amino acid sequence of SEQ ID NO: 199; or
g. the heavy chain variable region comprises an amino acid sequence of SEQ ID NO: 217, and the light chain variable region comprises an amino acid sequence of SEQ ID NO: 222.

In some embodiments, provided is the anti-ILT4 antibody according to any one of the above, wherein:

a. the heavy chain variable region comprises an amino acid sequence of SEQ ID NO: 184, and the light chain variable region comprises an amino acid sequence of SEQ ID NO: 187; or
b. the heavy chain variable region comprises an amino acid sequence of SEQ ID NO: 211, and the light chain variable region comprises an amino acid sequence of SEQ ID NO: 212.

In some embodiments, provided is the anti-ILT4 antibody according to any one of the above, wherein:

the heavy chain variable region comprises an amino acid sequence set forth in SEQ ID NO: 184, and the light chain variable region comprises an amino acid sequence set forth in SEQ ID NO: 187.

In some embodiments, provided is the anti-ILT4 antibody according to any one of the above, wherein:
a. the heavy chain variable region comprises an amino acid sequence of SEQ ID NO: 32, and the light chain variable region comprises an amino acid sequence of SEQ ID NO: 33; or
b. the heavy chain variable region comprises an amino acid sequence of SEQ ID NO: 40, and the light chain variable region comprises an amino acid sequence of SEQ ID NO: 41; or
c. the heavy chain variable region comprises an amino acid sequence of SEQ ID NO: 28, and the light chain variable region comprises an amino acid sequence of SEQ ID NO: 29; or
d. the heavy chain variable region comprises an amino acid sequence of SEQ ID NO: 10, and the light chain variable region comprises an amino acid sequence of SEQ ID NO: 11; or
f. the heavy chain variable region comprises an amino acid sequence of SEQ ID NO: 36, and the light chain variable region comprises an amino acid sequence of SEQ ID NO: 37; or
g. the heavy chain variable region comprises an amino acid sequence of SEQ ID NO: 42, and the light chain variable region comprises an amino acid sequence of SEQ ID NO: 43.

In some embodiments, provided is the anti-ILT4 antibody according to any one of the above, wherein the heavy chain variable region comprises a FR1, a FR2, and a FR3 that are derived from IGHV4-31*01 and a FR4 derived from IGHJ1*01, and the FRs are unsubstituted or have one or more amino acid substitutions selected from the group consisting of 1E, 27S, 30T, 44K, 71R, and 76S; and/or the light chain variable region comprises a FR1, a FR2, and a FR3 that are derived from IGKV1-39*01 and a FR4 derived from IGKJ4*01, and the FRs are unsubstituted or have one or more amino acid substitutions selected from the group consisting of 43S, 48V, and 71Y. In some embodiments, provided is the anti-ILT4 antibody, wherein in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121, and the FRs of the heavy chain variable region comprise one or more amino acid substitutions selected from the group consisting of 1E, 27S, 30T, 44K, 71R, and 76S; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119, and the FRs of the light chain variable region comprise one or more amino acid substitutions selected from the group consisting of 43S, 48V, and 71Y. In some embodiments, the variable regions and CDRs described above are defined according to the Kabat numbering scheme.

In some embodiments, provided is the anti-ILT4 antibody according to any one of the above, wherein the heavy chain variable region comprises a FR1, a FR2, and a FR3 that are derived from IGHV1-18*01 and a FR4 derived from IGHJ6*01, and the FRs are unsubstituted or have one or more amino acid substitutions selected from the group consisting of 1E, 43E, 69L, 71V, 73K, 76N, and 93T; and/or the light chain variable region comprises a FR1, a FR2, and a FR3 that are derived from IGKV4-1*01 or IGKV3-11*01 and a FR4 derived from IGKJ4*01, and the FRs are unsubstituted or have one or more amino acid substitutions selected from the group consisting of 4L, 43P, 45K, 581, 68R, and 85T. In some embodiments, provided is the anti-ILT4 antibody, wherein in the heavy chain variable region, the HCDR1 comprises an amino acid sequence of SEQ ID NO: 135, the HCDR2 comprises an amino acid sequence of SEQ ID NO: 203, 136, 201, 202, or 204, and the HCDR3 comprises an amino acid sequence of SEQ ID NO: 137, and the FRs of the heavy chain variable region comprise one or more amino acid substitutions selected from the group consisting of 1E, 43E, 69L, 71V, 73K, 76N, and 93T; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140, and the FRs of the light chain variable region comprise one or more amino acid substitutions selected from the group consisting of 4L, 43P, 45K, 581, 68R, and 85T. In some embodiments, the variable regions and CDRs described above are defined according to the Kabat numbering scheme.

In some embodiments, provided is the anti-ILT4 antibody according to any one of the above, wherein the heavy chain variable region comprises a FR1, a FR2, and a FR3 that are derived from IGHV1-3*01 and a FR4 derived from IGHJ6*01, and the FRs are unsubstituted or have one or more amino acid substitutions selected from the group consisting of 1E, 38K, 44S, 69L, 71V, 73K, and 75G; and/or the light chain variable region comprises a FR1, a FR2, and a FR3 that are derived from IGKV1-39*01 and a FR4 derived from IGKJ2*01, and the FRs are unsubstituted or have one or more amino acid substitutions selected from the group consisting of 43S, 48V, and 71Y. In some embodiments, provided is the anti-ILT4 antibody, wherein in the heavy chain variable region, the HCDR1 comprises an amino acid sequence of SEQ ID NO: 111, the HCDR2 comprises an amino acid sequence of SEQ ID NO: 173, 112, 174, or 175, and the HCDR3 comprises an amino acid sequence of SEQ ID NO: 113, and the FRs of the heavy chain variable region comprise one or more amino acid substitutions selected from the group consisting of 1E, 38K, 44S, 69L, 71V, 73K, and 75G; and in the light chain variable region, the LCDR1 comprises an amino acid sequence of SEQ ID NO: 114, the LCDR2 comprises an amino acid sequence of SEQ ID NO: 115, and the LCDR3 comprises an amino acid sequence of SEQ ID NO: 116, and the FRs of the light chain variable region comprise one or more amino acid substitutions selected from the group consisting of 43S, 48V, and 71Y.

In some embodiments, the variable regions and CDRs described above are defined according to the Kabat numbering scheme.

In some embodiments, provided is the anti-ILT4 antibody according to any one of the above, wherein the heavy chain variable region comprises a FR1, a FR2, and a FR3 that are derived from IGHV3-21*01 and a FR4 derived from IGHJ1*01, and the FRs are unsubstituted or have a 49A substitution; and/or the light chain variable region comprises a FR1, a FR2, and a FR3 that are derived from IGKV1-33*01 and a FR4 derived from IGKJ4*01, and the FRs are unsubstituted or have one or more amino acid substitutions selected from the group consisting of 39R, 42Q, 43S, and 85V. In some embodiments, provided is the anti-ILT4 antibody, wherein in the heavy chain variable region, the HCDR1 comprises an amino acid sequence of SEQ ID NO: 69, the HCDR2 comprises an amino acid sequence of SEQ ID NO: 189, 70, or 188, and the HCDR3 comprises an amino acid sequence of SEQ ID NO: 71, and the FRs of the heavy chain variable region comprise a 49A substitution; and in the light chain variable region, the LCDR1 comprises an amino acid sequence of SEQ ID NO: 61, the LCDR2 comprises an amino acid sequence of SEQ ID NO: 62, and the LCDR3 comprises an amino acid sequence of SEQ ID NO: 72, and the FRs of the light chain variable region comprise one or more amino acid substitutions selected from the group consisting of 39R, 42Q, 43S, and 85V. In some embodiments, the variable regions and CDRs described above are defined according to the Kabat numbering scheme.

In some embodiments, provided is the anti-ILT4 antibody according to any one of the above, wherein the heavy chain variable region comprises a FR1, a FR2, and a FR3 that are derived from IGHV4-31*02 and a FR4 derived from IGHJ6*01, and the FRs are unsubstituted or have one or more amino acid substitutions selected from the group consisting of 1E, 27Y, 30T, 42E, 44K, and 71R; and/or the light chain variable region comprises a FR1, a FR2, and a FR3 that are derived from IGKV1-39*01 and a FR4 derived from IGKJ4*01, and the FRs are unsubstituted or have one or more amino acid substitutions selected from the group consisting of 43S, 48L, and 71Y. In some embodiments, provided is the anti-ILT4 antibody, wherein in the heavy chain variable region, the HCDR1 comprises an amino acid sequence of SEQ ID NO: 105, the HCDR2 comprises an amino acid sequence of SEQ ID NO: 125, and the HCDR3 comprises an amino acid sequence of SEQ ID NO: 126, and the FRs of the heavy chain variable region comprise one or more amino acid substitutions selected from the group consisting of 1E, 27Y, 30T, 42E, 44K, and 71R; and in the light chain variable region, the LCDR1 comprises an amino acid sequence of SEQ ID NO: 127, the LCDR2 comprises an amino acid sequence of SEQ ID NO: 109, and the LCDR3 comprises an amino acid sequence of SEQ ID NO: 128, and the FRs of the light chain variable region comprise one or more amino acid substitutions selected from the group consisting of 43S, 48L, and 71Y. In some embodiments, the variable regions and CDRs described above are defined according to the Kabat numbering scheme.

In some embodiments, provided is the anti-ILT4 antibody according to any one of the above, wherein the heavy chain variable region comprises a FR1, a FR2, and a FR3 that are derived from IGHV1-18*01 and a FR4 derived from IGHJ6*01, and the FRs are unsubstituted or have one or more amino acid substitutions selected from the group consisting of 1E, 21, 44A, 46K, and 76T; and/or the light chain variable region comprises a FR1, a FR2, and a FR3 that are derived from IGKV4-1*01 or IGKV2-28*01 and a FR4 derived from IGKJ4*01, and the FRs are unsubstituted or have one or more amino acid substitutions selected from the group consisting of 43S and 48L. In some embodiments, provided is the anti-ILT4 antibody, wherein in the heavy chain variable region, the HCDR1 comprises an amino acid sequence of SEQ ID NO: 141, the HCDR2 comprises an amino acid sequence of SEQ ID NO: 215 or 142, and the HCDR3 comprises an amino acid sequence of SEQ ID NO: 216 or 143, and the FRs of the heavy chain variable region comprise one or more amino acid substitutions selected from the group consisting of 1E, 21, 44A, 46K, and 76T; and in the light chain variable region, the LCDR1 comprises an amino acid sequence of SEQ ID NO: 144, the LCDR2 comprises an amino acid sequence of SEQ ID NO: 145, and the LCDR3 comprises an amino acid sequence of SEQ ID NO: 146, and the FRs of the light chain variable region comprise one or more amino acid substitutions selected from the group consisting of 43S and 48L. In some embodiments, the variable regions and CDRs described above are defined according to the Kabat numbering scheme.

In some embodiments, provided is the anti-ILT4 antibody according to any one of the above, wherein the anti-ILT4 antibody is an antibody fragment; in some embodiments, the antibody fragment is selected from the group consisting of Fab, Fab′, F(ab′)2, Fd, Fv, scFv, dsFv, and dAb.

In some embodiments, provided is the anti-ILT4 antibody according to any one of the above, wherein the anti-ILT4 antibody comprises a heavy chain constant region and a light chain constant region. In some embodiments, the heavy chain constant region is a human IgG1 or IgG4 heavy chain constant region. In some embodiments, the heavy chain constant region comprises 234A and 235A mutations; the mutations are numbered according to the EU index. In some embodiments, the light chain constant region is a human k light chain constant region. In some embodiments, the heavy chain constant region comprises an amino acid sequence of SEQ ID NO: 240 or 171, and the light chain constant region comprises an amino acid sequence of SEQ ID NO: 172. In some embodiments, the heavy chain constant region comprises an amino acid sequence of SEQ ID NO: 240, and the light chain constant region comprises an amino acid sequence of SEQ ID NO: 172.

In some embodiments, provided is the anti-ILT4 antibody according to any one of the above, wherein the anti-ILT4 antibody comprises a heavy chain and a light chain, wherein:
a. the heavy chain comprises an amino acid sequence having at least 70% (e.g., at least 70%, 80%, 85%, 90%, 95%, 98%, or 99%) sequence identity to SEQ ID NO: 228, and the light chain comprises an amino acid sequence having at least 70% (e.g., at least 70%, 80%, 85%, 90%, 95%, 98%, or 99%) sequence identity to SEQ ID NO: 229; or
b. the heavy chain comprises an amino acid sequence having at least 70% (e.g., at least 70%, 80%, 85%, 90%, 95%, 98%, or 99%) sequence identity to SEQ ID NO: 234, and the light chain comprises an amino acid sequence having at least 70% (e.g., at least 70%, 80%, 85%, 90%, 95%, 98%, or 99%) sequence identity to SEQ ID NO: 235; or
c. the heavy chain comprises an amino acid sequence having at least 70% (e.g., at least 70%, 80%, 85%, 90%, 95%, 98%, or 99%) sequence identity to SEQ ID NO: 226, and the light chain comprises an amino acid sequence having at least 70% (e.g., at least 70%, 80%, 85%, 90%, 95%, 98%, or 99%) sequence identity to SEQ ID NO: 227; or
d. the heavy chain comprises an amino acid sequence having at least 70% (e.g., at least 70%, 80%, 85%, 90%, 95%, 98%, or 99%) sequence identity to SEQ ID NO: 230, and the light chain comprises an amino acid sequence having at least 70% (e.g., at least 70%, 80%, 85%, 90%, 95%, 98%, or 99%) sequence identity to SEQ ID NO: 231; or
e. the heavy chain comprises an amino acid sequence having at least 70% (e.g., at least 70%, 80%, 85%, 90%, 95%, 98%, or 99%) sequence identity to SEQ ID NO: 238, and the light chain comprises an amino acid sequence having at least 70% (e.g., at least 70%, 80%, 85%, 90%, 95%, 98%, or 99%) sequence identity to SEQ ID NO: 239; or
f. the heavy chain comprises an amino acid sequence having at least 70% (e.g., at least 70%, 80%, 85%, 90%, 95%, 98%, or 99%) sequence identity to SEQ ID NO: 232, and the light chain comprises an amino acid sequence having at least 70% (e.g., at least 70%, 80%, 85%, 90%, 95%, 98%, or 99%) sequence identity to SEQ ID NO: 233; or
g. the heavy chain comprises an amino acid sequence having at least 70% (e.g., at least 70%, 80%, 85%, 90%, 95%, 98%, or 99%) sequence identity to SEQ ID NO: 236, and the light chain comprises an amino acid sequence having at least 70% (e.g., at least 70%, 80%, 85%, 90%, 95%, 98%, or 99%) sequence identity to SEQ ID NO: 237.

In some embodiments, provided is the anti-ILT4 antibody according to any one of the above, wherein the anti-ILT4 antibody comprises a heavy chain and a light chain, wherein:
a. the heavy chain comprises an amino acid sequence of SEQ ID NO: 228, and the light chain comprises an amino acid sequence of SEQ ID NO: 229;
b. the heavy chain comprises an amino acid sequence of SEQ ID NO: 234, and the light chain comprises an amino acid sequence of SEQ ID NO: 235;
c. the heavy chain comprises an amino acid sequence of SEQ ID NO: 226, and the light chain comprises an amino acid sequence of SEQ ID NO: 227;
d. the heavy chain comprises an amino acid sequence of SEQ ID NO: 230, and the light chain comprises an amino acid sequence of SEQ ID NO: 231;
e. the heavy chain comprises an amino acid sequence of SEQ ID NO: 238, and the light chain comprises an amino acid sequence of SEQ ID NO: 239;
f. the heavy chain comprises an amino acid sequence of SEQ ID NO: 232, and the light chain comprises an amino acid sequence of SEQ ID NO: 233;
g. the heavy chain comprises an amino acid sequence of SEQ ID NO: 236, and the light chain comprises an amino acid sequence of SEQ ID NO: 237.

In some embodiments, provided is the anti-ILT4 antibody according to any one of the above, wherein the anti-ILT4 antibody comprises a heavy chain and a light chain, wherein:
a. the heavy chain comprises an amino acid sequence of SEQ ID NO: 228, and the light chain comprises an amino acid sequence of SEQ ID NO: 229;
b. the heavy chain comprises an amino acid sequence of SEQ ID NO: 234, and the light chain comprises an amino acid sequence of SEQ ID NO: 235.

In some embodiments, provided is the anti-ILT4 antibody according to any one of the above, wherein the anti-ILT4 antibody comprises a heavy chain and a light chain, wherein: the heavy chain comprises an amino acid sequence set forth in SEQ ID NO: 228, and the light chain comprises an amino acid sequence set forth in SEQ ID NO: 229.

In some embodiments, the present disclosure further provides an isolated anti-ILT4 antibody that competes for binding to human ILT4 or an epitope thereof with the anti-ILT4 antibody according to any one of the foregoing.

In some embodiments, the anti-ILT4 antibody provided by the present disclosure specifically binds to human ILT4 and does not bind to other LILR family members: ILT2, ILT3, LILRB5, LILRA1, LILRA2, LILRA3, LILRA4, LILRA5, and/or LILRA6.

In some embodiments, the ability of the isolated anti-ILT4 antibody of the present disclosure to bind to ILT4 protein is higher than that of a control antibody. Specifically, the isolated anti-ILT4 antibody of the present disclosure binds to human ILT4 with a KD value of less than 1×10⁻⁸ M (e.g., less than 8×10⁻⁹ M, less than 5×10⁻⁹ M, less than 2×10⁻⁹ M, less than 1×10⁻⁹ M, less than 8×10⁻¹⁰ M, less than 5×10⁻¹⁰ M, less than 2×10⁻¹⁰ M, less than 1×10⁻¹⁰ M, less than 8×10⁻¹¹ M, or less than 5×10⁻¹¹ M), as measured by Biacore.

In some embodiments, a humanized anti-ILT4 antibody provided by the present disclosure is capable of effectively removing the inhibitory effect of ILT4 and promoting the secretion of higher levels of mIL-2 by 3A9 cells.

In some embodiments, a humanized anti-ILT4 antibody provided by the present disclosure is capable of effectively promoting the transformation of macrophages to the M1 phenotype and the secretion of high levels of the proinflammatory cytokine TNF-α.

In some embodiments, a humanized anti-ILT4 antibody provided by the present disclosure (e.g., huP2-G9) is capable of inducing dendritic cells to secrete higher levels of proinflammatory cytokines or chemokines in an immunosuppressive environment with IL-10.

In some embodiments, a humanized anti-ILT4 antibody provided by the present disclosure is capable of effectively blocking the binding of ILT4 to HLA-G.

In some embodiments, a humanized anti-ILT4 antibody provided by the present disclosure is capable of effectively blocking the binding of ILT4 to HLA-G, and the blocking activity of the humanized anti-ILT4 antibody is stronger than that of the control antibodies MK and JTX.

In another aspect, the present disclosure provides a pharmaceutical composition comprising the anti-ILT4 antibody according to any one of the above and one or more pharmaceutically acceptable carriers, diluents, or excipients. In some embodiments, the pharmaceutical composition further comprises at least one second therapeutic agent. In some embodiments, the second therapeutic agent is an anti-PD-1 antibody.

In another aspect, the present disclosure provides an isolated nucleic acid encoding the anti-ILT4 antibody according to any one of the above.

In another aspect, the present disclosure provides a vector comprising the aforementioned nucleic acid molecule.

In another aspect, the present disclosure provides a host cell comprising the isolated nucleic acid according to any one of the above.

In another aspect, the present disclosure provides a method for blocking the binding of ILT4 to HLA-G in a human subject in need thereof, the method comprising administering to the human subject an effective amount of the anti-ILT4 antibody according to any one of the above.

In another aspect, the present disclosure provides a method for detecting an ILT4 peptide or a fragment thereof in a sample, the method comprising contacting the sample with the anti-ILT4 antibody according to any one of the above and detecting the presence of a complex between the anti-ILT4 antibody and the ILT4 peptide or the fragment thereof, wherein that the complex is detected indicates the presence of the ILT4 peptide or the fragment thereof.

In another aspect, the present disclosure provides a method for treating a disease, the method comprising a step of administering to a subject the anti-ILT4 antibody or the pharmaceutical composition according to any one of the above.

In another aspect, the present disclosure provides use of the anti-ILT4 antibody or the pharmaceutical composition according to any one of the above in the preparation of a medicament for treating a disease.

In another aspect, the present disclosure provides the anti-ILT4 antibody or the pharmaceutical composition according to any one of the above for use as a medicament. In some embodiments, the medicament is used for treating a disease.

In some embodiments, the disease according to any one of the above is a tumor. In some embodiments, the disease is selected from the group consisting of astrocytoma (e.g., anaplastic astrocytoma), glioblastoma, bladder cancer, bone cancer, brain cancer, breast cancer (e.g., breast cancer characterized by BRCA1 and/or BRCA2 mutations), carcinoid, cervical cancer, choroid plexus papilloma, colorectal cancer (e.g., colon cancer and rectal cancer), fallopian tube cancer, ependymoma, gallbladder cancer, gastric cancer, head and neck cancer, idiopathic myelofibrosis, renal cancer or renal cell carcinoma (e.g., rhabdoid tumor of the kidney and Wilms' tumor), renal pelvis tumor, leukemia, liver cancer (e.g., hepatocellular carcinoma), esophageal cancer (also known as “oesophageal cancer”, e.g., esophageal squamous cell carcinoma), lung cancer (e.g., non-small cell lung cancer and small cell lung cancer), lymphoma, medulloblastoma, melanoma, meningioma, Merkel cell carcinoma, mesothelioma, multiple myeloma, neuroblastoma, oligodendroglioma, ovarian cancer, peritoneal tumor, pancreatic cancer, polycythemia vera, primary neuroectodermal tumor, prostate cancer, retinoblastoma, salivary gland cancer, sarcoma (e.g., chondrosarcoma, Ewing sarcoma, osteosarcoma, rhabdomyosarcoma, synovial sarcoma, and soft tissue sarcoma), small intestine cancer, colorectal cancer, squamous cell carcinoma (e.g., cutaneous squamous cell carcinoma), thyroid cancer, endometrial cancer, vestibular schwannoma, blastoma, teratoma, pituitary cancer, vulva cancer, thymoma, testicular cancer, bile duct cancer, pheochromocytoma, paraganglioma, and adenoid cystic carcinoma. In some embodiments, the disease is selected from the group consisting of ovarian cancer, lung cancer, esophageal squamous cell carcinoma, colorectal cancer, renal cell carcinoma, and melanoma.

In some embodiments, provided is the aforementioned method for treating a disease, wherein the method further comprises using a second therapeutic agent. In some embodiments, the second therapeutic agent comprises an immune checkpoint inhibitor. In some embodiments, the second therapeutic agent is an anti-PD-1 antibody.

In some embodiments, provided is the aforementioned method for treating a disease, wherein the anti-PD-1 antibody comprises a heavy chain variable region PD-1-VH and a light chain variable region PD-1-VL, wherein:

the PD-1-VH comprises: a PD-1-HCDR1 comprising an amino acid sequence of SEQ ID NO: 250, a PD-1-HCDR2 comprising an amino acid sequence of SEQ ID NO: 251, and a PD-1-HCDR3 comprising an amino acid sequence of SEQ ID NO: 252, and the PD-1-VL comprises: a PD-1-LCDR1 comprising an amino acid sequence of SEQ ID NO: 253, a PD-1-LCDR2 comprising an amino acid sequence of SEQ ID NO: 254, and a PD-1-LCDR3 comprising an amino acid sequence of SEQ ID NO: 255.

In some embodiments, provided is the aforementioned method for treating a disease, wherein the anti-PD-1 antibody comprises a heavy chain variable region PD-1-VH and a light chain variable region PD-1-VL, wherein:

the PD-1-VH comprises an amino acid sequence of SEQ ID NO: 256, and the PD-1-VL comprises an amino acid sequence of SEQ ID NO: 257.

In some embodiments, provided is the aforementioned method for treating a disease, wherein the anti-PD-1 antibody comprises a heavy chain and a light chain, wherein: the heavy chain comprises an amino acid sequence of SEQ ID NO: 248, and the light chain comprises an amino acid sequence of SEQ ID NO: 249.

In another aspect, provided is use of a combination of the anti-ILT4 antibody and the anti-PD-1 antibody according to any one of the above in the preparation of a medicament for treating a disease.

In some embodiments, the aforementioned medicament is used for treating a disease; preferably, the disease is a tumor; more preferably, the disease is a disease selected from the group consisting of astrocytoma (e.g., anaplastic astrocytoma), glioblastoma, bladder cancer, bone cancer, brain cancer, breast cancer (e.g., breast cancer characterized by BRCA1 and/or BRCA2 mutations), carcinoid, cervical cancer, choroid plexus papilloma, colorectal cancer (e.g., colon cancer and rectal cancer), fallopian tube cancer, ependymoma, gallbladder cancer, gastric cancer, head and neck cancer, idiopathic myelofibrosis, renal cancer or renal cell carcinoma (e.g., rhabdoid tumor of the kidney and Wilms' tumor), renal pelvis tumor, leukemia, liver cancer (e.g., hepatocellular carcinoma), esophageal cancer (also known as “oesophageal cancer”, e.g., esophageal squamous cell carcinoma), lung cancer (e.g., non-small cell lung cancer and small cell lung cancer), lymphoma, medulloblastoma, melanoma, meningioma, Merkel cell carcinoma, mesothelioma, multiple myeloma, neuroblastoma, oligodendroglioma, ovarian cancer, peritoneal tumor, pancreatic cancer, polycythemia vera, primary neuroectodermal tumor, prostate cancer, retinoblastoma, salivary gland cancer, sarcoma (e.g., chondrosarcoma, Ewing sarcoma, osteosarcoma, rhabdomyosarcoma, synovial sarcoma, and soft tissue sarcoma), small intestine cancer, colorectal cancer, squamous cell carcinoma (e.g., cutaneous squamous cell carcinoma), thyroid cancer, endometrial cancer, vestibular schwannoma, blastoma, teratoma, pituitary cancer, vulva cancer, thymoma, testicular cancer, bile duct cancer, pheochromocytoma, paraganglioma, and adenoid cystic carcinoma. In some embodiments, the aforementioned medicament is used for treating a disease selected from the group consisting of ovarian cancer, lung cancer, esophageal squamous cell carcinoma, colorectal cancer, renal cell carcinoma, and melanoma.

In some embodiments, provided is the aforementioned use of a combination of the anti-ILT4 antibody and the anti-PD-1 antibody in the preparation of a medicament for treating a disease, wherein the anti-ILT4 antibody and the anti-PD-1 antibody are in the same container or different containers.

In some embodiments, provided is the aforementioned use of a combination of the anti-ILT4 antibody and the anti-PD-1 antibody in the preparation of a medicament for treating a disease, wherein the anti-PD-1 antibody comprises a heavy chain variable region PD-1-VH and a light chain variable region PD-1-VL, wherein:

the PD-1-VH comprises: a PD-1-HCDR1 comprising an amino acid sequence of SEQ ID NO: 250, a PD-1-HCDR2 comprising an amino acid sequence of SEQ ID NO: 251, and a PD-1-HCDR3 comprising an amino acid sequence of SEQ ID NO: 252, and the PD-1-VL comprises: a PD-1-LCDR1 comprising an amino acid sequence of SEQ ID NO: 253, a PD-1-LCDR2 comprising an amino acid sequence of SEQ ID NO: 254, and a PD-1-LCDR3 comprising an amino acid sequence of SEQ ID NO: 255.

In some embodiments, provided is the aforementioned use of a combination of the anti-ILT4 antibody and the anti-PD-1 antibody in the preparation of a medicament for treating a disease, wherein the anti-PD-1 antibody comprises a heavy chain variable region PD-1-VH and a light chain variable region PD-1-VL, wherein:

the PD-1-VH comprises an amino acid sequence of SEQ ID NO: 256, and the PD-1-VL comprises an amino acid sequence of SEQ ID NO: 257.

In some embodiments, provided is the aforementioned use of a combination of the anti-ILT4 antibody and the anti-PD-1 antibody in the preparation of a medicament for treating a disease, wherein the anti-PD-1 antibody comprises a heavy chain and a light chain, wherein:

the heavy chain comprises an amino acid sequence of SEQ ID NO: 248, and the light chain comprises an amino acid sequence of SEQ ID NO: 249.

In another aspect, provided is a combination of the anti-ILT4 antibody and the anti-PD-1 antibody according to any one of the above for use in the treatment of a disease; preferably, the disease is a tumor; more preferably, the disease is a disease selected from the group consisting of astrocytoma (e.g., anaplastic astrocytoma), glioblastoma, bladder cancer, bone cancer, brain cancer, breast cancer (e.g., breast cancer characterized by BRCA1 and/or BRCA2 mutations), carcinoid, cervical cancer, choroid plexus papilloma, colorectal cancer (e.g., colon cancer and rectal cancer), fallopian tube cancer, ependymoma, gallbladder cancer, gastric cancer, head and neck cancer, idiopathic myelofibrosis, renal cancer or renal cell carcinoma (e.g., rhabdoid tumor of the kidney and Wilms' tumor), renal pelvis tumor, leukemia, liver cancer (e.g., hepatocellular carcinoma), esophageal cancer (also known as “oesophageal cancer”, e.g., esophageal squamous cell carcinoma), lung cancer (e.g., non-small cell lung cancer and small cell lung cancer), lymphoma, medulloblastoma, melanoma, meningioma, Merkel cell carcinoma, mesothelioma, multiple myeloma, neuroblastoma, oligodendroglioma, ovarian cancer, peritoneal tumor, pancreatic cancer, polycythemia vera, primary neuroectodermal tumor, prostate cancer, retinoblastoma, salivary gland cancer, sarcoma (e.g., chondrosarcoma, Ewing sarcoma, osteosarcoma, rhabdomyosarcoma, synovial sarcoma, and soft tissue sarcoma), small intestine cancer, colorectal cancer, squamous cell carcinoma (e.g., cutaneous squamous cell carcinoma), thyroid cancer, endometrial cancer, vestibular schwannoma, blastoma, teratoma, pituitary cancer, vulva cancer, thymoma, testicular cancer, bile duct cancer, pheochromocytoma, paraganglioma, and adenoid cystic carcinoma. In some embodiments, the aforementioned medicament is used for treating a disease selected from the group consisting of ovarian cancer, lung cancer, esophageal squamous cell carcinoma, colorectal cancer, renal cell carcinoma, and melanoma.

### BRIEF DESCRIPTION OF THE DRAWINGS

FIG. 1A shows the blocking effect of the anti-ILT4 antibody huP2-E3 on ILT4 and its downstream signaling pathways.

FIG. 1B shows the blocking effect of the anti-ILT4 antibody huP2-G9 on ILT4 and its downstream signaling pathways.

FIG. 1C shows the blocking effect of the anti-ILT4 antibody huP2-C6 on ILT4 and its downstream signaling pathways.

FIG. 1D shows the blocking effect of the anti-ILT4 antibody P2m on ILT4 and its downstream signaling pathways.

FIG. 2A shows the effect of the anti-ILT4 antibody huP2-G9 on the secretion of the cytokine TNF-α by macrophages.

FIG. 2B shows the effect of the anti-ILT4 antibody huP2-C6 on the secretion of the cytokine TNF-α by macrophages.

FIG. 2C shows the effect of the anti-ILT4 antibody huP2-E8 on the secretion of the cytokine TNF-α by macrophages.

FIG. 2D shows the effect of the anti-ILT4 antibody P2m on the secretion of the cytokine TNF-α by macrophages.

FIG. 3A shows the combined effect of the anti-ILT4 antibody huP2-E3 and the anti-PD-1 antibody in allogeneic MLR.

FIG. 3B shows the combined effect of the anti-ILT4 antibody huP2-G9 and the anti-PD-1 antibody in allogeneic MLR.

FIG. 3C shows the combined effect of the anti-ILT4 antibody huP2-C6 and the anti-PD-1 antibody in allogeneic MLR.

FIG. 3D shows the combined effect of the anti-ILT4 antibody huP2-E8 and the anti-PD-1 antibody in allogeneic MLR.

FIG. 3E shows the combined effect of the anti-ILT4 antibody P2m and the anti-PD-1 antibody in allogeneic MLR.

### DETAILED DESCRIPTION

### Terminology

To facilitate the understanding of the present disclosure, some technical and scientific terms are described below. Unless otherwise specifically defined herein, all technical and scientific terms used herein have the same meaning as commonly understood by those of ordinary skill in the art.

The singular forms “a”, “an”, and “the” used in the description and claims include plural reference unless the context clearly dictates otherwise.

Unless otherwise clearly stated in the context, throughout the description and the claims, the words “comprise”, “have”, “include”, and the like, should be construed in an inclusive sense as opposed to an exclusive or exhaustive sense.

The term “cytokine” is a general term for proteins that are released by a cell population to act on other cells as intercellular mediators. Examples of such cytokines include lymphokines, monokines, chemokines, and traditional polypeptide hormones. Exemplary cytokines include: mIL-2, IFNγ, TNFα, CCL-2, and IL-6.

The term “and/or” is meant to include the two meanings “and” and “or”. For example, the phrase “A, B, and/or C” is intended to encompass each of the following: A, B, and C; A, B, or C; A or C; A or B; B or C; A and C; A and B; B and C; A (alone); B (alone); and C (alone).

The three-letter and single-letter codes for amino acids used herein are as described in J. biol. chem. 243, p3558 (1968).

The term “ILT4” refers to full-length ILT4 protein. Human ILT4 comprises an amino acid sequence of SEQ ID NO: 3. The amino acid sequences of ILT4 molecules from non-human species (e.g., mice, monkeys, rabbits, dogs, and pigs) may be obtained from public sources.

The term “αPD-1” refers to an anti-PD-1 antibody.

The term “amino acid” refers to naturally occurring and synthetic amino acids, as well as amino acid analogs and amino acid mimics that function in a manner similar to the naturally occurring amino acids. Naturally occurring amino acids are those encoded by genetic codes and those amino acids later modified, e.g., hydroxyproline, γ-carboxyglutamic acid, and O-phosphoserine. Amino acid analogs refer to compounds that have a substantially identical chemical structure (i.e., an a carbon that binds to hydrogen, carboxyl, amino, and an R group) to naturally occurring amino acids, e.g., homoserine, norleucine, methionine sulfoxide, and methioninemethyl sulfonium. Such analogs have a modified R group (e.g., norleucine) or a modified peptide skeleton, but retain a substantially identical chemical structure to naturally occurring amino acids. Amino acid mimics refer to chemical compounds that have a different structure from amino acids, but function in a manner similar to naturally occurring amino acids.

The term “amino acid mutation” includes amino acid substitutions (also known as amino acid replacements), deletions, insertions, and modifications. Any combination of substitutions, deletions, insertions, and modifications can be made to obtain a final construct, as long as the final construct possesses the desired properties, such as reduced binding to the Fc receptor. Amino acid sequence deletions and insertions include deletions and insertions at the amino terminus and/or the carboxyl terminus of a polypeptide chain. Specific amino acid mutations may be amino acid substitutions. In one embodiment, the amino acid mutation is a non-conservative amino acid substitution, i.e., the replacement of one amino acid with another amino acid having different structural and/or chemical properties. Amino acid substitutions include replacement with non-naturally occurring amino acids or with derivatives of the 20 natural amino acids (e.g., 4-hydroxyproline, 3-methylhistidine, ornithine, homoserine, and 5-hydroxylysine). Amino acid mutations can be generated using genetic or chemical methods well known in the art. Genetic methods may include site-directed mutagenesis, PCR, gene synthesis, and the like. It is contemplated that methods for altering amino acid side chain groups other than genetic engineering, such as chemical modification, may also be used. Various names may be used herein to indicate the same amino acid mutation. Herein, the expression of “position+amino acid residue” may be used to denote an amino acid residue at a specific position. For example, 102S indicates that the amino acid residue at position 102 is S. C102S indicates that the amino acid residue at position 102 is mutated from the original C into S. It should be understood that when an amino acid sequence is defined in the claims using a position+residue, the amino acid at that position before mutation does not limit the technical solutions.

The term “antibody” is used in the broadest sense and encompasses a variety of antibody structures, including but not limited to monoclonal antibodies, polyclonal antibodies; monospecific antibodies, multispecific antibodies (e.g., bispecific antibodies); and full-length antibodies, and antibody fragments (or antigen-binding fragments, or antigen-binding moieties) so long as they exhibit the desired antigen-binding activity.

“Natural antibody” refers to a naturally occurring immunoglobulin molecule. For example, a natural IgG antibody is a heterotetrameric glycoprotein of about 150,000 Daltons composed of two light chains and two heavy chains linked by a disulfide bond. From the N-terminus to the C-terminus, each heavy chain has one variable region (VH), also known as variable heavy domain or heavy chain variable region, followed by heavy chain constant regions. Generally, a natural IgG heavy chain constant region comprises three constant domains (CH1, CH2, and CH3). Similarly, from the N-terminus to the C-terminus, each light chain has one variable region (VL), also known as variable light domain or light chain variable domain, followed by one constant light domain (light chain constant region, CL). The term “full-length antibody”, “intact antibody”, and “whole antibody” are used herein interchangeably and refer to an antibody having a substantially similar structure to a natural antibody structure or having heavy chains in an Fc region as defined herein. In a natural intact antibody, the light chain comprises light chain variable region VL and constant region CL, wherein the VL is positioned at the amino terminus of the light chain, and the light chain constant region comprises a k chain and a 2 chain; the heavy chain comprises a variable region VH and a constant region (CH1, CH2, and CH3), wherein the VH is positioned at the amino terminus of the heavy chain, and the constant region is positioned at the carboxyl terminus, wherein the CH3 is closest to the carboxyl terminus of the polypeptide, and the heavy chain can be of any isotype, including IgG (including subtypes IgG1, IgG2, IgG3, and IgG4), IgA (including subtypes IgAQ1 and IgA2), IgM, and IgE. The term “variable region” or “variable domain” of an antibody refers to a domain in an antibody heavy or light chain that is involved in the binding of the antibody to an antigen. Herein, the heavy chain variable region (VH) and light chain variable region (VL) of the antibody each comprise four conserved framework regions (FRs) and three complementarity determining regions (CDRs). The term “complementarity determining region” or “CDR” refers to a region in the variable domain that primarily contributes to antigen binding; “framework” or “FR” refers to variable domain residues other than CDR residues. A VH comprises 3 CDRs: HCDR1, HCDR2, and HCDR3; a VL comprises 3 CDRs: LCDR1, LCDR2, and LCDR3. Each VH and VL is composed of three CDRs and four FRs arranged from the amino terminus (also known as N-terminus) to the carboxyl terminus (also known as C-terminus) in the following order: FR1, CDR1, FR2, CDR2, FR3, CDR3, and FR4.

The amino acid sequence boundaries of the CDRs can be determined by a variety of well-known schemes, for example, the “Kabat” numbering scheme (see Kabat et al., (1991), “Sequences of Proteins of Immunological Interest”, 5th ed., Public Health Service, National Institutes of Health, Bethesda, MD), the “Chothia” numbering scheme, the “ABM” numbering scheme, the “contact” numbering scheme (see Martin, ACR. Protein Sequence and Structure Analysis of Antibody Variable Domains [J]. 2001), and the ImMunoGen Tics (IMGT) numbering scheme (Lefranc, M. P. et al., Dev. Comp. Immunol., 27, 55-77 (2003); Front Immunol. 2018 Oct. 16; 9:2278), and the like. The corresponding relationships between the various numbering schemes are well known to those skilled in the art and, illustratively, are shown in Table 1 below.

TABLE 1
Relationships between CDR numbering schemes

| CDR | IMGT | Kabat | AbM | Chothia | Contact |
|---|---|---|---|---|---|
| HCDR1 | 27-38 | 31-35 | 26-35 | 26-32 | 30-35 |
| HCDR2 | 56-65 | 50-65 | 50-58 | 52-56 | 47-58 |
| HCDR3 | 105-117 | 95-102 | 95-102 | 95-102 | 93-101 |
| LCDR1 | 27-38 | 24-34 | 24-34 | 24-34 | 30-36 |
| LCDR2 | 56-65 | 50-56 | 50-56 | 50-56 | 46-55 |
| LCDR3 | 105-117 | 89-97 | 89-97 | 89-97 | 89-96 |

Unless otherwise stated, the “Kabat” numbering scheme is applied to the variable regions and CDRs in examples of the present disclosure. Although one numbering scheme (e.g., Kabat) is employed to define amino acid residues in specific embodiments, corresponding technical solutions for other numbering schemes are to be considered as equivalent technical solutions.

The term “antibody fragment” refers to a molecule different from an intact antibody, which comprises a moiety of an intact antibody that binds to an antigen to which the intact antibody binds. Examples of antibody fragments include, but are not limited to, Fv, Fab, Fab′, Fab′-SH, F(ab′)2, single-domain antibodies, single-chain Fab (scFab), diabodies, linear antibodies, single-chain antibodies (e.g., scFv), and multispecific antibodies formed from antibody fragments.

The term “Fc region” or “fragment crystallizable region” is used to define the C-terminal region of the heavy chain of an antibody, including native Fc regions and engineered Fc regions. In some embodiments, the Fc region comprises two subunits, which may be identical or different. In some embodiments, the Fc region of the human IgG heavy chain is defined as extending from the amino acid residue at position Cys226 or from Pro230 to its carboxyl terminus. Suitable Fc regions used for the antibodies described herein include Fc regions of human IgG1, IgG2 (IgG2A and IgG2B), IgG3, and IgG4. In some embodiments, the boundaries of the Fc region may also be varied, for example, by deleting the C-terminal lysine of the Fc region (residue 447 according to the EU numbering scheme) or deleting the C-terminal glycine and lysine of the Fc region (residues 446 and 447 according to the EU numbering scheme). Unless otherwise stated, the numbering scheme for the Fc region is the EU numbering scheme, also known as the EU index.

The term “chimeric” antibody refers to an antibody in which a portion of the heavy and/or light chain in the antibody is derived from a particular source or species, while the remainder of the heavy and/or light chain is derived from another different source or species.

The term “humanized” antibody refers to an antibody that retains the reactivity of a non-human antibody while having low immunogenicity in humans. For example, this can be achieved by retaining the non-human CDRs and replacing the remainder of the antibody with its human counterparts (i.e., the constant regions and the framework region portion of the variable regions).

The terms “human antibody”, “human-derived antibody”, “fully human antibody”, and “complete human antibody” are used interchangeably and refer to antibodies in which the variable and constant regions are human sequences. The term encompasses antibodies that are derived from human genes but have, for example, sequences that have been altered to, e.g., reduce possible immunogenicity, increase affinity, and eliminate cysteines or glycosylation sites that may cause undesired folding. The term encompasses antibodies recombinantly produced in non-human cells (that may confer glycosylation not characteristic of human cells). The term also encompasses antibodies that have been produced in transgenic mice comprising some or all of the human immunoglobulin heavy and light chain loci. The meaning of human antibodies explicitly excludes humanized antibodies.

The term “affinity” refers to the overall strength of the non-covalent interaction between a single binding site of a molecule (e.g., an antibody) and its binding ligand (e.g., an antigen). Unless otherwise indicated, as used herein, binding “affinity” refers to an internal binding affinity that reflects the 1:1 interaction between members of a binding pair (e.g., an antibody and an antigen). The affinity of a molecule X for its ligand Y can be generally denoted by the dissociation constant (KD). Affinity can be determined by conventional methods known in the art, including those described herein.

As used herein, the term “kassoc” or “ka” refers to the association rate of a particular antibody-antigen interaction, while the term “kdis” or “kd” refers to the dissociation rate of a particular antibody-antigen interaction. The term “KD” refers to the dissociation constant, which is obtained from the ratio of kd to ka (i.e., kd/ka) and denoted by a molar concentration (M). The KD value of an antibody can be determined using methods well known in the art. For example, surface plasmon resonance is determined using a biosensing system such as a system (e.g., Biacore), or affinity in a solution is determined by solution equilibrium titration (SET).

The term “surface plasmon resonance” refers to an optical phenomenon that allows for the analysis of real-time interactions by detecting changes in protein concentrations within a biosensor matrix, for example, using the BIAcore™ system (Biacore Life Sciences division of GE Healthcare, Piscataway, NJ).

The term “effector function” refers to biological activities that can be attributed to the Fc region of an antibody (either the natural sequence Fc region or the amino acid sequence variant Fc region) and vary with the antibody isotype. Examples of effector functions of an antibody include, but are not limited to: C1q binding and complement-dependent cytotoxicity, Fc receptor binding, antibody-dependent cell-mediated cytotoxicity (ADCC), phagocytosis, down-regulation of cell surface receptors (e.g., B cell receptors), and B cell activation.

The term “monoclonal antibody” refers to a population of substantially homogeneous antibodies, that is, the amino acid sequences of the antibody molecules comprised in the population are identical, except for a small number of natural mutations that may exist. In contrast, a polyclonal antibody generally comprises a plurality of different antibodies having different amino acid sequences in their variable domains, which are generally specific for different epitopes. “Monoclonal” should not be construed as requiring the production of the antibody by any particular method. In some embodiments, the antibody provided by the present disclosure is a monoclonal antibody.

The term “antigen” refers to a molecule or a portion of a molecule that can be selectively bound, for example, by an antigen-binding protein (including, for example, an antibody). An antigen may have one or more epitopes capable of interacting with different antigen-binding proteins (e.g., antibodies).

The term “epitope” refers to an area or region on an antigen that is capable of specifically binding to an antibody or an antigen-binding fragment thereof. Epitopes can be formed from contiguous strings of amino acids (linear epitope) or comprise non-contiguous amino acids (conformational epitope), e.g., coming in spatial proximity due to the folding of the antigen. The difference between the conformational epitope and the linear epitope is that in the presence of denaturing solvents, the binding of the antibody to the conformational epitope is lost. An epitope comprises at least 3, at least 4, at least 5, at least 6, at least 7, or 8-10 amino acids in a unique spatial conformation. Screening for antibodies that bind to particular epitopes (i.e., those that bind to identical epitopes) can be performed using routine methods in the art, including, for example, but not limited to, alanine scanning, peptide blotting, peptide cleavage analysis, epitope excision, epitope extraction, chemical modification of the antigen (Prot. Sci. 9 (2000) 487-496), and cross-blocking.

The term “capable of specifically binding”, “specifically binding”, or “binding” means that an antibody is capable of binding to a certain antigen or an epitope thereof with higher affinity than to other antigens or epitopes. Generally, an antibody binds to an antigen or an epitope thereof with an equilibrium dissociation constant (KD) of about 1×10⁻⁷ M or less (e.g., about 1×10⁻⁸ M, 1×10⁻⁹ M, 1×10⁻¹⁰ M, 1×10⁻¹¹ M, or less). In some embodiments, the KD for the binding of an antibody to an antigen is 10% or less (e.g., 1%) of the KD for the binding of the antibody to a non-specific antigen (e.g., BSA or casein). KD may be determined using known methods, for example, by a BIACORE® surface plasmon resonance assay. However, an antibody that specifically binds to an antigen or an epitope thereof may have cross-reactivity to other related antigens, e.g., to corresponding antigens from other species (homologous), such as humans or monkeys, e.g., Macaca fascicularis (cynomolgus, cyno), Pan troglodytes (chimpanzee, chimp), or Callithrix jacchus (commonmarmoset, marmoset).

The terms “anti-ILT4 antibody” and “antibody that binds to ILT4” refer to an antibody that is capable of binding to ILT4 or an epitope thereof with sufficient affinity.

The term “antibody-dependent cellular cytotoxicity”, “antibody-dependent cell-mediated cytotoxicity”, or “ADCC” is a mechanism for inducing cell death, which depends on the interaction of antibody-coated target cells with effector cells with lytic activity, such as natural killer cells (NK), monocytes, macrophages, and neutrophils, via an Fcγ receptor (FcγR) expressed on the effector cells. For example, NK cells express FcγRIIIa, while monocytes express FcγRI, FcγRII, and FcγRIIIa. The ADCC activity of the antibodies provided herein can be assessed by in vitro assays, using cells expressing the antigen as target cells and NK cells as effector cells. Cell lysis is detected based on the release of a label (e.g., radioactive substrate, fluorescent dye, or natural intracellular protein) from the lysed cells.

The term “antibody-dependent cellular phagocytosis (ADCP)” refers to a mechanism by which antibody-coated target cells are eliminated by internalization of phagocytic cells (such as macrophages or dendritic cells).

The term “complement-dependent cytotoxicity” or “CDC” refers to a mechanism for inducing cell death in which the Fc effector domain of a target-binding antibody binds to and activates a complement component C1q, and C1q then activates the complement cascade, resulting in the death of the target cell. Activation of a complement may also result in the deposition of complement components on the surface of target cells that promote CDC by binding to complement receptors on leukocytes (e.g., CR3).

The term “nucleic acid” is used interchangeably herein with the term “polynucleotide” and refers to deoxyribonucleotide or ribonucleotide and a polymer thereof in either single-stranded or double-stranded form. The term encompasses nucleic acids comprising known nucleotide analogs or modified backbone residues or linkages, which are synthetic, naturally occurring, and non-naturally occurring, have similar binding properties to the reference nucleic acid, and are metabolized in a manner similar to the reference nucleotide. Examples of such analogs include, but are not limited to, phosphorothioate, phosphoramidate, methylphosphonate, chiral-methylphosphonate, 2-O-methyl ribonucleotide, and peptide-nucleic acid (PNA).

“Isolated” nucleic acid refers to a nucleic acid molecule that has been separated from components of its natural environment. An isolated nucleic acid encoding a polypeptide refers to one or more nucleic acid molecules encoding the polypeptide, including such one or more nucleic acid molecules in a single vector or separate vectors, and such one or more nucleic acid molecules present at one or more positions in a host cell. Unless otherwise stated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be obtained by generating sequences in which the third position of one or more selected (or all) codons is substituted with mixed bases and/or deoxyinosine residues.

The terms “polypeptide” and “protein” are used interchangeably herein and refer to a polymer of amino acid residues. The terms apply to amino acid polymers in which one or more amino acid residues are artificial chemical mimics of corresponding naturally occurring amino acids, as well as to naturally occurring amino acid polymers and non-naturally occurring amino acid polymers. Unless otherwise stated, a particular polypeptide sequence also implicitly encompasses conservatively modified variants thereof.

The term sequence “identity” refers to the degree (percentage) to which the amino acids/nucleic acids of two sequences are identical at equivalent positions when the two sequences are optimally aligned; gaps are introduced, when necessary, to achieve the maximum percent sequence identity, and any conservative substitution is not seen as part of the sequence identity. To determine percent sequence identity, alignments can be accomplished by techniques known to those skilled in the art, for example, using publicly available computer software, such as BLAST, BLAST-2, ALIGN, ALIGN-2, or Megalign (DNASTAR) software. Those skilled in the art can determine parameters suitable for measuring alignment, including any algorithms required to achieve maximum alignment of the full length of the aligned sequences.

The term “vector” means a polynucleotide molecule capable of transporting another polynucleotide linked thereto. One type of vector is a “plasmid”, which refers to a circular double-stranded DNA loop into which additional DNA segments can be ligated. Another type of vector is a viral vector, such as an adeno-associated viral vector (AAV or AAV2), wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. The term “expression vector” or “expression construct” refers to a vector that can be transformed into a host cell and comprises a nucleic acid sequence that directs and/or controls (along with the host cell) the expression of one or more heterologous coding regions operably linked thereto. Expression constructs may include, but are not limited to, sequences that affect or control transcription and translation and affect RNA splicing of a coding region operably linked thereto in the presence of an intron.

The terms “host cell”, “host cell line”, and “host cell culture” are used interchangeably and refer to cells into which exogenous nucleic acids have been introduced, including progenies of such cells. Host cells include “transformants” and “transformed cells”, which include primary transformed cells and progeny derived therefrom, regardless of the number of passages. Progeny may not be completely identical to parent cells in terms of nucleic acid content and may contain mutations. Mutant progenies that have the same function or biological activity as the cells screened or selected from the initially transformed cells are included herein. Host cells include prokaryotic and eukaryotic host cells, wherein the eukaryotic host cells include, but are not limited to, mammalian cells, insect cell lines, plant cells, and fungal cells. Mammalian host cells include human, mouse, rat, canine, monkey, porcine, goat, bovine, equine, and hamster cells, including but not limited to, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, and HEK-293 cells. Fungal cells include yeast and filamentous fungal cells, including, for example, Pichiapastoris, Pichia finlandica, Pichia trehalophila, Pichia koclamae, Pichia membranaefaciens, Pichia minuta (Ogataea minuta, Pichia lindneri), Pichiaopuntiae, Pichia thermotolerans, Pichia salictaria, Pichia guercuum, Pichia pijperi, Pichia stiptis, Pichia methanolica, Pichia, Saccharomycescerevisiae, Saccharomyces, Hansenla polymorpha, Kluyveromyces, Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens, and Neurospora crassa. Pichia, any Saccharomyces, Hansenula polymorpha, any Kluyveromyces, Candida albicans, any Aspergillus, Trichoderma reesei, Chrysosporium lucknowense, any Fusarium, Yarrowia lipolytica, and Neurospora crassa. “Optional” or “optionally” means that the event or circumstance subsequently described may, but does not necessarily, occur, and this description includes instances where the event or circumstance occurs or does not occur.

The term “pharmaceutical composition” refers to a mixture comprising one or more of the anti-ILT4 antibodies described herein and other chemical components; the other components are, for example, physiological/pharmaceutically acceptable carriers and excipients.

The term “pharmaceutically acceptable carriers, diluents, or excipients” refers to ingredients in a pharmaceutical formulation that are different from the active ingredient and are not toxic to the subject. Pharmaceutically acceptable carriers, diluents, or excipients include, but are not limited to, buffers, excipients, stabilizers, or preservatives.

The term “subject” or “individual” includes humans and non-human animals. Non-human animals include all vertebrates (e.g., mammals and non-mammals) such as non-human primates, sheep, dogs, cows, chickens, amphibians, and reptiles. Unless indicated, the terms “patient” and “subject” are used interchangeably herein. In certain embodiments, the individual or subject is a human.

“Administrating” or “giving”, when applied to animals, humans, experimental subjects, cells, tissue, organs, or biological fluids, refers to contact of an exogenous drug, a therapeutic agent, a diagnostic agent, or a composition with the animals, humans, subjects, cells, tissue, organs, or biological fluids.

The term “sample” refers to a collection isolated from a subject (such as fluids, cells, or tissues), as well as fluids, cells, or tissues present in a subject. Exemplary samples are biological fluids (such as blood; serum; serosal fluids; plasma; lymph; urine; saliva; cystic fluids; tears; excretions; sputum; mucosal secretions of secretory tissue or organs; vaginal secretions; ascites; fluids in the pleura; pericardium; peritoneum; fluids from abdominal cavity and other body cavities; fluids collected from bronchial lavage; synovial fluids; liquid solutions in contact with a subject or biological source, e.g., culture media (including conditioned media); and lavage fluids), tissue biopsy samples, fine needle punctures, surgically excised tissues, organ cultures, or cell cultures.

“Treatment” or “treat” (and grammatical variations thereof) refers to clinical intervention applied to the treated individual, and the clinical intervention may be performed either for prophylaxis or during the course of clinical pathology. Desirable effects of the treatment include, but are not limited to, preventing the occurrence or recurrence of a disease, alleviating symptoms, alleviating/reducing any direct or indirect pathological consequences of the disease, preventing metastasis, decreasing the rate of disease progression, ameliorating or alleviating the disease state, and regressing or improving prognosis. In some embodiments, the antibody of the present disclosure is used to delay the development of or slow the progression of a disease.

“Effective amount” is generally an amount sufficient to reduce the severity and/or frequency of symptoms, eliminate symptoms and/or underlying causes, prevent the appearance of symptoms and/or their underlying causes, and/or alleviate or ameliorate damage caused by or associated with a disease state (e.g., a lung disease). In some examples, the effective amount is a therapeutically effective amount or a prophylactically effective amount.

“Therapeutically effective amount” is an amount sufficient to treat a disease state or symptom, particularly a state or symptom associated with the disease state, or to otherwise prevent, hinder, delay, or reverse the progression of the disease state or any other undesirable symptoms associated with the disease in any way. “Prophylactically effective amount” is an amount that, when administered to a subject, will have a predetermined prophylactic effect, e.g., preventing or delaying the onset (or recurrence) of the disease state, or reducing the likelihood of the onset (or recurrence) of the disease state or associated symptoms. Complete treatment or prevention does not necessarily occur after administration of one dose and may occur after administration of a series of doses. Thus, a therapeutically or prophylactically effective amount may be administered in one or more doses. “Therapeutically effective amount” and “prophylactically effective amount” may vary depending on a variety of factors such as the disease state, age, sex, and weight of the individual, and the ability of a therapeutic agent or combination of therapeutic agents to elicit a desired response in the individual. Exemplary indicators of an effective therapeutic agent or combination of therapeutic agents include, for example, improved health of a patient.

The term “immune checkpoint” refers to a set of molecules on the cell surface of CD4 T cells and CD8 T cells. These molecules can effectively act as a “brake” to down-regulate or inhibit the anti-tumor immune response. Immune checkpoint molecules include, but are not limited to, programmed death-1 (PD-1), cytotoxic T lymphocyte antigen 4 (CTLA-4), B7H1, B7H4, OX-40, CD137, CD40, and LAG-3, which directly inhibit immune cells. Immune checkpoint inhibitors for use in the present disclosure are substances that inhibit the functions of immune checkpoint molecules. There is no particular limitation on immune checkpoint inhibitors as long as the inhibitors are substances that can inhibit the functions (signals) of immune checkpoint molecules. Inhibitors that can be used as immune checkpoint inhibitors in the methods of the present disclosure include, but are not limited to, inhibitors of PD-1, PD-L2, CTLA-4, TIM-3, LAG-3, VISTA, BTLA, TIGIT, LAIR1, CD160, 2B4, CEACAM (e.g., CEACAM-1, CEACAM-3, and/or CEACAM-5), and/or TGFRβ. The inhibition of an inhibitory molecule may be performed by inhibition of a DNA, RNA, or protein level. In an embodiment, an inhibitory nucleic acid (e.g., dsRNA, siRNA, or shRNA) may be used to inhibit the expression of an inhibitory molecule. In other embodiments, an inhibitor of an inhibitory signal is a polypeptide that binds to an inhibitory molecule, e.g., a soluble ligand or antibody. Examples of the immune checkpoint inhibitors used in the present disclosure may include, but are not particularly limited to, anti-PD-1 antibodies, anti-PD-L1 antibodies, and anti-CTLA-4 antibodies, and anti-PD-1 antibodies may be preferred.

### Target Molecule

“ILT4” should be understood in the broadest sense and is intended to encompass various forms of molecules of ILT4 in various stages in mammals, such as, but not limited to, molecules produced by the ILT4 gene during amplification, replication, transcription, splicing, processing, translation, and modification, e.g., precursor ILT4, mature ILT4, ILT4 expressed on the membrane, ILT4 splice variants, modified ILT4, or fragments thereof; the term also encompasses ILT4 artificially prepared or expressed in vitro.

### Disclosed Anti-ILT4 Antibodies

The present disclosure provides anti-ILT4 antibodies having a number of advantageous properties, such as good in vitro killing activity, therapeutic activity, safety, pharmacokinetic properties, and druggability (e.g., yield, purity, and stability).

### Exemplary Anti-ILT4 Antibodies

The examples of the present disclosure disclose the antibody series P1-B4, P1-B9, P1-D10, P2-E8, P2-C10, P2-F4, P2-B3, P2-E7, P2-G4, P2-C4, P2-E11, P1-F7, P2-E3, P2-F10, P2-G9, P1-C4, P1-G7, P2-C7, P2-C6, P1-D7, P1-E11, P2-B9, P2-C2, and P2-E10. Below, the antibodies P2-G9 and P2-C6 are used as examples to describe the antibodies of the present disclosure.

Illustratively, provided is an anti-ILT4 antibody of the present disclosure, wherein the anti-ILT4 antibody comprises a heavy chain variable region and a light chain variable region, wherein:
a. the HCDR1, the HCDR2, and the HCDR3 of the heavy chain variable region comprise amino acid sequences of a HCDR1, a HCDR2, and a HCDR3, respectively, in SEQ ID NO: 184 or 185, and the LCDR1, the LCDR2, and the LCDR3 of the light chain variable region comprise amino acid sequences of a LCDR1, a LCDR2, and a LCDR3, respectively, in SEQ ID NO: 187 or 186; or the HCDR1, the HCDR2, and the HCDR3 of the heavy chain variable region comprise amino acid sequences of a HCDR1, a HCDR2, and a HCDR3, respectively, in SEQ ID NO: 32, and the LCDR1, the LCDR2, and the LCDR3 of the light chain variable region comprise amino acid sequences of a LCDR1, a LCDR2, and a LCDR3, respectively, in SEQ ID NO: 33; or
b. the HCDR1, the HCDR2, and the HCDR3 of the heavy chain variable region comprise amino acid sequences of a HCDR1, a HCDR2, and a HCDR3, respectively, in any one of SEQ ID NO: 211, 205, 206, 207, 208, 209, or 210, and the LCDR1, the LCDR2, and the LCDR3 of the light chain variable region comprise amino acid sequences of a LCDR1, a LCDR2, and a LCDR3, respectively, in any one of SEQ ID NO: 212, 213, or 214; or the HCDR1, the HCDR2, and the HCDR3 of the heavy chain variable region comprise amino acid sequences of a HCDR1, a HCDR2, and a HCDR3, respectively, in SEQ ID NO: 40, and the LCDR1, the LCDR2, and the LCDR3 of the light chain variable region comprise amino acid sequences of a LCDR1, a LCDR2, and a LCDR3, respectively, in SEQ ID NO: 41.

Illustratively, provided is an anti-ILT4 antibody of the present disclosure, wherein the anti-ILT4 antibody comprises a heavy chain variable region and a light chain variable region, wherein:
a. the HCDR1, the HCDR2, and the HCDR3 of the heavy chain variable region comprise amino acid sequences of a HCDR1, a HCDR2, and a HCDR3, respectively, in SEQ ID NO: 184, and the LCDR1, the LCDR2, and the LCDR3 of the light chain variable region comprise amino acid sequences of a LCDR1, a LCDR2, and a LCDR3, respectively, in SEQ ID NO: 187; or
b. the HCDR1, the HCDR2, and the HCDR3 of the heavy chain variable region comprise amino acid sequences of a HCDR1, a HCDR2, and a HCDR3, respectively, in SEQ ID NO: 211, and the LCDR1, the LCDR2, and the LCDR3 of the light chain variable region comprise amino acid sequences of a LCDR1, a LCDR2, and a LCDR3, respectively, in SEQ ID NO: 212.

Illustratively, provided is an anti-ILT4 antibody of the present disclosure, wherein the HCDR1, the HCDR2, and the HCDR3 of the heavy chain variable region and the LCDR1, the LCDR2, and the LCDR3 of the light chain variable region are defined according to the same numbering scheme selected from the group consisting of Kabat, IMGT, Chothia, AbM, and Contact.

Illustratively, provided is an anti-ILT4 antibody of the present disclosure, wherein the anti-ILT4 antibody comprises a heavy chain variable region and a light chain variable region, wherein:

in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119.

Illustratively, provided is an anti-ILT4 antibody of the present disclosure, wherein the anti-ILT4 antibody comprises a heavy chain variable region and a light chain variable region, wherein:

in the heavy chain variable region, the HCDR1 comprises an amino acid sequence of SEQ ID NO: 135, the HCDR2 comprises an amino acid sequence of SEQ ID NO: 203, 136, 201, 202, or 204, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140;

preferably, in the heavy chain variable region, the HCDR1 comprises an amino acid sequence of SEQ ID NO: 135, the HCDR2 comprises an amino acid sequence of SEQ ID NO: 203, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140.

Illustratively, provided is an anti-ILT4 antibody of the present disclosure, wherein the HCDR1, the HCDR2, and the HCDR3 of the heavy chain variable region and the LCDR1, the LCDR2, and the LCDR3 of the light chain variable region are defined according to the Kabat numbering scheme.

Illustratively, provided is an anti-ILT4 antibody of the present disclosure, wherein:
a. in the heavy chain variable region, the HCDR1 comprises an amino acid sequence of SEQ ID NO: 260, the HCDR2 comprises an amino acid sequence of SEQ ID NO: 261, and the HCDR3 comprises an amino acid sequence of SEQ ID NO: 262; and in the light chain variable region, the LCDR1 comprises an amino acid sequence of SEQ ID NO: 263, the LCDR2 comprises an amino acid sequence of SEQ ID NO: 264, and the LCDR3 comprises an amino acid sequence of SEQ ID NO: 119; or
b. in the heavy chain variable region, the HCDR1 comprises an amino acid sequence of SEQ ID NO: 267, the HCDR2 comprises an amino acid sequence of SEQ ID NO: 268, and the HCDR3 comprises an amino acid sequence of SEQ ID NO: 269; and in the light chain variable region, the LCDR1 comprises an amino acid sequence of SEQ ID NO: 270, the LCDR2 comprises an amino acid sequence of SEQ ID NO: 271, and the LCDR3 comprises an amino acid sequence of SEQ ID NO: 140;
wherein the CDRs are defined according to the IMGT numbering scheme.

Illustratively, provided is an anti-ILT4 antibody of the present disclosure, wherein: in the heavy chain variable region, the HCDR1 comprises an amino acid sequence set forth in SEQ ID NO: 260, the HCDR2 comprises an amino acid sequence set forth in SEQ ID NO: 261, and the HCDR3 comprises an amino acid sequence set forth in SEQ ID NO: 262; and in the light chain variable region, the LCDR1 comprises an amino acid sequence set forth in SEQ ID NO: 263, the LCDR2 comprises an amino acid sequence set forth in SEQ ID NO: 264, and the LCDR3 comprises an amino acid sequence set forth in SEQ ID NO: 119;
wherein the CDRs are defined according to the IMGT numbering scheme.

Illustratively, provided is an anti-ILT4 antibody of the present disclosure, wherein:
a. in the heavy chain variable region, the HCDR1 comprises an amino acid sequence of SEQ ID NO: 265, the HCDR2 comprises an amino acid sequence of SEQ ID NO: 266, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119; or
b. in the heavy chain variable region, the HCDR1 comprises an amino acid sequence of SEQ ID NO: 272, the HCDR2 comprises an amino acid sequence of SEQ ID NO: 273, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140;
wherein the CDRs are defined according to the Chothia numbering scheme.

Illustratively, provided is an anti-ILT4 antibody of the present disclosure, wherein: in the heavy chain variable region, the HCDR1 comprises an amino acid sequence set forth in SEQ ID NO: 265, the HCDR2 comprises an amino acid sequence set forth in SEQ ID NO: 266, and the HCDR3 comprises an amino acid sequence set forth in SEQ ID NO: 121; and in the light chain variable region, the LCDR1 comprises an amino acid sequence set forth in SEQ ID NO: 114, the LCDR2 comprises an amino acid sequence set forth in SEQ ID NO: 109, and the LCDR3 comprises an amino acid sequence set forth in SEQ ID NO: 119;
wherein the CDRs are defined according to the Chothia numbering scheme.

Illustratively, provided is an anti-ILT4 antibody of the present disclosure, wherein the anti-ILT4 antibody is a murine antibody, a chimeric antibody, a humanized antibody, or a fully human-derived antibody. In some embodiments, the antibody is a chimeric antibody. In some embodiments, the antibody is a fully human-derived antibody. In some embodiments, the antibody is a humanized antibody.

Illustratively, provided is an anti-ILT4 antibody of the present disclosure, wherein the anti-ILT4 antibody comprises a framework region (FR) of a human antibody.

Illustratively, provided is an anti-ILT4 antibody of the present disclosure, wherein:
a. the heavy chain variable region comprises an amino acid sequence having at least 70% (e.g., at least 70%, 80%, 85%, 90%, 95%, 98%, or 99%) sequence identity to SEQ ID NO: 184, and the light chain variable region comprises an amino acid sequence having at least 70% (e.g., at least 70%, 80%, 85%, 90%, 95%, 98%, or 99%) sequence identity to SEQ ID NO: 187; or
b. the heavy chain variable region comprises an amino acid sequence having at least 70% (e.g., at least 70%, 80%, 85%, 90%, 95%, 98%, or 99%) sequence identity to SEQ ID NO: 211, and the light chain variable region comprises an amino acid sequence having at least 70% (e.g., at least 70%, 80%, 85%, 90%, 95%, 98%, or 99%) sequence identity to SEQ ID NO: 212.

Illustratively, provided is an anti-ILT4 antibody of the present disclosure, wherein the heavy chain variable region comprises a FR1, a FR2, and a FR3 that are derived from IGHV4-31*01 and a FR4 derived from IGHJ1*01, and the FRs are unsubstituted or have one or more amino acid substitutions selected from the group consisting of 1E, 27S, 30T, 44K, 71R, and 76S; and/or the light chain variable region comprises a FR1, a FR2, and a FR3 that are derived from IGKV1-39*01 and a FR4 derived from IGKJ4*01, and the FRs are unsubstituted or have one or more amino acid substitutions selected from the group consisting of 43S, 48V, and 71Y. In some embodiments, provided is the anti-ILT4 antibody, wherein in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121, and the FRs of the heavy chain variable region comprise one or more amino acid substitutions selected from the group consisting of 1E, 27S, 30T, 44K, 71R, and 76S; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119, and the FRs of the light chain variable region comprise one or more amino acid substitutions selected from the group consisting of 43S, 48V, and 71Y. In some embodiments, the variable regions and CDRs described above are defined according to the Kabat numbering scheme.

Illustratively, provided is an anti-ILT4 antibody of the present disclosure, wherein the heavy chain variable region comprises a FR1, a FR2, and a FR3 that are derived from IGHV1-18*01 and a FR4 derived from IGHJ6*01, and the FRs are unsubstituted or have one or more amino acid substitutions selected from the group consisting of 1E, 43E, 69L, 71V, 73K, 76N, and 93T; and/or the light chain variable region comprises a FR1, a FR2, and a FR3 that are derived from IGKV4-1*01 or IGKV3-11*01 and a FR4 derived from IGKJ4*01, and the FRs are unsubstituted or have one or more amino acid substitutions selected from the group consisting of 4L, 43P, 45K, 581, 68R, and 85T. In some embodiments, provided is the anti-ILT4 antibody, wherein in the heavy chain variable region, the HCDR1 comprises an amino acid sequence of SEQ ID NO: 135, the HCDR2 comprises an amino acid sequence of SEQ ID NO: 203, 136, 201, 202, or 204, and the HCDR3 comprises an amino acid sequence of SEQ ID NO: 137, and the FRs of the heavy chain variable region comprise one or more amino acid substitutions selected from the group consisting of 1E, 43E, 69L, 71V, 73K, 76N, and 93T; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140, and the FRs of the light chain variable region comprise one or more amino acid substitutions selected from the group consisting of 4L, 43P, 45K, 581, 68R, and 85T. In some embodiments, the variable regions and CDRs described above are defined according to the Kabat numbering scheme.

Illustratively, provided is an anti-ILT4 antibody of the present disclosure, wherein the anti-ILT4 antibody is an antibody fragment; in some embodiments, the antibody fragment is selected from the group consisting of Fab, Fab′, F(ab′)2, Fd, Fv, scFv, dsFv, and dAb.

Illustratively, provided is an anti-ILT4 antibody of the present disclosure, wherein the anti-ILT4 antibody comprises a heavy chain constant region and a light chain constant region. In some embodiments, the heavy chain constant region is a human IgG1 or IgG4 heavy chain constant region. In some embodiments, the heavy chain constant region comprises 234A and 235A mutations; the mutations are numbered according to the EU index. In some embodiments, the light chain constant region is a human k light chain constant region. In some embodiments, the heavy chain constant region comprises an amino acid sequence of SEQ ID NO: 240 or 171, and the light chain constant region comprises an amino acid sequence of SEQ ID NO: 172.

Illustratively, provided is an anti-ILT4 antibody of the present disclosure, wherein the anti-ILT4 antibody comprises a heavy chain and a light chain, wherein:
a. the heavy chain comprises an amino acid sequence having at least 70% (e.g., at least 70%, 80%, 85%, 90%, 95%, 98%, or 99%) sequence identity to SEQ ID NO: 228, and the light chain comprises an amino acid sequence having at least 70% (e.g., at least 70%, 80%, 85%, 90%, 95%, 98%, or 99%) sequence identity to SEQ ID NO: 229; or
b. the heavy chain comprises an amino acid sequence having at least 70% (e.g., at least 70%, 80%, 85%, 90%, 95%, 98%, or 99%) sequence identity to SEQ ID NO: 234, and the light chain comprises an amino acid sequence having at least 70% (e.g., at least 70%, 80%, 85%, 90%, 95%, 98%, or 99%) sequence identity to SEQ ID NO: 235.

Illustratively, provided is an anti-ILT4 antibody of the present disclosure, wherein the anti-ILT4 antibody comprises a heavy chain and a light chain, wherein:
a. the heavy chain comprises an amino acid sequence of SEQ ID NO: 228, and the light chain comprises an amino acid sequence of SEQ ID NO: 229; or
b. the heavy chain comprises an amino acid sequence of SEQ ID NO: 234, and the light chain comprises an amino acid sequence of SEQ ID NO: 235.

Illustratively, the present disclosure further provides an isolated anti-ILT4 antibody that competes for binding to human ILT4 or an epitope thereof with the anti-ILT4 antibody according to any one of the foregoing.

Illustratively, the anti-ILT4 antibody provided by the present disclosure specifically binds to human ILT4 and does not bind to other LILR family members: ILT2, ILT3, LILRB5, LILRA1, LILRA2, LILRA3, LILRA4, LILRA5, and/or LILRA6.

Illustratively, the ability of the isolated anti-ILT4 antibody of the present disclosure to bind to ILT4 protein is higher than that of a control antibody. Specifically, the isolated anti-ILT4 antibody of the present disclosure binds to human ILT4 with a KD value of less than 1×10⁻⁸ M (e.g., less than 8×10⁻⁹ M, less than 5×10⁻⁹ M, less than 2×10⁻⁹ M, less than 1×10⁻⁹ M, less than 8×10⁻¹⁰ M, less than 5×10⁻¹⁰ M, less than 2×10⁻¹⁰ M, less than 1×10⁻¹⁰ M, less than 8×10⁻¹¹ M, or less than 5×10⁻¹¹ M), as measured by surface plasmon resonance (Biacore).

Illustratively, a humanized anti-ILT4 antibody provided by the present disclosure is capable of effectively removing the inhibitory effect of ILT4 and promoting the secretion of higher levels of mIL-2 by 3A9 cells.

Illustratively, a humanized anti-ILT4 antibody provided by the present disclosure is capable of effectively promoting the transformation of macrophages to the M1 phenotype and the secretion of high levels of the proinflammatory cytokine TNF-α.

Illustratively, a humanized anti-ILT4 antibody provided by the present disclosure (e.g., huP2-G9) is capable of inducing dendritic cells to secrete higher levels of proinflammatory cytokines or chemokines in an immunosuppressive environment with IL-10.

Illustratively, a humanized anti-ILT4 antibody provided by the present disclosure is capable of effectively blocking the binding of ILT4 to HLA-G.

Illustratively, a humanized anti-ILT4 antibody provided by the present disclosure is capable of effectively blocking the binding of ILT4 to HLA-G, and the blocking activity of the humanized anti-ILT4 antibody is stronger than that of the control antibodies MK and JTX.

### Antibody Structure

In certain embodiments, the antibody provided herein is a full-length antibody.

In certain embodiments, the antibody provided herein is an antibody fragment.

In one embodiment, the antibody fragment is a Fab, Fab′, Fab′-SH, or F(ab′)₂ fragment, particularly a Fab fragment. “Fab” is a monovalent fragment consisting of the VL, VH, CL, and CH1 domains. “Fab fragment” may be produced by papain cleavage of an antibody. “Fab” comprises VL, CL, and VH and CH1, and further comprises the region between the CH1 and CH2 domains, so that an interchain disulfide bond can be formed between the two heavy chains of two Fab′ fragments to form a F(ab′) 2 molecule. “Fab′-SH” is a Fab′ fragment in which the cysteine residues of the constant regions have a free sulfhydryl group. “F(ab′)2” is a bivalent fragment comprising two Fab fragments linked by a disulfide bond at the hinge region.

In another embodiment, the antibody fragment is a diabody, a triabody, or a tetrabody. Diabodies are antibody fragments having two antigen-binding sites. The fragment comprises a VH and a VL linked in the same polypeptide chain (VH-VL). By using a linker that is too short to allow pairing between two domains on the same chain, the domains are forced to pair with the complementary domains of another chain, thereby resulting in two antigen-binding sites. The two antigens may be identical or different.

In another embodiment, the antibody fragment is a single-chain Fab fragment. “Single-chain Fab fragment” or “scFab” is a polypeptide consisting of VH, CH1, VL, CL, and a linker, wherein the antibody domains and the linker have one of the following orders in the N-terminus to C-terminus direction: a) VH-CH1-linker-VL-CL, b) VL-CL-linker-VH-CH1, c) VH-CL-linker-VL-CH1, or d) VL-CH1-linker-VH-CL. In one embodiment, the linker is a polypeptide having at least 30 amino acids. In another embodiment, the linker is a polypeptide having between 32 and 50 amino acids. The single-chain Fab fragment is stabilized via the natural disulfide bond between CL and CH1. In addition, these single-chain Fab molecules may be further stabilized by generating interchain disulfide bonds through insertion of cysteine residues (e.g., position 44 in the heavy chain variable region and position 100 in the light chain variable region, according to Kabat numbering). In another embodiment, the antibody fragment is an Fv fragment consisting of the VH and VL domains of a single arm of the antibody.

In another embodiment, the antibody fragment is a single-chain variable fragment (scFv). “scFv” is a fusion protein comprising at least one antibody fragment comprising a light chain variable region and at least one antibody fragment comprising a heavy chain variable region, wherein the light and heavy chain variable regions are linked by a short flexible peptide linker and are capable of being expressed as a single-chain polypeptide, and wherein the scFv retains the specificity of the intact antibody from which it is derived. Unless specifically indicated, an scFv may have VL and VH variable regions in any order herein; for example, with respect to the N-terminus and C-terminus of the polypeptide, the scFv may comprise VL-linker-VH or may comprise VH-linker-VL.

In another embodiment, the antibody fragment is a dsFv obtained by linking polypeptides in which one amino acid residue of each of VH and VL is substituted with a cysteine residue by a disulfide bond between the cysteine residues. The amino acid residues substituted with cysteine residues can be selected according to known methods (Protein Engineering, 7:697 (1994)) based on prediction of the three-dimensional structure of the antibody.

In another embodiment, the antibody fragment is a single-domain antibody (dAb). Single-domain antibodies are antibody fragments comprising all or a portion of the heavy chain variable domain or all or a portion of the light chain variable domain of an antibody. In certain embodiments, the antibody provided herein is a chimeric antibody. In one example, a chimeric antibody comprises a non-human variable region (e.g., a variable region derived from a mouse, rat, hamster, domestic rabbit, or non-human primate, such as a monkey) and a human constant region. In another example, a chimeric antibody is a “class switched” antibody in which the class or subclass has been changed from the class or subclass of the parent antibody.

In certain embodiments, the antibody is a humanized antibody. Typically, non-human antibodies are humanized to reduce immunogenicity to humans while retaining the specificity and affinity of the parent non-human antibody. Generally, the humanized antibody comprises one or more variable regions in which the CDRs or portions thereof are derived from a non-human antibody and the FRs or portions thereof are derived from a human antibody. Optionally, the humanized antibody can further comprise a portion of a human constant region. In some embodiments, some FR residues in a humanized antibody may be replaced with corresponding residues from a non-human antibody (e.g., an antibody that provides CDR sequences).

Humanized antibodies and methods for their production are reviewed, e.g., in Almagro and Fransson, Front. Biosci. 13:1619-1633 (2008), and further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity-determining region (SDR) grafting); Padlan, Mol. Immunol. 28:489-498 (1991) (describing “resurfuacing”); Dall'Acqua et al., Methods 36:43-60 (2005) (describing “FR shuffling”); and Osbourn et al., Methods 36:61-68 (2005) and Klimka et al., Br. J. Cancer 83:252-260 (2000) (describing the “guided selection” method for FR shuffling).

Human framework regions that can be used for humanization include, but are not limited to: framework regions selected using the “best-fit” method (see, e.g., Sims et al., J. Immunol. 151:2296 (1993)); framework regions derived from the consensus sequence of human antibodies of a particular subgroup of light or heavy chain variable regions (see, e.g., Carter et al., Proc. Natl. Acad. Sci. USA, 89:4285 (1992); and Presta et al., J. Immunol., 151:2623 (1993)); human mature (somatically mutated) framework regions or human germline framework regions (see, e.g., Almagro and Fransson, Front. Biosci. 13:1619-1633 (2008)); and framework regions obtained by screening FR libraries (see, e.g., Baca et al., J. Biol. Chem. 272:10678-10684 (1997) and Rosok et al., J. Biol. Chem. 271:22611-22618 (1996)).

### Variants of Anti-ILT4 Antibody

In certain embodiments, amino acid sequence variants of the anti-ILT4 antibody provided herein are encompassed. For example, it may be desirable to improve the binding affinity and/or other biological properties of the antibody. Amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequence of the anti-ILT4 antibody. Any combination of deletion, insertion, and substitution can be made to obtain the final construct, provided that the final construct possesses the desired characteristics, e.g., antigen binding.

### Substitution, Insertion, and Deletion Variants

In certain embodiments, antibody variants having one or more amino acid substitutions are provided. Positions of interest for substitutional mutagenesis include CDRs and FRs. Conservative substitutions are shown in Table 2 under the heading of “Preferred substitution”. More substantial changes are provided in Table 2 under the heading of “exemplary substitution”, and as further described below with reference to amino acid side chain classes. Amino acid substitutions can be introduced into an antibody of interest and the products are screened for a desired activity, e.g., retained/improved antigen binding, reduced immunogenicity, or improved ADCC or CDC.

TABLE 2
Amino acid substitutions

| Original | Exemplary | Preferred |
|---|---|---|
| residue | substitution | substitution |
| Ala(A) | Val, Leu, Ile | Val |
| Arg(R) | Lys, Gln, Asn | Lys |
| Asn(N) | Gln, His, Asp, Lys, Arg | Gln |
| Asp(D) | Glu, Asn | Glu |
| Cys(C) | Ser, Ala | Ser |
| Gln(Q) | Asn, Glu | Asn |
| Glu(E) | Asp, Gln | Asp |
| Gly(G) | Ala | Ala |
| His(H) | Asn, Gln, Lys, Arg | Arg |
| Ile(I) | Leu; Val; Met; Ala; Phe; norleucine | Leu |
| Leu(L) | Norleucine; Ile; Val; Met; Ala; Phe | Ile |
| Lys(K) | Arg, Gln, Asn | Arg |
| Met(M) | Leu, Phe, Ile | Leu |
| Phe(F) | Trp, Leu, Val, Ile, Ala, Tyr | Tyr |
| Pro(P) | Ala | Ala |
| Ser(S) | Thr | Thr |
| Thr(T) | Ser | Ser |
| Trp(W) | Tyr, Phe | Tyr |
| Tyr(Y) | Trp, Phe, Thr, Ser | Phe |
| Val(V) | Ile; Leu; Met; Phe; Ala; norleucine | Leu |

According to common side-chain properties, amino acids can be grouped as follows:
(1) hydrophobic: norleucine, Met, Ala, Val, Leu, Ile;
(2) neutral, hydrophilic: Cys, Ser, Thr, Asn, Gln;
(3) acidic: Asp, Glu;
(4) basic: His, Lys, Arg;
(5) residues affecting chain orientation: Gly, Pro;
(6) aromatic: Trp, Tyr, Phe.

Non-conservative substitutions will involve substituting a member of one of these classes for a member of another class.

One type of substitutional variant involves substituting one or more CDR residues of a parent antibody (e.g., a humanized or human antibody). Generally, the resulting variant selected for further study will have changes (e.g., improvements) in certain biological properties (e.g., increased affinity or reduced immunogenicity) relative to the parent antibody, and/or will have substantially retained certain biological properties of the parent antibody. An exemplary substitutional variant is an affinity-matured antibody, which can be conveniently produced, for example, using phage display-based affinity maturation techniques such as those described herein. Briefly, one or more CDR residues are mutated and the variant antibodies are displayed on phages and screened for a particular biological activity (e.g. binding affinity). Changes (e.g., substitutions) can be made in CDRs, for example, to improve antibody affinity. Such changes can be made in CDR “hotspots”, i.e., residues encoded by codons that undergo mutation at high frequency during the somatic maturation process, and/or antigen-contacting residues, and meanwhile, the resulting variant VH or VL is tested for binding affinity. In some embodiments of affinity maturation, diversity is introduced into the variable genes selected for maturation by any one of a variety of methods (e.g., error-prone PCR, chain shuffling, or oligonucleotide-directed mutagenesis). A secondary library is then created. The library is then screened to identify any antibody variants with the desired affinity. Another method for introducing diversity involves CDR-directed methods in which several CDR residues (e.g., 4-6 residues at a time) are randomized. CDR residues involved in antigen binding can be specifically identified, for example, using alanine scanning mutagenesis or modeling. Particularly, HCDR3 and LCDR3 are often targeted.

In certain embodiments, substitutions, insertions or deletions may occur within one or more CDRs, as long as such changes do not substantially reduce the ability of the antibody to bind to the antigen. For example, conservative changes (e.g., conservative substitutions as provided herein) that do not substantially reduce binding affinity may be made in CDRs.

Such changes may be, for example, outside of the antigen-contacting residues in CDRs. In certain embodiments of the variant VH and VL sequences provided above, each CDR is unaltered or contains no more than 1, 2, or 3 amino acid substitutions.

A useful method for identifying residues or regions of an antibody that can be used as mutagenesis targets is called “alanine scanning mutagenesis”. In this method, a residue or group of target residues (e.g., charged residues such as Arg, Asp, His, Lys, and Glu) are identified and replaced with a neutral or negatively charged amino acid (e.g., Ala or polyalanine) to determine whether the interaction of the antibody with the antigen is affected. Further substitutions may be introduced at amino acid locations that show functional sensitivity to the initial substitutions. In addition, a crystal structure of an antigen-antibody complex may be studied to identify contact points between the antibody and antigen. These contact residues and neighboring residues may be targeted or eliminated as substitution candidates. Variants may be screened to determine whether they contain the desired properties.

Amino acid sequence insertions include amino- and/or carboxyl-terminal fusions ranging in length from 1 residue to polypeptides containing 100 or more residues, and intrasequence insertions of single or multiple amino acid residues. Examples of terminal insertions include an antibody with an N-terminal methionyl residue. Other insertional variants of the antibody molecule include the fusion of the N- or C-terminus of the antibody to an enzyme or a polypeptide that increases the serum half-life of the antibody.

### Recombination Method

Anti-ILT4 antibodies can be produced using recombinant methods. For these methods, one or more isolated nucleic acids encoding the anti-ILT4 antibodies are provided.

In one embodiment, the present disclosure provides an isolated nucleic acid encoding the aforementioned anti-ILT4 antibody. Such nucleic acids may each independently encode any one of the aforementioned polypeptide chains. In another aspect, the present disclosure provides one or more vectors (e.g., expression vectors) comprising such nucleic acids. In another aspect, the present disclosure provides a host cell comprising such nucleic acids. In one embodiment, provided is a method for preparing a polypeptide, wherein the method comprises culturing a host cell comprising a nucleic acid encoding the polypeptide, as provided above, under conditions suitable for expression, and optionally recovering the anti-ILT4 antibody from the host cell (or a host cell medium).

For recombinant production of the anti-ILT4 antibody, the nucleic acid encoding the protein is isolated and inserted into one or more vectors for further cloning and/or expression in a host cell. Such nucleic acids can be readily isolated and sequenced using conventional procedures, or produced by recombination methods or obtained by chemical synthesis. Suitable host cells for cloning or expressing a vector encoding the anti-ILT4 antibody include prokaryotic or eukaryotic cells described herein. For example, it can be produced in bacteria, particularly when glycosylation and Fc effector functions are not needed. After expression, it can be isolated from the bacterial cell paste in a soluble fraction and can be further purified.

In addition to prokaryotes, eukaryotic microorganisms such as filamentous fungi or yeast are suitable cloning or expression hosts for vectors encoding proteins, including fungal and yeast strains. Suitable host cells for protein expression may also be derived from multicellular organisms (invertebrates and vertebrates); examples of invertebrate cells include plant and insect cells. A number of baculovirus strains have been identified, which can be used in combination with insect cells, particularly for transfection of Spodoptera frugiperda cells; plant cell cultures may also be used as hosts, see, e.g., U.S. Pat. Nos. 5,959,177, 6,040,498, 6,420,548, 7,125,978, and 6,417,429; vertebrate cells can also be used as hosts, e.g., mammalian cell lines adapted for growth in a suspension. Other examples of suitable mammalian host cell lines include an SV40-transformed monkey kidney CV1 line (COS-7); a human embryonic kidney line (293 or 293T cells); baby hamster kidney cells (BHK); mouse sertoli cells (TM4 cells); monkey kidney cells (CV1); African green monkey kidney cells (VERO-76); human cervical cancer cells (HELA); canine kidney cells (MDCK); buffalo rat liver cells (BRL3A); human lung cells (W138); human liver cells (Hep G2); mouse mammary tumor cells (MMT 060562); TRI cells; MRC 5 cells; and FS4 cells. Other suitable mammalian host cell lines include Chinese hamster ovary (CHO) cells, including DHFR-CHO cells; and myeloma cell lines such as YO, NSO, and Sp2/0. For reviews of certain mammalian host cell lines suitable for antibody production see, e.g., Yazaki, P. and Wu, A. M., Methods in Molecular Biology, Vol. 248, Lo, B.K.C. (eds.), Humana Press, Totowa, NJ (2004), pp. 255-268.

### Assays

The physical/chemical characteristics and/or biological activity of the anti-ILT4 antibody provided herein may be identified, screened, or characterized through a variety of assays known in the art. In one aspect, the activity of the anti-ILT4 antibody of the present disclosure is tested, for example, by known methods such as ELISA and western blot.

### Treatment Method and Route of Administration

Any of the anti-ILT4 antibodies provided by the present disclosure can be used in a treatment method. In yet another aspect, the present disclosure provides use of the anti-ILT4 antibody in the manufacture or preparation of a medicament. In some embodiments, the disease is an ILT4-associated disease or disorder. In some embodiments, the disease is selected from the group consisting of astrocytoma (e.g., anaplastic astrocytoma), glioblastoma, bladder cancer, bone cancer, brain cancer, breast cancer (e.g., breast cancer characterized by BRCA1 and/or BRCA2 mutations), carcinoid, cervical cancer, choroid plexus papilloma, colorectal cancer (e.g., colon cancer and rectal cancer), fallopian tube cancer, ependymoma, gallbladder cancer, gastric cancer, head and neck cancer, idiopathic myelofibrosis, renal cancer or renal cell carcinoma (e.g., rhabdoid tumor of the kidney and Wilms' tumor), renal pelvis tumor, leukemia, liver cancer (e.g., hepatocellular carcinoma), esophageal cancer (e.g., also known as “oesophageal cancer”, esophageal squamous cell carcinoma), lung cancer (e.g., non-small cell lung cancer and small cell lung cancer), lymphoma, medulloblastoma, melanoma, meningioma, Merkel cell carcinoma, mesothelioma, multiple myeloma, neuroblastoma, oligodendroglioma, ovarian cancer, peritoneal tumor, pancreatic cancer, polycythemia vera, primary neuroectodermal tumor, prostate cancer, retinoblastoma, salivary gland cancer, sarcoma (e.g., chondrosarcoma, Ewing sarcoma, osteosarcoma, rhabdomyosarcoma, synovial sarcoma, and soft tissue sarcoma), small intestine cancer, colorectal cancer, squamous cell carcinoma (e.g., cutaneous squamous cell carcinoma), thyroid cancer, endometrial cancer, vestibular schwannoma, blastoma, teratoma, pituitary cancer, vulva cancer, thymoma, testicular cancer, bile duct cancer, pheochromocytoma, paraganglioma or adenoid cystic carcinoma, and thrombocytosis; in some embodiments, the disease is selected from the group consisting of ovarian cancer, lung cancer, esophageal squamous cell carcinoma, colorectal cancer, renal cell carcinoma, and melanoma.

In yet another aspect, provided is a pharmaceutical composition comprising the anti-ILT4 antibody, e.g., for use in any one of the above pharmaceutical uses or treatment methods. In one embodiment, the pharmaceutical composition comprises any of the anti-ILT4 antibodies provided herein and a pharmaceutically acceptable carrier. In another embodiment, the pharmaceutical composition further comprises at least one additional therapeutic agent.

The anti-ILT4 antibody of the present disclosure may be used alone or in combination with other agents for treatment. For example, the antibody of the present disclosure can be co-administered with at least one additional therapeutic agent.

The anti-ILT4 antibody of the present disclosure (and any additional therapeutic agent) may be administered by any suitable means, including parenteral, intrapulmonary, and intranasal administration, and, if required for local treatment, intralesional administration. Parenteral infusion includes intramuscular, intravenous, intra-arterial, intraperitoneal, or subcutaneous administration. Administration may be performed by any suitable route, e.g., by injection, such as intravenous or subcutaneous injection, depending in part on whether the administration is short-term or long-term. Various administration schedules are contemplated herein, including but not limited to, single administration or multiple administrations at multiple time points, bolus administration, and pulse infusion. The anti-ILT4 antibody of the present disclosure will be formulated, administered, and applied in a manner consistent with Good Medical Practice. Factors considered in this context include the specific disorder being treated, the specific mammal being treated, the clinical state of the individual patient, the cause of the disorder, the site of delivery of the agent, the method of administration, the timing of administration, and other factors known to medical practitioners. The anti-ILT4 antibody may be formulated with or without one or more agents currently used for preventing or treating the disorder. The effective amount of such additional agents depends on the amount present in the pharmaceutical composition, the type of the disorder or treatment, and other factors. These are generally used in the same dosages and routes of administration as described herein, or in about 1% to 99% of the dosages described herein, or in other dosages, and by any route empirically/clinically determined to be appropriate.

For the prevention or treatment of disease, the appropriate dosage of the anti-ILT4 antibody of the present disclosure (when used alone or in combination with one or more other additional therapeutic agents) will depend on the type of the disease to be treated, the type of the therapeutic molecule, the severity and course of the disease, whether the therapeutic molecule is administered for preventive or therapeutic purposes, previous therapy, the patient's clinical history and response to the therapeutic molecule, and the discretion of the attending physician. The therapeutic molecule is suitably administered to a patient in one or a series of treatments.

### Product

In another aspect of the present disclosure, provided is a product (as an example, the product is prepared in the form of a kit) comprising materials useful for the treatment, prevention, and/or diagnosis of the disorders described above. The product comprises a container and a label or package insert on or associated with the container. Suitable containers include, for example, bottles, vials, syringes, IV solution bags, and the like. The container may be formed from a variety of materials such as glass or plastic. The container contains a composition effective in the treatment, prevention, and/or diagnosis of a disease, either alone or in combination with another composition, and may have a sterile access port (e.g., the container may be an intravenous solution bag or vial with a stopper pierceable by a hypodermic injection needle). At least one active agent in the composition is the anti-ILT4 antibody of the present disclosure. The label or package insert indicates that the composition is used to treat the selected condition. In addition, the product may comprise: (a) a first container containing a composition, wherein the composition comprises the anti-ILT4 antibody of the present disclosure; and (b) a second container containing a composition, wherein the composition comprises an additional cytotoxic agent or a therapeutic agent of other aspects. The product in this embodiment of the present disclosure may further comprise a package insert indicating that the composition may be used to treat a particular condition. Alternatively or additionally, the product may further comprise a second (or third) container containing a pharmaceutically acceptable buffer. From a commercial and user standpoint, it may further contain other materials as required, including other buffers, diluents, filters, needles, and syringes.

### EXAMPLES

The present disclosure is further described below with reference to the following examples and test examples, which, however, do not limit the present disclosure. The experimental methods in the examples and test examples of the present disclosure in which specific conditions are not specified are generally performed under conventional conditions such as Antibodies: A Laboratory Manual and Molecular Cloning: A Laboratory Manual by Cold Spring Harbor Laboratory, or under conditions recommended by the manufacturers of the starting materials or commercial products. Reagents without specific origins indicated were commercially available conventional reagents.

### Example 1: Preparation of ILT4 Antigens

With UniProt LIRB2_HUMAN (human LILRB2 protein, Uniprot number: Q8N423) as a template of ILT4, the amino acid sequences of the antigens and assay proteins used in the present disclosure were designed; optionally, ILT4 protein was fused with different tags such as a His tag or an Fc. After cloning into a pTT5 vector (Biovector, CAT #102762), 293 cells were transiently transfected with the vector. After expression and purification, immunization and assay antigens of the present disclosure were obtained.

1. Human-IgG1-Fc-tagged ILT4 protein extracellular domain: ILT4-Fc, as an
immunization antigen.
(SEQ ID NO: 1)
QTGTIPKPTLWAEPDSVITQGSPVTLSCQGSLEAQEYRLYREKKSASWITRIRPELVKNG
QFHIPSITWEHTGRYGCQYYSRARWSELSDPLVLVMTGAYPKPTLSAQPSPVVTSGGRV
TLQCESQVAFGGFILCKEGEEEHPQCLNSQPHARGSSRAIFSVGPVSPNRRWSHRCYG
YDLNSPYVWSSPSDLLELLVPGVSKKPSLSVQPGPVVAPGESLTLQCVSDVGYDRFVLY
KEGERDLRQLPGRQPQAGLSQANFTLGPVSRSYGGQYRCYGAHNLSSECSAPSDPLDI
LITGQIRGTPFISVQPGPTVASGENVTLLCQSWRQFHTFLLTKAGAADAPLRLRSIHEYP
KYQAEFPMSPVTSAHAGTYRCYGSLNSDPYLLSHPSEPLELVVSGPSMGSSPPPTGPIS
TPAGPEDQPLTPTGSDPQSGLGRHLGVEPKSSDKTHTCPPCPAPELLGGPSVFLFPP
KPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
2. His-tagged ILT4 protein extracellular domain: ILT4-His, as an assay antigen.
(SEQ ID NO: 2)
QTGTIPKPTLWAEPDSVITQGSPVTLSCQGSLEAQEYRLYREKKSASWITRIRPELVKNG
QFHIPSITWEHTGRYGCQYYSRARWSELSDPLVLVMTGAYPKPTLSAQPSPVVTSGGRV
TLQCESQVAFGGFILCKEGEEEHPQCLNSQPHARGSSRAIFSVGPVSPNRRWSHRCYG
YDLNSPYVWSSPSDLLELLVPGVSKKPSLSVQPGPVVAPGESLTLQCVSDVGYDRFVLY
KEGERDLRQLPGRQPQAGLSQANFTLGPVSRSYGGQYRCYGAHNLSSECSAPSDPLDI
LITGQIRGTPFISVQPGPTVASGENVTLLCQSWRQFHTFLLTKAGAADAPLRLRSIHEYP
KYQAEFPMSPVTSAHAGTYRCYGSLNSDPYLLSHPSEPLELVVSGPSMGSSPPPTGPIS
TPAGPEDQPLTPTGSDPQSGLGRHLGVHHHHHH
(SEQ ID NO: 3)
MTPIVTVLICLGLSLGPRTHVQTGTIPKPTLWAEPDSVITQGSPVTLSCQGSLEAQEYR
LYREKKSASWITRIRPELVKNGQFHIPSITWEHTGRYGCQYYSRARWSELSDPLVLVMT
GAYPKPTLSAQPSPVVTSGGRVTLQCESQVAFGGFILCKEGEEEHPQCLNSQPHARGS
SRAIFSVGPVSPNRRWSHRCYGYDLNSPYVWSSPSDLLELLVPGVSKKPSLSVQPGPVV
APGESLTLQCVSDVGYDRFVLYKEGERDLRQLPGRQPQAGLSQANFTLGPVSRSYGG
QYRCYGAHNLSSECSAPSDPLDILITGQIRGTPFISVQPGPTVASGENVTLLCQSWRQF
HTFLLTKAGAADAPLRLRSIHEYPKYQAEFPMSPVTSAHAGTYRCYGSLNSDPYLLSHP

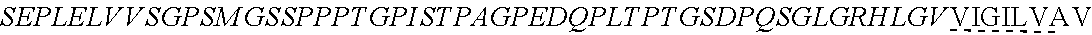

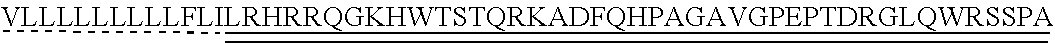

ADAQEENLYAAVKDTQPEDGVEMDTRAAASEAPQDVTYAQLHSLTLRRKATEPP
PSQEREPPAEPSIYATLAIH
Note:
The extracellular domain is italicized, and the human-IgG1-Fc is single underlined.
Note:
The extracellular domain is italicized, and the His tag is single underlined. CHO-S cells (CHO-S/ILT4) overexpressing full-length ILT4 protein were used as assay cells.
Note:
The signal peptide is single underlined, the extracellular domain is italicized, the transmembrane domain is dotted underlined, and the intracellular domain is double underlined.

### Example 2: Purification of ILT4-Associated Recombinant Proteins

### 1. Purification of His-Tagged Recombinant Protein

The cell expression supernatant sample was centrifuged at high speed to remove impurities, the buffer was exchanged for PBS, and imidazole was added until the final concentration was 5 mM. A nickel column was equilibrated with a PBS solution containing 5 mM imidazole and washed with 2-5 column volumes. The cell supernatant sample after exchange was loaded on the Ni Sepharose excel column (GE, 17-3712-02). The column was washed with a PBS solution containing 5 mM imidazole until the A₂₈₀ reading dropped to the baseline. The chromatography column was then washed with PBS+10 mM imidazole to remove non-specifically bound protein impurities, and the eluate was collected. The target protein was eluted with a PBS solution containing 300 mM imidazole, and the eluted peak was collected. The collected eluate was concentrated and further purified using a gel chromatography column Superdex200 (GE, 28-9893-35) with PBS as the mobile phase. The polymer peaks were removed, and the eluted peak was collected. The obtained protein was verified by electrophoresis, peptide mapping, and LC-MS and then aliquoted for later use. ILT4-His, which contains a His tag, was obtained for use as an assay antigen for the antibodies of the present disclosure.

### 2. Purification of Fc-Tagged Recombinant Proteins or Antibodies

The cell expression supernatant sample was centrifuged at high speed to remove impurities, and the supernatant was purified by MabSelect Sure (GE, 17-5438-01) affinity chromatography. The MabSelect Sure chromatography column was regenerated first with 0.2 M NaOH, then washed with purified water, and then equilibrated with PBS. After the supernatant was bound, the column was washed with PBS until the A₂₈₀ reading dropped to the baseline. The target protein was eluted with 0.1 M acetic acid buffer at pH 3.5 and neutralized with 1 M Tris-HCl. The eluted sample was appropriately concentrated and further purified using a gel chromatography column Superdex200 (GE, 28-9893-35) equilibrated with PBS, and the target protein was concentrated to an appropriate concentration in the receiver tube where it was collected. This method was used to purify ILT4-Fc fusion proteins, and it can also be used to purify the related humanized antibody proteins in the present disclosure.

### Example 3: Obtaining of Anti-Human ILT4 Antibodies

### 1. Mouse Immunization

Anti-human ILT4 monoclonal antibodies were produced by immunization of mice. Laboratory SJL white mice, female, aged 6-8 weeks (Shanghai SLAC Laboratory Animal Co., Ltd., animal production license number: SCXK (Shanghai) 2017-0005). Housing environment: SPF. The purchased mice were housed in a laboratory environment for 1 week, with a 12/12-hour light/dark cycle, at a temperature of 20-25° C. with humidity at 40-60%. The acclimatized mice were immunized according to the following scheme.

The mice were immunized with the ILT4-Fc protein. Cross-immunization was performed using the adjuvants TiterMax® Gold Adjuvant (Sigma Cat No. T2684) and Thermo Imject® Alum (Thermo Cat No. 77161). The ratio of the antigen to the adjuvant TiterMax® Gold Adjuvant was 1:1, and the ratio of the antigen to the adjuvant Thermo Imject® Alum was 3:1. The dose for the first immunization was 50 μg/animal/immunization, and the dose for boost immunization was 25 μg/animal/immunization. The antigen was emulsified before inoculation. Immunization time points were 0, 14, and 34 days. Blood samples were collected on days 25 and 41, and the antibody titer in the mouse serum was determined. After the third immunization, mice in which the serum antibody titer was high and tended to plateau were selected for single B-cell screening. Boost immunization was performed 3 days before screening. Each animal was intraperitoneally (i.p.) injected with 25 μg of an antigen solution prepared in normal saline.

### 2. Single B-Cell Screening

Single B-cell screening primarily involved using forward and reverse screening, 10*genomics, and bioinformatics technologies to obtain a series of antibody variable region sequences. The obtained sequences were categorized based on their CDRs. Their murine variable region sequences were selected and linked with human antibody constant region sequences, and chimeric antibodies were expressed. These antibodies were screened through methods such as binding to ILT4 protein, cell binding, and ILT4/HLA-G blocking to obtain antibody sequences that specifically bind to ILT4. The heavy and light chain variable region sequences of the obtained antibodies are shown below:

> M P1-B4 HCVR
(SEQ ID NO: 4)
QVQLKQSGPGLVQPSQSLSITCTVSSFSLTNYGVHWVRQSPGKGLEWLGVIWTG
GSTDYNAAFISRLSIIRDNSKSQVFFKMNSLQADDTAMYYCAITTVVTAMDYWG
QGTSVTVSS
> MP1-B4 LCVR
(SEQ ID NO: 5)
DIVMTQSHKFMSTSVGDRVSITCKASQDVNIAVVWYQQKPGQSPKLLIYAASYRY
TGVPDRFTGSGSGTDFTFTISSVQAEDLAVYYCQQHYSSPYTFGGGTKLEIK
> M P1-B9 HCVR
(SEQ ID NO: 6)
QVQLKQSGPGLVQPSQSLSITCTVSGFSLTSYGVHWVRQSPGKGLEWLGVIWSG
GNTDYNAAFMSRLRINKDNSKSQVFFKMNSLQGSDTAIYYCATPYDVVPFAYWG
QGTLVTVSS
> M P1-B9 LCVR
(SEQ ID NO: 7)
DIVMTQSHKFMSTSVGDRVSITCKASQDVNIAVAWYQQKPGLSPKLLIYSASYRY
TGVPGRFTGSGSGTDFTFTISSVKAEDLAVYYCQQHYSIPFTFGAGTKLEIK
> M P1-D10 HCVR
(SEQ ID NO: 8)
QVQLKQSGPGLVQPSQSLSITCTVSGFSLTTYGVHWVRQSPGKGLEWLGVIWSG
GNTDSNAAFISRLSISKDNSKSQVFFKMNNLQADDTAIYYCARYSYAMDYWGQG
TSVTVSS
> M P1-D10 LCVR
(SEQ ID NO: 9)
DIVMTQSHKFMSTSVGDRVSITCKASQDVSNGVAWYQQKPGQSPKVLIYAASYR
YTGVPDRFTGSGSGTDFTFTIGSVQAEDLALYYCQQHYSTPFTFGSGTMLEIK
> M P2-E8 HCVR
(SEQ ID NO: 10)
EVQLLESGGGLVKPGGSLKLSCAASGFTFSDYGIHWVRQAPEKGLEWVAYISSGS
SIINYADTVKGRFTISRDNAKNTLLLQMTSLRSEDTAMYYCARGPNYYGSSYLLV
YWGQGTLVTVSS
> M P2-E8 LCVR
(SEQ ID NO: 11)
DIVMTQSHKFMSTSVGDRVSITCKASQDVNIAVAWYQQRPGQSPKLLIYSASYRY
TGVPDRFTGSGSGTDFTFTISSVQAEDLAVYYCQQHYNIPWTFGGGTKLEIK
> M P2-C10 HCVR
(SEQ ID NO: 12)
QVQLQQPGAELVKPGASVKLSCKASGYTFTSYWMHWVKQRPGQGLEWIGVIHP
NSDTTNYNEKFKNKATLTVDKSSSTAYMQLSSLTSEDSAVYYCATRYDYAYYAM
DYWGQGTSVTVSS
> M P2-C10 LCVR
(SEQ ID NO: 13)
DIVMTQSHKFMSTSVGDRVSITCKASQDVTTAVAWYQQKPGQSPKLLIYSASYRY
TGVPDRFTGSGSGTDFTFTISSVQAEDLAVYHCQQHYSTSPTFGGGTMLEIK
> M P2-F4 HCVR
(SEQ ID NO: 14)
EVQLVESGGGLVKPGGSLKLSCAASGFTFSDYGMHWVRQAPEKGLEWVAYISSG
SRIIYYADTVKGRFTISRDNAKNTLFLQMTSLRSEDTAMYSCARFYNYAHYTMDY
WGQGTSVTVSS
> M P2-F4 LCVR
(SEQ ID NO: 15)
DIVMTQSHKFMSTSVGDRVSITCKASQDVSTAVAWYQQKPGQSPKLLIYSASYRY
TGVPDRFTGSGSGTDFTFTISSVQAEDLTLYYCQHHYSTPPTFGGGTKLEIK
> M P2-B3 HCVR
(SEQ ID NO: 16)
QVQLKQSGPGLVQPSQSLSITCTVSGFSLINYGVHWFRQSPGKGLEWLGMIWSG
GNTDYNAAFTSRLIITKDNSKSQVFFKMNSMQADDTAIYYCARGDYGSSSAYWG
QGTLVTVSS
> M P2-B3 LCVR
(SEQ ID NO: 17)
DIVMTQSHKFMSTSVGDRVSITCKASQDVSIAVAWYQQKPGQSPKLLIYSASYRY
TGVPDRFTGSGSGTDFTFTISSVQAEDLAVYYCQQHYSNTLTFGAGTKLEIK
> M P2-E7 HCVR
(SEQ ID NO: 18)
QVQLQQPGTELVKPGASVKLSCKASGYTFTSYWMHWVKQSPGQGLEWIGNINP
SNGGPNYNENFKTKATLTVDKSSSTAYMQLNSLTSEDSAVYYCAGGDYYGGSYL
FAYWGQGTLVTVSS
> M P2-E7 LCVR
(SEQ ID NO: 19)
DTVMTQSHKFMSTSVGDRVSVTCKASQDVNIAVAWYQQKPGQSPKLLIYSSSYR
YTGVPDRFTGSGSETDFTFTISSVQAEDLAVYYCQQHYTFPYTFGGGTKLEIK
> M P2-G4 HCVR
(SEQ ID NO: 20)
EVQLQQSGPELVKPGASVKMSCKASGYTFTDYNMHWVKQSHGKSLEWIGYINP
NNGGTTYNQKFKGKATLTVNKSSSTAYMELRSLTSEDSAVYYCASSEYYGSTELE
AYWGQGTLVTVSS
> M P2-G4 LCVR
(SEQ ID NO: 21)
DIVMTQSHKFMSTSVGDRVGITCKASQDVSIAVAWYQQKPGQSPKLLIYSASYRY
TGVPDRFTGSGSGTDFTFTISSVQAEDLAAYYCQQHYSTPWTFGGGTKLEIK
> M P2-C4 HCVR
(SEQ ID NO: 22)
QVQLKESGPGLVAPSQSLSITCTVSGFSLSTYAIGWVRQPPGKGLEWLGVIWTGG
GTSYNSALKSRLSISKDNSKSQVFLKMNSLQTDDTARYYCARGDYGPFAYWGQG
TLVTVSS
> M P2-C4 LCVR
(SEQ ID NO: 23)
DIVMTQSHKFMSTSLGDRVSISCKASQDVSIAVAWYQQKAGQSPKLLIYSASYRH
TGVPDRFTGSGSGTDFTFTISSVQAEDLAVYYCQQHYSTPFTFGSGTKLEIK
>M P2-E11 HCVR
(SEQ ID NO: 24)
QVQLQQSGPELVKPGASVKISCKASAYTFTDYYINWVKQRPGQGLEWIGWIFPGS
GSTYYNEKFKGKATLTVDKSSSTAYMLVSSLTSEDSAVYFCARSGTLVAPYAMDY
WGQGTSVTVSS
> MP2-E11 LCVR
(SEQ ID NO: 25)
SIVMTQTPKFLLVSAGDRVTITCKASQSVSNDVAWYQQKPGQSPKLLIYYTSNRY
TGVPDRFTGSGYGTDFTFTISTVQAEDLAVYFCQQDYSFPLTFGAGTKLEIK
> M P1-F7 HCVR
(SEQ ID NO: 26)
DVQLQESGPGLVKPSQSLSLTCSVTGYSITSGYYWNWIRQFPGNKLEWMGYISYD
GNNYYNPSLKNRISVTRDTSKNQFFLKLNSVTSEDTATYYCAREDGSYSDYDGFA
YWGQGTLVTVSS
> M P1-F7 LCVR
(SEQ ID NO: 27)
DIQMTQSPASLSVSVGETVTITCRANENIYSNLAWYQQKQGKSPQLLVYAATNLA
DGVSSRFSGSGSGTQYSLRINSLQSEDFGSYYCQQFWGTPYTFGGGTKLEIK
> M P2-E3 HCVR
(SEQ ID NO: 28)
EVQLQQSGPELVKPGASVKISCKASGYTFTDYYMNWVKQSHGKSLEWIGDINPN
NGGTSYNQKFKGKATLTVDKSSSTAYMELRSLTSEDSTVYYCARGGVTTVVATY
WYFDVWGTGTTVTVSS
> M P2-E3 LCVR
(SEQ ID NO: 29)
DIQMTQSPASLSVSVGETVTITCRASENIYSNLAWYQQKQGKSPQLLVYAATNVA
DGVPSRFSGGGSGTQYSLKINSLQSEDFGSYYCQQFWDTPFTFGSGTKLEIK
> M P2-F10 HCVR
(SEQ ID NO: 30)
DVQLQESGPGLVKPSQSLSLTCSVTGYSIASGYYWNWIRQFPGNKLEWMGYITY
DGSNNYNPSLKNRISITRDTSKNQFFLKLNSVTTEDTATYYCAREYSYGSTLSWFA
YWGQGTLVTVSS
> M P2-F10 LCVR
(SEQ ID NO: 31)
DIQMTQSPASLSISVGETVTITCRASENIYSNLAWYQQKQGKSPQLLVYAATNLAD
GVPSRFSGSGSGTQYSLKINSLQSEDFGSYYCQHFWDTPYTFGGGTKLEIK
> M P2-G9 HCVR
(SEQ ID NO: 32)
DVQLQESGPGLVKPSQSLSLACSVTGSSITSGYYWNWIRQFPGNKLEWMGYLSY
DGSNNYNPSLKNRISITRDTSKSQFFLKLNSVTPEDTATYYCAREGAHYSGTLSWF
AYWGQGTLVTVSS
> M P2-G9 LCVR
(SEQ ID NO: 33)
DIQMTQSPASLSVSVGETVTITCRASENIYSNLAWYQQKQGKSPQLLVYAATNLA
DGVPSRFSGSGSGTQYSLKINSLQSEDFGSYYCQHFWDTPYTFGGGTKLEIK
> M P1-C4 HCVR
(SEQ ID NO: 34)
QVQLKQSGAELVRPGASVKLSCKASGYSFTDYYINWVKQRPGQGLEWIARIYPG
NDNTYYNEKFKDKATLTADSSSSTAYMQLSSLTSEDSAVYFCTRSQDGYYEENAM
DYWGQGTSVTVSS
> M P1-C4 LCVR
(SEQ ID NO: 35)
DIQMTQSPASLSVSVGETVTITCRASENIYSNLAWYQQRQGKSPQLLVYAATNLA
DGVPFRFSGSGSGTQYSLKINSLQSEDFGSYYCQQFWGSPHTFGGGTKLEIK
> M P1-G7 HCVR
(SEQ ID NO: 36)
DVQLQESGPGLVKPSQSLSLTCSVTGYSITSGYYWNWIRQFPENKLEWMGYISYD
GGNNYNPFLKNRISITRDTSKNQFFLNLNSVTIEDTATYYCAREGGSSWEYYGMD
YWGQGTSVTVSS
> M P1-G7 LCVR
(SEQ ID NO: 37)
DIQMTQSPASLSVSVGETVTITCRASENIYRNLAWYQQKQGHSPQLLLYAATNLA
DGVPSRFSGSGSGTQYSLKINSLQSEDFGSYYCQQFWGSPYTFGGGTKLEIK
> M P2-C7 HCVR
(SEQ ID NO: 38)
EVQLQQSGPELVKPGTSVKMSCKASGYTFTDYSMHWVRQSHGKSLEWIGYFNP
NINGDTIYNQKFKGKATLTVNKSSSTAYMELRSLTSDDSAVYYCAREDSVSTNAW
DGALDYWGQGTSVTVSS
> M P2-C7 LCVR
(SEQ ID NO: 39)
DIQMTQSPASLSVSVGETVTITCRTSENIYSNLAWYQQKQGKSPQLLVYGATNLA
DGVPSRFSGSGSGTQYSLKINSLQSEDFGSYYCQHFWDIPWTFGGGTTLEIK
> M P2-C6 HCVR
(SEQ ID NO: 40)
EVQLQQSGPELVKPGASVKMSCEASGYTFTDYYIHWVKQSHGESLEWIGYIYPD
NGYNGYNQKFKGKATLTVDKSSNTAYMELRSLTSEDSAVYFCTRRGDYSGVHED
YWGQGTALTVSS
> M P2-C6 LCVR
(SEQ ID NO: 41)
DIVLTQSPASLAVSLGQRATISCRASESVNNYGVGFLHWYQQKPGQPPKLLIYRAS
TLESGIPARFSGSGSRTDFTLTINPVETDDVATYYCQHSYKDPPWTFGGGTKLEIK
> M P1-D7 HCVR
(SEQ ID NO: 42)
QIQLVQSGPDLKKPGETVKISCKASGYTFTTYGMNWVKQAPGKALKWMGWINT
YSGVPTYADDFKGRFAFSLETSATTAYLQINNLKNEDTATYFCARRTDYDGYVMD
CWGQGTSVTVSS
> M P1-D7 LCVR
(SEQ ID NO: 43)
DIVMSQSPSSLAVSIGEKVTMSCKSSQSLFYSGNQKNYLAWYLQKPGQSPKLLLY
WASTRESGVPDRFTGSGSGTDFTLTISSVMAEDLAVYYCQQYFSYPPTFGGGTKL
EIK
> MP1-E11 HCVR
(SEQ ID NO: 44)
EVQLQQSGPELVKPGASVKMSCQASGYTFTDYSIHWVKQSRGASLEWIGYINPN
NGVTNYNQKFKGKATLTVNKSSSTAYMELRSLTSEDSAVYYCAREGDSVTTIVVH
WYFDVGGTGTTVTVSS
> MP1-E11 LCVR
(SEQ ID NO: 45)
DIQMTQSPASLSVSLGETVTITCRASENIYSNLVWYQQKQGKFPQLLVYAAINLVD
GVPSRFSGSGSGTQYSLKINSLQSEDFGSYYCQQFWDTPLTFGAGTKLEIK
> M P2-B9 HCVR
(SEQ ID NO: 46)
EVQLQQSGPELVKPGASVKISCKASGYTFTDYFMNWVKLSHGKSLEWIGDINPN
NGGTSYNQRFKDKVTLTVDKSSSTAYMELRSLTSDSSAVYFCARNGYYDSNYLS
YFDVWGTGTTVTVSS
> M P2-B9 LCVR
(SEQ ID NO: 47)
DIQMTQSPASLSVSVGETVTITCRASENIYSNLAWYQQKQGKSPQLLVYGATNLA
DGVPSRFSGSGSGTQYSLKINSLQSEDFGSYYCQHEWDTPYTFGGGTKLEIK
> M P2-C2 HCVR
(SEQ ID NO: 48)
QVQLKQSGPGLVQPSQSLSITCTVSGFSLTSYGVHWVRQSPGKGLEWLGVIWSG
GHTDLNAAFISRLSINKDNSKSQVFFKMNSLQGDDTAIYYCARNGYGGCLDYWG
QGTTLTVSS
> M P2-C2 LCVR
(SEQ ID NO: 49)
DIVMTQSHKFMSTSVGDRVSITCKAGQDVSIAVAWYQQKPGQSPQLLIYSASYRY
TGVPDRFTGSGSGTDFTFAISSVQAEDLAVYYCQQYYSIPPSFGAGTKLEIK
>M P2-E10 HCVR
(SEQ ID NO: 50)
DVQLQESGPGLVKPSQSLSLTCSVTGYSITSGYYWNWIRQFPGNKLEWMGYMSY
EGTNNSNPSLKNRISITRDTSKNQFFLKLNSVTTEDTARYYCARERAYYYGTLGA
MDYWGQGTSVTVSS
> M P2-E10 LCVR
(SEQ ID NO: 51)
DIQMTQSPASLSVSVGETVTITCRASENIYSNLAWYQQKQGKSPQLLVYAATNLA
DGVPSRFSGSGSGTQYSLKINSMQSEDFGSYYCQHFWDTPFTFGSGTKLEIK
Note: The variable regions are single underlined,
and the CDRs are double underlined;
the CDRs are defined according to the Kabat
numbering scheme.

The heavy and light chain CDR sequences of the murine antibodies are shown in Table 3 below.

TABLE 3
The heavy and light chain CDR sequences of the antibodies

| Antibody | Heavy chain | | Light chain | |
|---|---|---|---|---|
| MP1-B4 | HCDR1 | NYGVH | LCDR1 | KASQDVNIAVV (SEQ ID |
| | | (SEQ ID NO: 52) | | NO: 55) |
| | HCDR2 | VIWTGGSTDYNAAFIS | LCDR2 | AASYRYT |
| | | (SEQ ID NO: 53) | | (SEQ ID NO: 56) |
| | HCDR3 | TTVVTAMDY (SEQ ID NO: | LCDR3 | QQHYSSPYT (SEQ ID NO: |
| | | 54) | | 57) |
| MP1-B9 | HCDR1 | SYGVH | LCDR1 | KASQDVNIAVA (SEQ ID |
| | | (SEQ ID NO: 58) | | NO: 61) |
| | HCDR2 | VIWSGGNTDYNAAFMS | LCDR2 | SASYRYT |
| | | (SEQ ID NO: 59) | | (SEQ ID NO: 62) |
| | HCDR3 | PYDVVPFAY (SEQ ID NO: | LCDR3 | QQHYSIPFT (SEQ ID NO: |
| | | 60) | | 63) |
| MP1-D10 | HCDR1 | TYGVH | LCDR1 | KASQDVSNGVA (SEQ ID |
| | | (SEQ ID NO: 64) | | NO: 67) |
| | HCDR2 | VIWSGGNTDSNAAFIS | LCDR2 | AASYRYT |
| | | (SEQ ID NO: 65) | | (SEQ ID NO: 56) |
| | HCDR3 | YSYAMDY | LCDR3 | QQHYSTPFT (SEQ ID NO: |
| | | (SEQ ID NO: 66) | | 68) |
| MP2-E8 | HCDR1 | DYGIH | LCDR1 | KASQDVNIAVA (SEQ ID |
| | | (SEQ ID NO: 69) | | NO:61) |
| | HCDR2 | YISSGSSIINYADTVKG | LCDR2 | SASYRYT |
| | | (SEQ ID NO: 70) | | (SEQ ID NO: 62) |
| | HCDR3 | GPNYYGSSYLLVY (SEQ ID | LCDR3 | QQHYNIPWT (SEQ ID NO: |
| | | NO: 71) | | 72) |
| MP2-C10 | HCDR1 | SYWMH | LCDR1 | KASQDVTTAVA (SEQ ID |
| | | (SEQ ID NO: 73) | | NO: 76) |
| | HCDR2 | VIHPNSDTTNYNEKFKN | LCDR2 | SASYRYT |
| | | (SEQ ID NO: 74) | | (SEQ ID NO: 62) |
| | HCDR3 | RYDYAYYAMDY (SEQ ID | LCDR3 | QQHYSTSPT (SEQ ID NO: |
| | | NO: 75) | | 77) |
| MP2-F4 | HCDR1 | DYGMH | LCDR1 | KASQDVSTAVA (SEQ ID |
| | | (SEQ ID NO: 78) | | NO: 81) |
| | HCDR2 | YISSGSRIIYYADTVKG | LCDR2 | SASYRYT |
| | | (SEQ ID NO: 79) | | (SEQ ID NO: 62) |
| | HCDR3 | FYNYAHYTMDY (SEQ ID | LCDR3 | QHHYSTPPT (SEQ ID NO: |
| | | NO: 80) | | 82) |
| MP2-B3 | HCDR1 | NYGVH | LCDR1 | KASQDVSIAVA (SEQ ID |
| | | (SEQ ID NO: 52) | | NO: 85) |
| | HCDR2 | MIWSGGNTDYNAAFTS | LCDR2 | SASYRYT |
| | | (SEQ ID NO: 83) | | (SEQ ID NO: 62) |
| | HCDR3 | GDYGSSSAY (SEQ ID NO: | LCDR3 | QQHYSNTLT (SEQ ID NO: |
| | | 84) | | 86) |
| MP2-E7 | HCDR1 | SYWMH | LCDR1 | KASQDVNIAVA (SEQ ID |
| | | (SEQ ID NO: 73) | | NO: 61) |
| | HCDR2 | NINPSNGGPNYNENFKT | LCDR2 | SSSYRYT |
| | | (SEQ ID NO: 87) | | (SEQ ID NO: 89) |
| | HCDR3 | GDYYGGSYLFAY (SEQ ID | LCDR3 | QQHYTFPYT (SEQ ID NO: |
| | | NO: 88) | | 90) |
| MP2-G4 | HCDR1 | DYNMH | LCDR1 | KASQDVSIAVA (SEQ ID |
| | | (SEQ ID NO: 91) | | NO: 85) |
| | HCDR2 | YINPNNGGTTYNQKFKG | LCDR2 | SASYRYT |
| | | (SEQ ID NO: 92) | | (SEQ ID NO: 62) |
| | HCDR3 | SEYYGSTFLFAY (SEQ ID | LCDR3 | QQHYSTPWT (SEQ ID NO: |
| | | NO: 93) | | 94) |
| MP2-C4 | HCDR1 | TYAIG | LCDR1 | KASQDVSIAVA (SEQ ID |
| | | (SEQ ID NO: 95) | | NO: 85) |
| | HCDR2 | VIWTGGGTSYNSALKS | LCDR2 | SASYRHT |
| | | (SEQ ID NO: 96) | | (SEQ ID NO: 98) |
| | HCDR3 | GDYGPFAY (SEQ ID NO: | LCDR3 | QQHYSTPFT (SEQ ID |
| | | 97) | | NO:68) |
| MP2-E11 | HCDR1 | DYYIN | LCDR1 | KASQSVSNDVA (SEQ ID |
| | | (SEQ ID NO: 99) | | NO: 102) |
| | HCDR2 | WIFPGSGSTYYNEKFKG | LCDR2 | YTSNRYT (SEQ ID NO: |
| | | (SEQ ID NO: 100) | | 103) |
| | HCDR3 | SGTLVAPYAMDY (SEQ ID | LCDR3 | QQDYSFPLT (SEQ ID NO: |
| | | NO: 101) | | 104) |
| MP1-F7 | HCDR1 | SGYYWN | LCDR1 | RANENIYSNLA (SEQ ID |
| | | (SEQ ID NO: 105) | | NO: 108) |
| | HCDR2 | YISYDGNNYYNPSLKN | LCDR2 | AATNLAD (SEQ ID NO: |
| | | (SEQ ID NO: 106) | | 109) |
| | HCDR3 | EDGSYSDYDGFAY | LCDR3 | QQFWGTPYT (SEQ ID NO: |
| | | (SEQ ID NO: 107) | | 110) |
| MP2-E3 | HCDR1 | DYYMN | LCDR1 | RASENIYSNLA (SEQ ID |
| | | (SEQ ID NO: 111) | | NO: 114) |
| | HCDR2 | DINPNNGGTSYNQKFKG | LCDR2 | AATNVAD (SEQ ID NO: |
| | | (SEQ ID NO: 112) | | 115) |
| | HCDR3 | GGVTTVVATYWYFDV | LCDR3 | QQFWDTPFT (SEQ ID NO: |
| | | (SEQ ID NO: 113) | | 116) |
| MP2-F10 | HCDR1 | SGYYWN | LCDR1 | RASENIYSNLA (SEQ ID |
| | | (SEQ ID NO: 105) | | NO: 114) |
| | HCDR2 | YITYDGSNNYNPSLKN | LCDR2 | AATNLAD (SEQ ID NO: |
| | | (SEQ ID NO: 117) | | 109) |
| | HCDR3 | EYSYGSTLSWFAY (SEQ ID | LCDR3 | QHFWDTPYT (SEQ ID NO: |
| | | NO: 118) | | 119) |
| MP2-G9 | HCDR1 | SGYYWN | LCDR1 | RASENIYSNLA (SEQ ID |
| | | (SEQ ID NO: 105) | | NO: 114) |
| | HCDR2 | YLSYDGSNNYNPSLKN | LCDR2 | AATNLAD (SEQ ID NO: |
| | | (SEQ ID NO: 120) | | 109) |
| | HCDR3 | EGAHYSGTLSWFAY (SEQ | LCDR3 | QHFWDTPYT (SEQ ID NO: |
| | | ID NO: 121) | | 119) |
| MP1-C4 | HCDR1 | DYYIN | LCDR1 | RASENIYSNLA (SEQ ID |
| | | (SEQ ID NO: 99) | | NO: 114) |
| | HCDR2 | RIYPGNDNTYYNEKFKD | LCDR2 | AATNLAD (SEQ ID NO: |
| | | (SEQ ID NO: 122) | | 109) |
| | HCDR3 | SQDGYYEENAMDY (SEQ | LCDR3 | QQFWGSPHT (SEQ ID NO: |
| | | ID NO: 123) | | 124) |
| MP1-G7 | HCDR1 | SGYYWN | LCDR1 | RASENIYRNLA (SEQ ID |
| | | (SEQ ID NO: 105) | | NO: 127) |
| | HCDR2 | YISYDGGNNYNPFLKN | LCDR2 | AATNLAD (SEQ ID NO: |
| | | (SEQ ID NO: 125) | | 109) |
| | HCDR3 | EGGSSWEYYGMDY (SEQ | LCDR3 | QQFWGSPYT (SEQ ID NO: |
| | | ID NO: 126) | | 128) |
| MP2-C7 | HCDR1 | DYSMH | LCDR1 | RTSENIYSNLA (SEQ ID |
| | | (SEQ ID NO: 129) | | NO: 132) |
| | HCDR2 | YFNPNNGDTIYNQKFKG | LCDR2 | GATNLAD (SEQ ID NO: |
| | | (SEQ ID NO: 130) | | 133) |
| | HCDR3 | EDSVSTNAWDGALDY | LCDR3 | QHFWDIPWT (SEQ ID NO: |
| | | (SEQ ID NO: 131) | | 134) |
| MP2-C6 | HCDR1 | DYYIH | LCDR1 | RASESVNNYGVGFLH |
| | | (SEQ ID NO: 135) | | (SEQ ID NO: 138) |
| | HCDR2 | YIYPDNGYNGYNQKFKG | LCDR2 | RASTLES (SEQ ID NO: |
| | | (SEQ ID NO: 136) | | 139) |
| | HCDR3 | RGDYSGVHFDY (SEQ ID | LCDR3 | QHSYKDPPWT (SEQ ID |
| | | NO: 137) | | NO: 140) |
| MP1-D7 | HCDR1 | TYGMN | LCDR1 | KSSQSLFYSGNQKNYLA |
| | | (SEQ ID NO: 141) | | (SEQ ID NO: 144) |
| | HCDR2 | WINTYSGVPTYADDFKG | LCDR2 | WASTRES |
| | | (SEQ ID NO: 142) | | (SEQ ID NO: 145) |
| | HCDR3 | RTDYDGYVMDC (SEQ ID | LCDR3 | QQYFSYPPT (SEQ ID NO: |
| | | NO: 143) | | 146) |
| MP1-E11 | HCDR1 | DYSIH | LCDR1 | RASENIYSNLV (SEQ ID |
| | | (SEQ ID NO: 147) | | NO: 150) |
| | HCDR2 | YINPNNGVTNYNQKFKG | LCDR2 | AAINLVD (SEQ ID NO: |
| | | (SEQ ID NO: 148) | | 151) |
| | HCDR3 | EGDSVTTIVVHWYFDV | LCDR3 | QQFWDTPLT (SEQ ID NO: |
| | | (SEQ ID NO: 149) | | 152) |
| MP2-B9 | HCDR1 | DYFMN | LCDR1 | RASENIYSNLA (SEQ ID |
| | | (SEQ ID NO: 153) | | NO: 114) |
| | HCDR2 | DINPNNGGTSYNQRFKD | LCDR2 | GATNLAD (SEQ ID NO: |
| | | (SEQ ID NO: 154) | | 133) |
| | HCDR3 | NGYYDSNYLSYFDV (SEQ | LCDR3 | QHFWDTPYT (SEQ ID NO: |
| | | ID NO: 155) | | 119) |
| MP2-C2 | HCDR1 | SYGVH | LCDR1 | KAGQDVSIAVA (SEQ ID |
| | | (SEQ ID NO: 58) | | NO: 158) |
| | HCDR2 | VIWSGGHTDLNAAFIS | LCDR2 | SASYRYT |
| | | (SEQ ID NO: 156) | | (SEQ ID NO: 62) |
| | HCDR3 | NGYGGCLDY | LCDR3 | QQYYSIPPS |
| | | (SEQ ID NO: 157) | | (SEQ ID NO: 159) |
| MP2-E10 | HCDR1 | SGYYWN | LCDR1 | RASENIYSNLA (SEQ ID |
| | | (SEQ ID NO: 105) | | NO: 114) |
| | HCDR2 | YMSYEGTNNSNPSLKN | LCDR2 | AATNLAD (SEQ ID NO: |
| | | (SEQ ID NO: 160) | | 109) |
| | HCDR3 | ERAYYYGTLGAMDY (SEQ | LCDR3 | QHFWDTPFT (SEQ ID NO: |
| | | ID NO: 161) | | 162) |

### 3. Phage Library Screening

Since the screening was aimed at obtaining antibody sequences that bind only to ILT4, reverse screening and forward screening were combined: phages that bind to biotinylated human ILT2 were removed first from a phage library (reverse screening), and phages that bind to biotinylated human ILT4 were then selected from the remaining phages (forward screening). Specifically, the phage library (5×10¹²/pfu) was suspended in 1 mL of PBS containing skim milk powder (BD, 232100), 2 μg/mL biotinylated human ILT2-His protein was added, and incubation was performed in an inverted position. Subsequently, 100 μL of Dynabeads® M-280 Streptavidin (Thermofisher, 11206D) was added, and incubation was performed. The tube was placed on a magnetic rack for 30 s, and the supernatant was taken out. 2 μg/mL biotinylated human ILT4-His protein was added, and incubation was performed in an inverted position. Subsequently, 100 μL of Dynabeads was added, and incubation was performed. The tube was placed on a magnetic rack for 30 s, and the supernatant was removed. The Dynabeads were washed several times with 1 mL of PBS containing Tween-20, and 0.5 mL of trypsin (Sigma, T1426-250 MG) was then added for elution. The eluted phages were used to infect E. coli SS320. The titer was determined, and expansion and concentration were performed. The resulting phages were used in the next round of panning. After three rounds of screening based on this method, the obtained single clones were inoculated into a 96-well plate (Sangon Biotech, F600582-0001) for phage packaging. Phages that bind to human ILT4 protein and do not bind to human ILT2 protein were identified by ELISA, and the corresponding single clones were sequenced. The variable region sequences of the finally obtained antibody are shown below:

> P2 HCVR
(SEQ ID NO: 163)
QVQLVQSGAEVKKPGSSVKVSCKASGYTFTSYDISWVRQAPGQGLEWMGGIIPIF
GTANYAQKFQGRVTITADESTSTAYMELSSLRSEDTAVYYCTAGIFLATAYWGQGT
LVTVSS
> P2 LCVR
(SEQ ID NO: 164)
DIQMTQSPSSMSASVGDRVTISCQASQDINNYLNWYQQRPGQAPKLLIYDASTLET
GVPSRFSGSGSGTYFTLTITSLQPADVATYYCQQYENVPITFGQGTRLEIK

Note: The variable regions are single underlined, and the CDRs are double underlined; the CDRs are defined according to the Kabat numbering scheme.

The heavy and light chain CDR sequences of the fully human-derived antibody are shown in Table 4 below.

TABLE 4
The heavy and light chain CDR sequences
of the antibody

| Antibody | Heavy chain | | Light chain | |
|---|---|---|---|---|
| P2 | HCDR1 | SYDIS (SEQ ID | LCDR1 | QASQDINNYLN |
| | | NO: 165) | | (SEQ ID NO: 168) |
| | HCDR2 | GIIPIFGTANYAQ | LCDR2 | DASTLET (SEQ ID |
| | | KFQG (SEQ ID | | NO: 169) |
| | | NO: 166) | | |
| | HCDR3 | GIFLATAY (SEQ | LCDR3 | QQYENVPIT (SEQ |
| | | ID NO: 167) | | ID NO: 170) |

### 4. Construction of Chimeric Antibodies

Primers were designed, and VH/VK gene fragments of the murine antibodies were obtained by PCR. The fragments were then homologously recombined with an expression vector pTT5 (with a signal peptide and a constant region gene (CH1-FC/CL) fragment, constructed in the laboratory) to construct an antibody full-length expression vector VH-CH1-FC-pTT5/VK-CL-pTT5, and anti-human ILT4 chimeric antibodies in IgG4 (LALA) format were expressed. The variable region sequences of the murine anti-ILT4 antibodies ChP1-C4, ChP1-B4, ChP1-D10, ChP2-C7, ChP1-G7, ChP1-B9, ChP2-C6, ChP1-F7, ChP2-E8, ChP2-E3, ChP2-C10, ChP2-F4, ChP2-F10, ChP2-C4, ChP2-G9, ChP2-B3, ChP2-E7, ChP2-G4, ChP2-E11, ChP1-D7, ChP1-E11, ChP2-B9, ChP2-C2, and ChP2-E10 were combined with the constant regions set forth in SEQ ID NO: 171 and SEQ ID NO: 172 to obtain chimeric antibodies. Illustratively, ChP1-C4 represents a chimeric antibody containing the M P1-C4 murine heavy and light chain variable regions and the constant regions. The heavy and light chain constant region sequences of the chimeric antibodies are shown below:

> hIgG4(LALA)
(SEQ ID NO: 171)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV

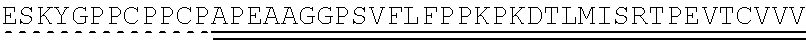

DVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVS
QVSLTCLVKGFYPSDIAVEWESNGQPENNYKTTPPVLDSDGSFFLYSRL
TVDKSRWQEGNVESCSVMHEALHNHYTQKSLSLSLGK
Note:
The CH1 region is single underlined, the
hinge region is dotted underlined, and the
Fc region is double underlined.
> CL
(SEQ ID NO: 172)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

### Example 4: Humanization of Anti-Human ILT4 Murine Antibodies

By comparing the IMGT human antibody heavy and light chain variable region germline gene database and MOE software, heavy and light chain variable region germline genes that are highly homologous with M P2-E3, M P2-G9, M P2-E8, M P1-G7, M P2-C6, and M P1-D7 were selected as templates, and the CDRs of the six murine antibodies were grafted into corresponding humanization templates to form variable region sequences with the order FR1-CDR1-FR2-CDR2-FR3-CDR3-FR4. The amino acid residues were determined using the Kabat numbering scheme and annotated.

### 1. Humanization Engineering of M P2-E3

For humanized antibodies of the M P2-E3 antibody, IGKV1-39*01 was selected as a light chain framework region template, and IGHV1-3*01 was selected as a heavy chain framework region template. The CDRs of the murine antibodies were grafted onto the selected humanization templates, and embedded residues, residues directly interacting with the CDRs, and sites with high-risk hotspots and immunogenicity risks were identified using MOE software and back-mutated and point-mutated to design different light and heavy chain variable regions for humanized antibodies.

TABLE 5
The specific framework replacements and amino acid
substitutions for P2-E3 humanized antibodies

| | |
|---|---|
| VL | VH |

| | | | |
|---|---|---|---|
| L1 | Graft(IGKV1-39*01) + | H1 | Graft(IGHV1-3*01) + Q1E, R71V, |
| | I48V | | T73K |
| L2 | Graft(IGKV1-39*01) + | H2 | Graft(IGHV1-3*01) + Q1E, R44S, |
| | A43S, I48V, F71Y | | I69L, R71V, T73K |
| | | H3 | Graft(IGHV1-3*01) + Q1E, R44S, |
| | | | I69L, R71V, T73K + N54S |
| | | H4 | Graft(IGHV1-3*01) + Q1E, R44S, |
| | | | I69L, R71V, T73K + N54T |
| | | H5 | Graft(IGHV1-3*01) + Q1E, R44S, |
| | | | I69L, R71V, T73K + G55V |
| | | H6 | Graft(IGHV1-3*01) + Q1E, R38K, |
| | | | R71V, T73K, A75G + N54S |
| FR4 | IGKJ2*01 | FR4 | IGHJ6*01 |

Note:
The amino acids are numbered according to the Kabat numbering scheme. I48V indicates that the I at position 48, according to the Kabat numbering scheme, is mutated into V, and so on for other mutations.

TABLE 6
The CDRs of P2-E3 humanized antibodies

| Variable | | | SEQ ID |
|---|---|---|---|
| region | CDR | Sequence | NO |
| huP2-E3 VH3 | HCDR2 | DINPNSGGTSYNQKFKG | 173 |
| huP2-E3 VH4 | HCDR2 | DINPNTGGTSYNQKFKG | 174 |
| huP2-E3 VH5 | HCDR2 | DINPNNVGTSYNQKFKG | 175 |
| huP2-E3 VH6 | HCDR2 | DINPNSGGTSYNQKFKG | 173 |

The variable region sequences of P2-E3 humanized antibodies are shown below:

> huP2-E3 VH1
(SEQ ID NO: 176)
EVQLVQSGAEVKKPGASVKVSCKASGYTFTDYYMNWVRQAPGQRLEWM
GDINPNNGGTSYNQKFKGRVTITVDKSASTAYMELSSLRSEDTAVYYC
ARGGVTTVVATYWYFDVWGQGTTVTVSS
> huP2-E3 VH2
(SEQ ID NO: 177)
EVQLVQSGAEVKKPGASVKVSCKASGYTFTDYYMNWVRQAPGQSLEWMG
DINPNNGGTSYNQKFKGRVTLTVDKSASTAYMELSSLRSEDTAVYYCAR
GGVTTVVATYWYFDVWGQGTTVTVSS
> huP2-E3 VH3
(SEQ ID NO: 178)
EVQLVQSGAEVKKPGASVKVSCKASGYTFTDYYMNWVRQAPGQSLEWMG
DINPNSGGTSYNQKFKGRVTLTVDKSASTAYMELSSLRSEDTAVYYCAR
GGVTTVVATYWYFDVWGQGTTVTVSS
> huP2-E3 VH4
(SEQ ID NO: 179)
EVQLVQSGAEVKKPGASVKVSCKASGYTFTDYYMNWVRQAPGQSLEWMG
DINPNTGGTSYNQKFKGRVTLTVDKSASTAYMELSSLRSEDTAVYYCAR
GGVTTVVATYWYFDVWGQGTTVTVSS
> huP2-E3 VH5
(SEQ ID NO: 180)
EVQLVQSGAEVKKPGASVKVSCKASGYTFTDYYMNWVRQAPGQSLEWMG
DINPNNVGTSYNQKFKGRVTLTVDKSASTAYMELSSLRSEDTAVYYCAR
GGVTTVVATYWYFDVWGQGTTVTVSS
> huP2-E3 VH6
(SEQ ID NO: 181)
EVQLVQSGAEVKKPGASVKVSCKASGYTFTDYYMNWVKQAPGQRLEWMG
DINPNSGGTSYNQKFKGRVTITVDKSGSTAYMELSSLRSEDTAVYYCAR
GGVTTVVATYWYFDVWGQGTTVTVSS
> huP2-E3 VL1
(SEQ ID NO: 182)
DIQMTQSPSSLSASVGDRVTITCRASENIYSNLAWYQQKPGKAPKLLVY
AATNVADGVPSRFSGSGSGTDFTLTISSLQPEDFATYYCQQFWDTPFTF
GQGTKLEIK
> huP2-E3 VL2
(SEQ ID NO: 183)
DIQMTQSPSSLSASVGDRVTITCRASENIYSNLAWYQQKPGKSPKLLVY
AATNVADGVPSRFSGSGSGTDYTLTISSLQPEDFATYYCQQFWDTPFTF
GQGTKLEIK
Note: The variable regions are single underlined, and
the CDRs are double underlined; the
CDRs are defined according to the Kabat numbering scheme.

TABLE 7
Humanized antibodies of the P2-E3 series

| | huP2-E3 | huP2-E3 | huP2-E3 | huP2-E3 | huP2-E3 | |
|---|---|---|---|---|---|---|
| | VH1 | VH2 | VH3 | VH4 | VH5 | huP2-E3 VH6 |
| huP2-E3 | P2- | P2- | P2- | P2- | P2- | P2-E3H6L1 |
| VL1 | E3H1L1 | E3H2L1 | E3H3L1 | E3H4L1 | E3H5L1 | |
| huP2-E3 | P2- | P2- | P2- | P2- | P2- | P2-E3H6L2 |
| VL2 | E3H1L2 | E3H2L2 | E3H3L2 | E3H4L2 | E3H5L2 | (also known as |
| | | | | | | huP2-E3) |

### 2. Humanization Engineering of M P2-G9

For humanized antibodies of the M P2-G9 antibody, IGKV1-39*01 was selected as a light chain framework region template, and IGHV4-31*01 was selected as a heavy chain framework region template. The CDRs of the murine antibodies were grafted onto the selected humanization templates, and embedded residues, residues directly interacting with the CDRs, and sites with high-risk hotspots and immunogenicity risks were identified using MOE software and back-mutated and point-mutated to design different light and heavy chain variable regions for humanized antibodies.

TABLE 8
The specific framework replacements and amino acid
substitutions for P2-G9 humanized antibodies

| VL | | VH | |
|---|---|---|---|
| L1 | Graft(IGKV1-39*01) + | H1 | Graft(IGHV4-31*01) + G27S, |
| | I48V | | S30T, V71R + Q1E |
| L2 | Graft(IGKV1-39*01) + | H2 | Graft(IGHV4-31*01) + G27S, |
| | A43S, I48V, F71Y | | S30T, G44K, V71R, N76S + |
| | | | Q1E |
| FR4 | IGKJ4*01 | FR4 | IGHJ1*01 |

Note:
The amino acids are numbered according to the Kabat numbering scheme.

The variable region sequences of P2-G9 humanized antibodies are shown below:

> huP2-G9 VH1
(SEQ ID NO: 184)
EVQLQESGPGLVKPSQTLSLTCTVSGSSITSGYYWNWIRQHPGKG
LEWIGYLSYDGSNNYNPSLKNLVTISRDTSKNQFSLKLSSVTAAD
TAVYYCAREGAHYSGTLSWFAYWGQGTLVTVSS
> huP2-G9 VH2
(SEQ ID NO: 185)
EVQLQESGPGLVKPSQTLSLTCTVSGSSITSGYYWNWIRQHPGKK
LEWIGYLSYDGSNNYNPSLKNLVTISRDTSKSQFSLKLSSVTAAD
TAVYYCAREGAHYSGTLSWFAYWGQGTLVTVSS
> huP2-G9 VL1
(SEQ ID NO: 186)
DIQMTQSPSSLSASVGDRVTITCRASENIYSNLAWYQQKPGKAPK
LLVYAATNLADGVPSRFSGSGSGTDFTLTISSLQPEDFATYYCQH
FWDTPYTFGGGTKVEIK
> huP2-G9 VL2
(SEQ ID NO: 187)
DIQMTQSPSSLSASVGDRVTITCRASENIYSNLAWYQQKPGKSPK
LLVYAATNLADGVPSRFSGSGSGTDYTLTISSLQPEDFATYYCQH
FWDTPYTFGGGTKVEIK
Note: The variable regions are single underlined,
and the CDRs are double underlined; the CDRs are
defined according to the Kabat numbering scheme.

TABLE 9-1
Humanized antibodies of the P2-G9 series

| | huP2-G9 VH1 | huP2-G9 VH2 |
|---|---|---|
| huP2-G9 VL1 | P2-G9H1L1 | P2-G9H2L1 |
| huP2-G9 VL2 | P2-G9H1L2 | P2-G9H2L2 |
| | (also known as huP2-G9) | |

TABLE 9-2
The CDR sequences of the huP2-G9 antibodies
(according to the Kabat numbering scheme)

| Antibody | Name | Sequence | No. |
|---|---|---|---|
| huP2-G9 | HCDR1 | SGYYWN | SEQ ID NO: 105 |
| | HCDR2 | YLSYDGSNNYNPSLKN | SEQ ID NO: 120 |
| | HCDR3 | EGAHYSGTLSWFAY | SEQ ID NO: 121 |
| | LCDR1 | RASENIYSNLA | SEQ ID NO: 114 |
| | LCDR2 | AATNLAD | SEQ ID NO: 109 |
| | LCDR3 | QHFWDTPYT | SEQ ID NO: 119 |

TABLE 9-3
The CDR sequences of the huP2-G9 antibodies
(according to the IMGT numbering scheme)

| Antibody | Name | Sequence | No. |
|---|---|---|---|
| huP2-G9 | HCDR1 | GSSITSGYY | SEQ ID NO: 260 |
| | HCDR2 | LSYDGSN | SEQ ID NO: 261 |
| | HCDR3 | AREGAHYSGTLSWFAY | SEQ ID NO: 262 |
| | LCDR1 | ENIYSN | SEQ ID NO: 263 |
| | LCDR2 | AAT | SEQ ID NO: 264 |
| | LCDR3 | QHFWDTPYT | SEQ ID NO: 119 |

TABLE 9-4
The CDR sequences of the huP2-G9 antibodies
(according to the Chothia numbering scheme)

| Antibody | Name | Sequence | No. |
|---|---|---|---|
| huP2-G9 | HCDR1 | GSSITSGY | SEQ ID NO: 265 |
| | HCDR2 | SYDGS | SEQ ID NO: 266 |
| | HCDR3 | EGAHYSGTLSWFAY | SEQ ID NO: 121 |
| | LCDR1 | RASENIYSNLA | SEQ ID NO: 114 |
| | LCDR2 | AATNLAD | SEQ ID NO: 109 |
| | LCDR3 | QHFWDTPYT | SEQ ID NO: 119 |

### 3. Humanization Engineering of M P2-E8

For humanized antibodies of the M P2-E8 antibody, IGKV1-33*01 was selected as a light chain framework region template, and IGHV3-21*01 was selected as a heavy chain framework region template. The CDRs of the murine antibodies were grafted onto the selected humanization templates, and embedded residues, residues directly interacting with the CDRs, and sites with high-risk hotspots and immunogenicity risks were identified using MOE software and back-mutated and point-mutated to design different light and heavy chain variable regions for humanized antibodies.

TABLE 10
The specific framework replacements and amino acid
substitutions for P2-E8 humanized antibodies

| VL | | VH | |
|---|---|---|---|
| L1 | Graft(IGKV1-33*01) | H1 | Graft(IGHV3-21*01) |
| L2 | Graft(IGKV1-33*01) + | H2 | Graft(IGHV3-21*01) + |
| | A43S, T85V | | S49A |
| L3 | Graft(IGKV1-33*01) + | H3 | Graft(IGHV3-21*01) + |
| | K39R, K42Q, A43S, T85V | | S49A + S55N |
| | | H4 | Graft(IGHV3-21*01) + |
| | | | S49A + I57H |
| FR4 | IGKJ4*01 | FR4 | IGHJ1*01 |

Note:
The amino acids are numbered according to the Kabat numbering scheme.

TABLE 11
The CDRs of P2-E8 humanized antibodies

| Variable | | | SEQ ID |
|---|---|---|---|
| region | CDR | Sequence | NO |
| huP2-E8 VH3 | HCDR2 | YISSGSNIINYADTVKG | 188 |
| huP2-E8 VH4 | HCDR2 | YISSGSSIHNYADTVKG | 189 |

The variable region sequences of P2-E8 humanized antibodies are shown below:

> huP2-E8 VH1
(SEQ ID NO: 190)
EVQLVESGGGLVKPGGSLRLSCAASGFTFSDYGIHWVRQAPGKGLEWVSYISSGS
SIINYADTVKGRFTISRDNAKNSLYLQMNSLRAEDTAVYYCARGPNYYGSSYLLV
YWGQGTLVTVSS
> huP2-E8 VH2
(SEQ ID NO: 191)
EVQLVESGGGLVKPGGSLRLSCAASGFTFSDYGIHWVRQAPGKGLEWVAYISSGS
SIINYADTVKGRFTISRDNAKNSLYLQMNSLRAEDTAVYYCARGPNYYGSSYLLV
YWGQGTLVTVSS
> huP2-E8 VH3
(SEQ ID NO: 192)
EVQLVESGGGLVKPGGSLRLSCAASGFTFSDYGIHWVRQAPGKGLEWVAYISSGS
NIINYADTVKGRFTISRDNAKNSLYLQMNSLRAEDTAVYYCARGPNYYGSSYLLV
YWGQGTLVTVSS
> huP2-E8 VH4
(SEQ ID NO: 193)
EVQLVESGGGLVKPGGSLRLSCAASGFTFSDYGIHWVRQAPGKGLEWVAYISSGS
SIHNYADTVKGRFTISRDNAKNSLYLQMNSLRAEDTAVYYCARGPNYYGSSYLLV
YWGQGTLVTVSS
> huP2-E8 VL1
(SEQ ID NO: 194)
DIQMTQSPSSLSASVGDRVTITCKASQDVNIAVAWYQQKPGKAPKLLIYSASYRY
TGVPSRFSGSGSGTDFTFTISSLQPEDIATYYCQQHYNIPWTFGGGTKVEIK
> huP2-E8 VL2
(SEQ ID NO: 195)
DIQMTQSPSSLSASVGDRVTITCKASQDVNIAVAWYQQKPGKSPKLLIYSASYRYT
GVPSRFSGSGSGTDFTFTISSLQPEDIAVYYCQQHYNIPWTFGGGTKVEIK
> huP2-E8 VL3
(SEQ ID NO: 196)
DIQMTQSPSSLSASVGDRVTITCKASQDVNIAVAWYQQRPGQSPKLLIYSASYRYT
GVPSRFSGSGSGTDFTFTISSLQPEDIAVYYCQQHYNIPWTFGGGTKVEIK
Note: The variable regions are single underlined,
and the CDRs are double underlined; the
CDRs are defined according to the Kabat numbering scheme.

TABLE 12
Humanized antibodies of the P2-E8 series

| | huP2-E8 | huP2-E8 | huP2-E8 | huP2-E8 |
|---|---|---|---|---|
| | VH1 | VH2 | VH3 | VH4 |
| huP2-E8 | P2-E8H1L1 | P2-E8H2L1 | P2-E8H3L1 | P2-E8H4L1 |
| VL1 | | | | (also known |
| | | | | as huP2-E8) |
| huP2-E8 | P2-E8H1L2 | P2-E8H2L2 | P2-E8H3L2 | P2-E8H4L2 |
| VL2 | | | | |
| huP2-E8 | P2-E8H1L3 | P2-E8H2L3 | P2-E8H3L3 | P2-E8H4L3 |
| VL3 | | | | |

### 4. Humanization Engineering of M P1-G7

For humanized antibodies of the M P1-G7 antibody, IGKV1-39*01 was selected as a light chain framework region template, and IGHV4-31*02 was selected as a heavy chain framework region template. The CDRs of the murine antibodies were grafted onto the selected humanization templates, and embedded residues, residues directly interacting with the CDRs, and sites with high-risk hotspots and immunogenicity risks were identified using

MOE software and back-mutated and point-mutated to design different light and heavy chain variable regions for humanized antibodies.

TABLE 13
The specific framework replacements and amino acid
substitutions for P1-G7 humanized antibodies

| VL | | VH | |
|---|---|---|---|
| L1 | Graft(IGKV1-39*01) + | H1 | Graft(IGHV4-31*02) + G27Y, |
| | I48L | | S30T, V71R + Q1E |
| L2 | Graft(IGKV1-39*01) + | H2 | Graft(IGHV4-31*02) + G27Y, |
| | A43S, I48L, F71Y | | S30T, G42E, G44K, V71R + |
| | | | Q1E |
| FR4 | IGKJ4*01 | FR4 | IGHJ6*01 |

Note:
The amino acids are numbered according to the Kabat numbering scheme.

The variable region sequences of P1-G7 humanized antibodies are shown below:

> huP1-G7 VH1
(SEQ ID NO: 197)
EVQLQESGPGLVKPSQTLSLTCTVSGYSITSGYYWNWIRQHPGKGLEWIGYISYD
GGNNYNPFLKNRVTISRDTSKNQFSLKLSSVTAADTAVYYCAREGGSSWEYYGM
DYWGQGTTVTVSS
> huP1-G7 VH2
(SEQ ID NO: 198)
EVQLQESGPGLVKPSQTLSLTCTVSGYSITSGYYWNWIRQHPEKKLEWIGYISYD
GGNNYNPFLKNRVTISRDTSKNQFSLKLSSVTAADTAVYYCAREGGSSWEYYGM
DYWGQGTTVTVSS
> huP1-G7 VL1
(SEQ ID NO: 199)
DIQMTQSPSSLSASVGDRVTITCRASENIYRNLAWYQQKPGKAPKLLLYVAATNLA
DGVPSRFSGSGSGTDFTLTISSLQPEDFATYYCQQFWGSPYTFGGGTKVEIK
> huP1-G7 VL2
(SEQ ID NO: 200)
DIQMTQSPSSLSASVGDRVTITCRASENIYRNLAWYQQKPGKSPKLLLYAATNLA
DGVPSRFSGSGSGTDYTLTISSLQPEDFATYYCQQFWGSPYTFGGGTKVEIK
Note: The variable regions are single underlined,
and the CDRs are double underlined; the
CDRs are defined according to the Kabat numbering scheme.

TABLE 14
Humanized antibodies of the P1-G7 series

| | huP1-G7 VH1 | huP1-G7 VH2 |
|---|---|---|
| huP1-G7 VL1 | P1-G7H1L1 (also known as | P1-G7H2L1 |
| | huP1-G7) | |
| huP1-G7 VL2 | P1-G7H1L2 | P1-G7H2L2 |

### 5. Humanization Engineering of M P2-C6

For humanized antibodies of the P2-C6 antibody, IGKV4-1*01 and IGKV3-11*01 were selected as light chain framework region templates, and IGHV1-18*01 was selected as a heavy chain framework region template. The CDRs of the murine antibodies were grafted onto the selected humanization templates, and embedded residues, residues directly interacting with the CDRs, and sites with high-risk hotspots and immunogenicity risks were identified using MOE software and back-mutated and point-mutated to design different light and heavy chain variable regions for humanized antibodies.

TABLE 15
The specific framework replacements and amino acid
substitutions for P2-C6 humanized antibodies

| VL | | VH | |
|---|---|---|---|
| L1 | Graft(IGKV4-1*01) + | H1 | Graft(IGHV1-18*01) + Q1E, T71V, |
| | M4L, V58I | | T73K, A93T |
| L2 | Graft(IGKV4-1*01) + | H2 | Graft(IGHV1-18*01) + Q1E, Q43E, |
| | M4L, V58I, G68R, V85T | | M69L, T71V, T73K, S76N, A93T |
| L3 | Graft(IGKV3-11*01) + | H3 | Graft(IGHV1-18*01) + Q1E, Q43E, |
| | A43P, R45K, G68R | | M69L, T71V, T73K, S76N, A93T + N57S |
| | | H4 | Graft(IGHV1-18*01) + Q1E, Q43E, |
| | | | M69L, T71V, T73K, S76N, A93T + |
| | | | N57T |
| | | H5 | Graft(IGHV1-18*01) + Q1E, Q43E, |
| | | | M69L, T71V, T73K, S76N, A93T + |
| | | | N54T, N57S |
| | | H6 | Graft(IGHV1-18*01) + Q1E, Q43E, |
| | | | M69L, T71V, T73K, S76N, A93T + |
| | | | N54S |
| | | H7 | Graft(IGHV1-18*01) + Q1E, T71V, |
| | | | T73K, A93T + N54T, N57S |
| FR4 | IGKJ4*01 | FR4 | IGHJ6*01 |

Note:
The amino acids are numbered according to the Kabat numbering scheme.

TABLE 16
The CDRs of P2-C6 humanized antibodies

| Variable | | | SEQ |
|---|---|---|---|
| region | CDR | Sequence | ID NO |
| huP2-C6 VH3 | HCDR2 | YIYPDNGYSGYNQKFKG | 201 |
| huP2-C6 VH4 | HCDR2 | YIYPDNGYTGYNQKFKG | 202 |
| huP2-C6 VH5 | HCDR2 | YIYPDTGYSGYNQKFKG | 203 |
| huP2-C6 VH6 | HCDR2 | YIYPDSGYNGYNQKFKG | 204 |
| huP2-C6 VH7 | HCDR2 | YIYPDTGYSGYNQKFKG | 203 |

The variable region sequences of P2-C6 humanized antibodies are shown below:

> huP2-C6 VH1
(SEQ ID NO: 205)
EVQLVQSGAEVKKPGASVKVSCKASGYTFTDYYIHWVRQAPGQGLEWMG
YIYPDNGYNGYNQKFKGRVTMTVDKSTSTAYMELRSLRSDDTAVYYCTR
RGDYSGVHFDYWGQGTTVTVSS
> huP2-C6 VH2
(SEQ ID NO: 206)
EVQLVQSGAEVKKPGASVKVSCKASGYTFTDYYIHWVRQAPGEGLEWMG
YIYPDNGYNGYNQKFKGRVTLTVDKSTNTAYMELRSLRSDDTAVYYCTR
RGDYSGVHFDYWGQGTTVTVSS
> huP2-C6 VH3
(SEQ ID NO: 207)
EVQLVQSGAEVKKPGASVKVSCKASGYTFTDYYIHWVRQAPGEGLEWMG
YIYPDNGYSGYNQKFKGRVTLTVDKSTNTAYMELRSLRSDDTAVYYCTR
RGDYSGVHFDYWGQGTTVTVSS
> huP2-C6 VH4
(SEQ ID NO: 208)
EVQLVQSGAEVKKPGASVKVSCKASGYTFTDYYIHWVRQAPGEGLEWMG
YIYPDNGYTGYNQKFKGRVTLTVDKSTNTAYMELRSLRSDDTAVYYCTR
RGDYSGVHFDYWGQGTTVTVSS
> huP2-C6 VH5
(SEQ ID NO: 209)
EVQLVQSGAEVKKPGASVKVSCKASGYTFTDYYIHWVRQAPGEGLEWMG
YIYPDTGYSGYNQKFKGRVTLTVDKSTNTAYMELRSLRSDDTAVYYCTR
RGDYSGVHFDYWGQGTTVTVSS
> huP2-C6 VH6
(SEQ ID NO: 210)
EVQLVQSGAEVKKPGASVKVSCKASGYTFTDYYIHWVRQAPGEGLEWMG
YIYPDSGYNGYNQKFKGRVTLTVDKSTNTAYMELRSLRSDDTAVYYCTR
RGDYSGVHFDYWGQGTTVTVSS
> huP2-C6 VH7
(SEQ ID NO: 211)
EVQLVQSGAEVKKPGASVKVSCKASGYTFTDYYIHWVRQAPGQGLEWMG
YIYPDTGYSGYNQKFKGRVTMTVDKSTSTAYMELRSLRSDDTAVYYCTR
RGDYSGVHFDYWGQGTTVTVSS
> huP2-C6 VL1
(SEQ ID NO: 212)
DIVLTQSPDSLAVSLGERATINCRASESVNNYGVGELHWYQQKPGQPPK
LLIYRASTLESGIPDRFSGSGSGTDFTLTISSLQAEDVAVYYCQHSYKD
PPWTFGGGTKVEIK
> huP2-C6 VL2
(SEQ ID NO: 213)
DIVLTQSPDSLAVSLGERATINCRASESVNNYGVGFLHWYQQKPGQPPK
LLIYRASTLESGIPDRFSGSGSRTDFTLTISSLQAEDVATYYCQHSYKD
PPWTFGGGTKVEIK
> huP2-C6 VL3
(SEQ ID NO: 214)
EIVLTQSPATLSLSPGERATLSCRASESVNNYGVGFLHWYQQKPGQPPK
LLIYRASTLESGIPARFSGSGSRTDFTLTISSLEPEDFAVYYCQHSYKD
PPWTFGGGTKVEIK
Note:
The variable regions are single underlined, and
the CDRs are double underlined; the CDRs are
defined according to the Kabat numbering scheme.

TABLE 17-1
Humanized antibodies of the P2-C6 series

| | huP2-C6 | huP2-C6 | huP2-C6 | huP2-C6 | huP2-C6 | huP2-C6 | huP2-C6 |
|---|---|---|---|---|---|---|---|
| | VH1 | VH2 | VH3 | VH4 | VH5 | VH6 | VH7 |
| huP2- | P2- | P2- | P2- | P2- | P2- | P2- | P2- |
| C6 | C6H1L1 | C6H2L1 | C6H3L1 | C6H4L1 | C6H5L1 | C6H6L1 | C6H7L1 |
| VL1 | | | | | | | (also |
| | | | | | | | known as |
| | | | | | | | huP2-C6) |
| huP2- | P2- | P2- | P2- | P2- | P2- | P2- | P2- |
| C6 | C6H1L2 | C6H2L2 | C6H3L2 | C6H4L2 | C6H5L2 | C6H6L2 | C6H7L2 |
| VL2 | | | | | | | |
| huP2- | P2- | P2- | P2- | P2- | P2- | P2- | P2- |
| C6 | C6H1L3 | C6H2L3 | C6H3L3 | C6H4L3 | C6H5L3 | C6H6L3 | C6H7L3 |
| VL3 | | | | | | | |

TABLE 17-2
The CDR sequences of the huP2-C6 antibodies
(according to the Kabat numbering scheme)

| Antibody | Name | Sequence | No. |
|---|---|---|---|
| huP2-C6 | HCDR1 | DYYIH | SEQ ID NO: 135 |
| | HCDR2 | YIYPDTGYSGYNQKFKG | SEQ ID NO: 203 |
| | HCDR3 | RGDYSGVHFDY | SEQ ID NO: 137 |
| | LCDR1 | RASESVNNYGVGFLH | SEQ ID NO: 138 |
| | LCDR2 | RASTLES | SEQ ID NO: 139 |
| | LCDR3 | QHSYKDPPWT | SEQ ID NO: 140 |

TABLE 17-3
The CDR sequences of the huP2-C6 antibodies
(according to the IMGT numbering scheme)

| Antibody | Name | Sequence | No. |
|---|---|---|---|
| huP2-C6 | HCDR1 | GYTFTDYY | SEQ ID NO: 267 |
| | HCDR2 | IYPDTGYS | SEQ ID NO: 268 |
| | HCDR3 | TRRGDYSGVHFDY | SEQ ID NO: 269 |
| | LCDR1 | ESVNNYGVGF | SEQ ID NO: 270 |
| | LCDR2 | RAS | SEQ ID NO: 271 |
| | LCDR3 | QHSYKDPPWT | SEQ ID NO: 140 |

TABLE 17-4
The CDR sequences of the huP2-C6 antibodies
(according to the Chothia numbering scheme)

| Antibody | Name | Sequence | No. |
|---|---|---|---|
| huP2-C6 | HCDR1 | GYTFTDY | SEQ ID NO: 272 |
| | HCDR2 | YPDTGY | SEQ ID NO: 273 |
| | HCDR3 | RGDYSGVHFDY | SEQ ID NO: 137 |
| | LCDR1 | RASESVNNYGVGFLH | SEQ ID NO: 138 |
| | LCDR2 | RASTLES | SEQ ID NO: 139 |
| | LCDR3 | QHSYKDPPWT | SEQ ID NO: 140 |

### 6. Humanization Engineering of M P1-D7

For humanized antibodies of the M P1-D7 antibody, IGKV4-1*01 and IGKV2-28*01 were selected as light chain framework region templates, and IGHV1-18*01 was selected as a heavy chain framework region template. The CDRs of the murine antibodies were grafted onto the selected humanization templates, and embedded residues, residues directly interacting with the CDRs, and sites with high-risk hotspots and immunogenicity risks were identified using MOE software and back-mutated and point-mutated to design different light and heavy chain variable regions for humanized antibodies.

TABLE 18
The specific framework replacements and amino acid
substitutions for P1-D7 humanized antibodies

| VL | | VH | |
|---|---|---|---|
| L1 | Graft(IGKV4-1*01) + | H1 | Graft(IGHV1-18*01) + Q1E, D61E, C102S |
| | P43S, I48L | | |
| L2 | Graft(IGKV2-28*01) + | H2 | Graft(IGHV1-18*01) + V2I, S76T + Q1E, |
| | I48L | | D61E, C102S |
| | | H3 | Graft(IGHV1-18*01) + V2I, G44A, E46K, |
| | | | S76T + Q1E, D61E, C102S |
| | | H4 | Graft(IGHV1-18*01) + V2I, G44A, E46K, |
| | | | S76T + Q1E, C102S |
| | | H5 | Graft(IGHV1-18*01) + V2I, G44A, E46K, |
| | | | S76T + Q1E |
| FR4 | IGKJ4*01 | FR4 | IGHJ6*01 |

Note:
The amino acids are numbered according to the Kabat numbering scheme.

TABLE 19
The CDRs of P1-D7 humanized antibodies

| Variable | | | SEQ |
|---|---|---|---|
| region | CDR | Sequence | ID NO |
| huP1-D7 VH1 | HCDR2 | WINTYSGVPTYAEDFKG | 215 |
| huP1-D7 VH2 | HCDR2 | WINTYSGVPTYAEDFKG | 215 |
| huP1-D7 VH3 | HCDR2 | WINTYSGVPTYAEDFKG | 215 |
| huP1-D7 VH1 | HCDR3 | RTDYDGYVMDS | 216 |
| huP1-D7 VH2 | HCDR3 | RTDYDGYVMDS | 216 |
| huP1-D7 VH3 | HCDR3 | RTDYDGYVMDS | 216 |
| huP1-D7 VH4 | HCDR3 | RTDYDGYVMDS | 216 |

The variable region sequences of P1-D7 humanized antibodies are shown below:

> huP1-D7 VH1
(SEQ ID NO: 217)
EVQLVQSGAEVKKPGASVKVSCKASGYTFTTYGMNWVRQAPGQGLEWMG
WINTYSGVPTYAEDFKGRVTMTTDTSTSTAYMELRSLRSDDTAVYYCAR
RTDYDGYVMDSWGQGTTVTVSS
> huP1-D7 VH2
(SEQ ID NO: 218)
EIQLVQSGAEVKKPGASVKVSCKASGYTFTTYGMNWVRQAPGQGLEWMG
WINTYSGVPTYAEDFKGRVTMTTDTSTTTAYMELRSLRSDDTAVYYCAR
RTDYDGYVMDSWGQGTTVTVSS
> huP1-D7 VH3
(SEQ ID NO: 219)
EIQLVQSGAEVKKPGASVKVSCKASGYTFTTYGMNWVRQAPGQALKWMG
WINTYSGVPTYAEDFKGRVTMTTDTSTTTAYMELRSLRSDDTAVYYCAR
RTDYDGYVMDSWGQGTTVTVSS
> huP1-D7 VH4
(SEQ ID NO: 220)
EIQLVQSGAEVKKPGASVKVSCKASGYTFTTYGMNWVRQAPGQALKWMG
WINTYSGVPTYADDFKGRVTMTTDTSTTTAYMELRSLRSDDTAVYYCAR
RTDYDGYVMDSWGQGTTVTVSS
> huP1-D7 VH5
(SEQ ID NO: 221)
EIQLVQSGAEVKKPGASVKVSCKASGYTFTTYGMNWVRQAPGQALKWMG
WINTYSGVPTYADDFKGRVTMTTDTSTTTAYMELRSLRSDDTAVYYCAR
RTDYDGYVMDCWGQGTTVTVSS
> huP1-D7 VL1
(SEQ ID NO: 222)
DIVMTQSPDSLAVSLGERATINCKSSQSLFYSGNQKNYLAWYQQKPGQS
PKLLLYWASTRESGVPDRFSGSGSGTDFTLTISSLQAEDVAVYYCQQYF
SYPPTFGGGTKVEIK
> huP1-D7 VL2
(SEQ ID NO: 223)
DIVMTQSPLSLPVTPGEPASISCKSSQSLFYSGNQKNYLAWYLQKPGQS
PQLLLYWASTRESGVPDRFSGSGSGTDFTLKISRVEAEDVGVYYCQQYF
SYPPTFGGGTKVEIK
Note:
The variable regions are single underlined, and
the CDRs are double underlined; the CDRs are
defined according to the Kabat numbering scheme.

TABLE 20
Humanized antibodies of the P1-D7 series

| | huP1-D7 | huP1-D7 | huP1-D7 | huP1-D7 | huP1-D7 |
|---|---|---|---|---|---|
| | VH1 | VH2 | VH3 | VH4 | VH5 |
| huP1-D7 | P1-D7H1L1 | P1-D7H2L1 | P1-D7H3L1 | P1-D7H4L1 | P1-D7H5L1 |
| VL1 | (also known as | | | | |
| | huP1-D7) | | | | |
| huP1-D7 | P1-D7H1L2 | P1-D7H2L2 | P1-D7H3L2 | P1-D7H4L2 | P1-D7H5L2 |
| VL2 | | | | | |

### 7. Engineering of P2 Fully Human-Derived Antibody

According to a high-risk hotspot, the point mutation N30Q was added to the VL. According to sites with a risk of immunogenicity, the point mutations T76S and A81E were added to the VL.

TABLE 21
The CDRs of the P2 fully human-derived antibody

| Variable region | CDR | Sequence | SEQ ID NO |
|---|---|---|---|
| VL1 | LCDR1 | QASQDIQNYLN | 224 |

> P2m HCVR
(SEQ ID NO: 163)
QVQLVQSGAEVKKPGSSVKVSCKASGYTFTSYDISWVRQAPGQGLEWMG
GIIPIFGTANYAQKFQGRVTITADESTSTAYMELSSLRSEDTAVYYCTA
GIFLATAYWGQGTLVTVSS
> P2m LCVR
(SEQ ID NO: 225)
DIQMTQSPSSMSASVGDRVTISCQASQDIQNYLNWYQQRPGQAPKLLIY
DASTLETGVPSRFSGSGSGTYFTLTISSLQPEDVATYYCQQYENVPITF
GQGTRLEIK
Note:
The variable regions are single underlined, and
the CDRs are double underlined; the CDRs are
defined according to the Kabat numbering scheme.

### 8. Construction and Expression of Complete Forms of Anti-Human ILT4 Humanized Antibodies

Primers were designed, and VH/VK gene fragments of the humanized antibodies were constructed by PCR. The fragments were then homologously recombined with an expression vector pTT5 (with a signal peptide and a constant region gene (CH1-FC/CL) fragment, constructed in the laboratory) to construct an antibody full-length expression vector VH-CH1-Fc-pTT5/VK-CL-pTT5. Through assays using SPR and FACS methods of binding to ILT4 and the like, the heavy and light chain sequences of the humanized antibodies huP2-E3, huP2-G9, huP2-E8, huP1-G7, huP2-C6, and huP1-D7 and the fully human-derived antibody P2m were finally determined as follows:

The humanized antibody huP2-E3:
The heavy chain (SEQ ID NO: 226):
EVQLVQSGAEVKKPGASVKVSCKASGYTFTDYYMNWVKQAPGQRLEWMGDIN
PNSGGTSYNQKFKGRVTITVDKSGSTAYMELSSLRSEDTAVYYCARGGVTTVVAT
YWYFDVWGQGTTVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEAAGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLGK
The light chain (SEQ ID NO: 227):
DIQMTQSPSSLSASVGDRVTITCRASENIYSNLAWYQQKPGKSPKLLVYAATNVA
DGVPSRFSGSGSGTDYTLTISSLQPEDFATYYCQQFWDTPFTFGQGTKLEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC.
The humanized antibody huP2-G9:
The heavy chain (SEQ ID NO: 228):
EVQLQESGPGLVKPSQTLSLTCTVSGSSITSGYYWNWIRQHPGKGLEWIGYLSYD
GSNNYNPSLKNLVTISRDTSKNQFSLKLSSVTAADTAVYYCAREGAHYSGTLSWF
AYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHN
HYTQKSLSLSPGK
The light chain (SEQ ID NO: 229):
DIQMTQSPSSLSASVGDRVTITCRASENIYSNLAWYQQKPGKSPKLLVYAATNLA
DGVPSRFSGSGSGTDYTLTISSLQPEDFATYYCQHFWDTPYTFGGGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC.
The humanized antibody huP2-E8:
The heavy chain (SEQ ID NO: 230):
EVQLVESGGGLVKPGGSLRLSCAASGFTFSDYGIHWVRQAPGKGLEWVAYISSGS
SIHNYADTVKGRFTISRDNAKNSLYLQMNSLRAEDTAVYYCARGPNYYGSSYLLV
YWGQGTLVTVSSASTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRV
ESKYGPPCPPCPAPEAAGGPSVFLFPPKPKDTLMISRTPEVTCVVVDVSQEDPEVQ
FNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHY
TQKSLSLSLGK
The light chain (SEQ ID NO: 231):
DIQMTQSPSSLSASVGDRVTITCKASQDVNIAVAWYQQKPGKAPKLLIYSASYRY
TGVPSRFSGSGSGTDFTFTISSLQPEDIATYYCQQHYNIPWTFGGGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC.
The humanized antibody huP1-G7:
The heavy chain (SEQ ID NO: 232):
EVQLQESGPGLVKPSQTLSLTCTVSGYSITSGYYWNWIRQHPGKGLEWIGYISYD
GGNNYNPFLKNRVTISRDTSKNQFSLKLSSVTAADTAVYYCAREGGSSWEYYGM
DYWGQGTTVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKR
VESKYGPPCPPCPAPEAAGGPSVFLFPPKPKDTLMISRTPEVTCVVVDVSQEDPEV
QFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNH
YTQKSLSLSLGK
The light chain (SEQ ID NO: 233):
DIQMTQSPSSLSASVGDRVTITCRASENIYRNLAWYQQKPGKAPKLLLYAATNLA
DGVPSRFSGSGSGTDFTLTISSLQPEDFATYYCQQFWGSPYTFGGGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC.
The humanized antibody huP2-C6:
The heavy chain (SEQ ID NO: 234):
EVQLVQSGAEVKKPGASVKVSCKASGYTFTDYYIHWVRQAPGQGLEWMGYIYP
DTGYSGYNQKFKGRVTMTVDKSTSTAYMELRSLRSDDTAVYYCTRRGDYSGVH
FDYWGQGTTVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKK
VEPKSCDKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALH
NHYTQKSLSLSPGK
The light chain (SEQ ID NO: 235):
DIVLTQSPDSLAVSLGERATINCRASESVNNYGVGFLHWYQQKPGQPPKLLIYRA
STLESGIPDRFSGSGSGTDFTLTISSLQAEDVAVYYCQHSYKDPPWTFGGGTKVEI
KRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC.
The humanized antibody huP1-D7:
The heavy chain (SEQ ID NO: 236):
EVQLVQSGAEVKKPGASVKVSCKASGYTFTTYGMNWVRQAPGQGLEWMGWIN
TYSGVPTYAEDFKGRVTMTTDTSTSTAYMELRSLRSDDTAVYYCARRTDYDGYV
MDSWGQGTTVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDK
RVESKYGPPCPPCPAPEAAGGPSVFLFPPKPKDTLMISRTPEVTCVVVDVSQEDPE
VQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHN
HYTQKSLSLSLGK
The light chain (SEQ ID NO: 237):
DIVMTQSPDSLAVSLGERATINCKSSQSLFYSGNQKNYLAWYQQKPGQSPKLLLY
WASTRESGVPDRFSGSGSGTDFTLTISSLQAEDVAVYYCQQYFSYPPTFGGGTKVE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC.
The fully human-derived antibody P2m:
The heavy chain (SEQ ID NO: 238):
QVQLVQSGAEVKKPGSSVKVSCKASGYTFTSYDISWVRQAPGQGLEWMGGIIPIF
GTANYAQKFQGRVTITADESTSTAYMELSSLRSEDTAVYYCTAGIFLATAYWGQGT
LVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKT
HTCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSL
SLSPGK
The light chain (SEQ ID NO: 239):
DIQMTQSPSSMSASVGDRVTISCQASQDIQNYLNWYQQRPGQAPKLLIYDASTLE
TGVPSRFSGSGSGTYFTLTISSLQPEDVATYYCQQYENVPITFGQGTRLEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
Note:
The variable regions are single underlined, and the hIgG1
(LALA), hIgG4 (LALA), and CL constant regions are not
underlined.

The heavy and light chain constant region sequences of the humanized antibodies are shown below:

> hIgG1(LALA)
(SEQ ID NO: 240)
ASTKGPSVFPLAPSSKSTSGGTAALGCLVKDYFPEPVTVSWNSGALTSGVHTFPAV

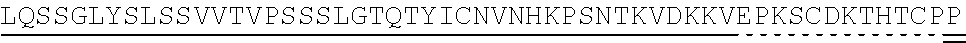

CPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVESCSVMHEALHNHYTQKSLSLSPG
K
Note: The CHI region is single underlined, the hinge region is dotted
underlined, and the Fc region is double underlined.
> hIgG4(LALA)
(SEQ ID NO: 171)
ASTKGPSVFPLAPCSRSTSESTAALGCLVKDYFPEPVTVSWNSGALTSGVHTFPAV

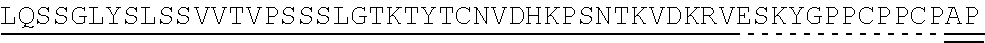

EAAGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSLGK
Note: The CHI region is single underlined, the hinge region is dotted
underlined, and the Fc region is double underlined.
> CL
(SEQ ID NO: 172)
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC

The positive control molecules used in the present disclosure were MK and JTX. The source of the sequences of MK: WO2018187518A1

The amino acid sequences of the theoretical light and heavy chains of MK are shown below (the variable regions are single underlined, the CDRs are double underlined, and the constant regions are dotted underlined):

> The heavy chain
(SEQ ID NO: 241):
EVQLQQWGAGLLKPSETLSLTCAVYGGSFSGYYWSWIRQPPGKGLEWIGEINHA
GSTNYNPSLKSRVTISVDTSKNQFSLKLSSVTAADTAVYYCARLPTRWVTTRYFD

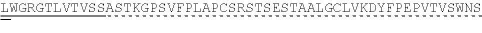

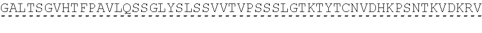

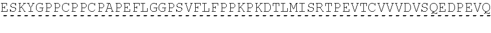

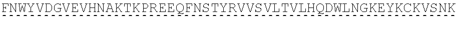

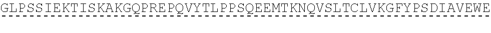

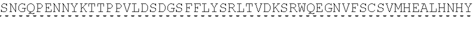

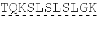

> The light chain
(SEQ ID NO: 242):
ESVLTQPPSVSGAPGQRVTISCTGSSSNIGAGYDVHWYQQLPGTAPKLLIYGDSNR
PSGVPDRFSVSKSGASASLAITGLQAEDEADYYCQSFDNSLSAYVFGGGTQLTVL

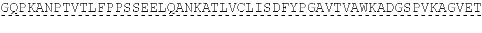

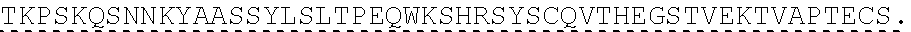

The source of the sequences of JTX: WO2019126514A2

The amino acid sequences of the theoretical light and heavy chains of JTX are shown below (the variable regions are single underlined, the CDRs are double underlined, and the constant regions are dotted underlined):

> The heavy chain
(SEQ ID NO: 243):
QITLKESGPTLVKPTQTLTLTCTFSGFSLNTYAMGVSWIRQPPGKALEWLASIWW
NGNKYNNPSLKSRLTVTKDTSKNQVVLTMTNMDPVDTATYYCAHSRIIRFTDYV

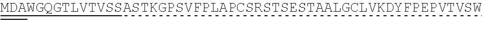

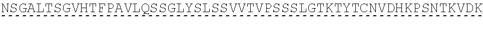

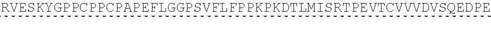

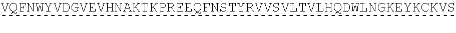

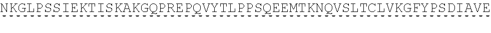

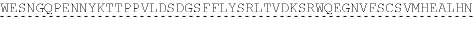

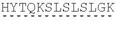

> The light chain
(SEQ ID NO: 244):
DIQMTQSPSSLSTSVGDRVTITCRASEDIYNDLAWYQQKPGKAPKLLIYNANSLH

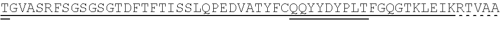

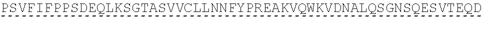

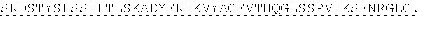

Cells overexpressing HLA-G
(SEQ ID NO: 247)
MVVMAPRTLFLLLSGALTLTETWAGSHSMRYFSAAVSRPGRGEPRFIAMGYVDDTQ
FVRFDSDSACPRMEPRAPWVEQEGPEYWEEETRNTKAHAQTDRMNLQTLRGYYNQS
EASSHTLQWMIGCDLGSDGRLLRGYEQYAYDGKDYLALNEDLRSWTAADTAAQISKRK
CEAANVAEQRRAYLEGTCVEWLHRYLENGKEMLQRADPPKTHVTHHPVFDYEATLRC
WALGFYPAEIILTWQRDGEDQTQDVELVETRPAGDGTFQKWAAVVVPSGEEQRYTCH

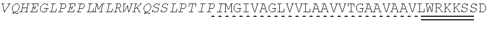

Note: The signal peptide is single underlined,
the extracellular domain is italicized, the
transmembrane domain is dotted underlined, and
the intracellular domain is double underlined.

### Example 5: ELISA Protein Binding Assays of Anti-ILT4 Antibodies

Human ILT4-His protein was diluted to 1 μg/mL with PBS, and a 96-well plate (Corning, CLS3590-100EA) was coated with the diluted protein and incubated overnight at 4° C. The plate was washed and then blocked with 1% BSA at 37° C. for 1 h. The plate was washed, and the anti-ILT4 antibodies, at different concentrations, were then added. The plate was incubated at 37° C. for 1 h. The plate was washed, and an HRP-goat anti-human F(ab′) 2 secondary antibody (Jackson, 109-036-097) was then added. The plate was incubated at 37° C. for 1 h. The plate was washed, and the TMB chromogenic substrate (KPL, 52-00-03) was then added. The plate was incubated at room temperature for 5-10 min, and 1 M H₂SO₄ was added to stop the reaction. The absorbance was measured at a wavelength of 450 nm using a VERSAmax microplate reader (Molecular Devices). The results are shown in Table 22.

TABLE 22
The abilities of the anti-ILT4 antibodies to bind to ILT4 protein

| | | |
|---|---|---|
| Antibody | EC₅₀ (ng/mL) | Top |

Assay 1

| | | |
|---|---|---|
| ChP1-C4 | 64.26 | 1.983 |
| JTX | 82.63 | 1.697 |

Assay 2

| | | |
|---|---|---|
| ChP2-E7 | 72.90 | 2.004 |
| ChP2-G4 | 62.09 | 1.829 |
| ChP2-E11 | 42.80 | 1.984 |
| JTX | 82.63 | 1.697 |

Assay 3

| | | |
|---|---|---|
| ChP1-D7 | 29.23 | 2.071 |
| JTX | 29.34 | 1.426 |

Assay 4

| | | |
|---|---|---|
| ChP1-E11 | 40.96 | 1.722 |
| JTX | 82.97 | 1.225 |

Assay 5

| | | |
|---|---|---|
| ChP2-B9 | 48.66 | 1.530 |
| ChP2-C2 | 49.28 | 1.157 |
| ChP2-E10 | 65.82 | 1.646 |
| JTX | 80.10 | 1.144 |

The experimental results show that all the anti-ILT4 chimeric antibodies exhibited higher abilities to bind to ILT4 protein than the control antibody JTX.

### Example 6: Biacore Protein Binding Assays of Anti-ILT4 Antibodies

The affinities of the humanized antibodies of the present disclosure for ILT4-His protein were determined by Biacore. The antibodies were affinity-captured using a Protein A biosensor chip (Cytiva, 29127556), and ILT4-His was then allowed to flow through the surface of the chip. Reaction signals were detected in real time using an instrument to obtain binding and dissociation curves. After the dissociation in each experimental cycle was complete, the biosensor chip was washed with 10 mM Glycine-HCl pH 1.5 (Cytiva, BR-1003-54) for regeneration. Data fitting was performed using a 1:1 model. The binding and dissociation of each antibody are shown in Table 23.

TABLE 23
The binding affinities of the anti-ILT4 antibodies for ILT4 protein

| Antibody | ka (1/Ms) | kd (1/s) | KD (M) |
|---|---|---|---|
| huP2-E3 | 1.01E+06 | 1.27E−03 | 1.26E−09 |
| huP2-G9 | 9.99E+05 | 1.09E−04 | 1.09E−10 |
| huP2-E8 | 2.22E+05 | 1.86E−04 | 8.39E−10 |
| huP1-G7 | 4.57E+05 | 7.20E−05 | 1.58E−10 |
| huP2-C6 | 1.21E+06 | 6.58E−05 | 5.43E−11 |
| huP1-D7 | 2.59E+06 | 1.03E−02 | 3.98E−09 |
| P2m | 2.03E+06 | 8.78E−05 | 4.33E−11 |
| MK | 4.52E+05 | 8.63E−03 | 1.91E−08 |

The experimental results show that the humanized and fully human-derived antibodies to which the present disclosure relates exhibited higher abilities to bind to ILT4 protein than the control antibody.

### Example 7: FACS Cell Binding Assays of Anti-ILT4 Antibodies

The affinities of the humanized antibodies of the present disclosure for CHO-S/ILT4 cells were determined by FACS. 1×10⁶ cells/mL CHO-S/ILT4 cells were blocked with 1% BSA and then incubated with the anti-ILT4 antibodies, at different concentrations, at 4° C. for 1 h. The cells were washed twice with 1% BSA and then incubated with a F(ab′)2-goat anti-human IgG Fc secondary antibody (PE, Invitrogen, H10104) at 4° C. for 0.5 h. The cells were washed twice with 1% BSA, and fluorescence signal values were then obtained using FACS. The results are shown in Table 24.

TABLE 24
The abilities of the anti-ILT4 antibodies to bind
to the ILT4 stably transfected cell strain

| | | |
|---|---|---|
| Antibody | EC₅₀ (μg/mL) | Top |

Assay 1

| | | |
|---|---|---|
| huP2-E3 | 0.03067 | 25986 |
| MK | 0.03103 | 25142 |

Assay 2

| | | |
|---|---|---|
| huP2-G9 | 0.04500 | 27255 |
| MK | 0.04151 | 22456 |

Assay 3

| | | |
|---|---|---|
| huP2-C6 | 0.03678 | 29886 |
| MK | 0.30103 | 25142 |

Assay 4

| | | |
|---|---|---|
| P2m | 0.01633 | 20764 |
| MK | 0.01388 | 18465 |

The experimental results show that the humanized and fully human-derived antibodies to which the present disclosure relates exhibited comparable or higher abilities to bind to ILT4-positive cells than the control antibody.

### Example 8: Anti-ILT4 Antibodies Do Not Bind to Other LILR Family Members

Human ILT2-His (prepared by the same method as the ILT4-His in Examples 1 and 2; whose amino acid sequence is set forth in SEQ ID NO: 245), ILT3-His (prepared by the same method as the ILT4-His in Examples 1 and 2; whose amino acid sequence is set forth in SEQ ID NO: 246), ILT4-His (prepared by the same method as in Examples 1 and 2), LILRB5-His (Acro, CDC-H5220), LILRA1-His (Acro, LI1-H82E8), LILRA2-His (Acro, LI2-H82E9), LILRA4-His (SinoBiological, 16058-H08H), LILRA5-His (Acro, LI5-H82E1), LILRA6-His (Acro, LI6-H82E2), or LILRA3-His (Acro, LI3-H82E0) was diluted to 1 μg/mL with PBS, and a 96-well plate (Corning, CLS3590-100EA) was coated with the diluted protein and incubated overnight at 4° C. The plate was washed and then blocked with 1% BSA at 37° C. for 1 h. The plate was washed, and the anti-ILT4 antibodies, at 10 μg/mL, were then added. The plate was then incubated at 37° C. for 1 h. The plate was washed, and an HRP-goat anti-human F(ab′) 2 secondary antibody (Jackson, 109-036-097) was then added. The plate was incubated at 37° C. for 1 h. The plate was washed, and the TMB chromogenic substrate (KPL, 52-00-03) was then added. The plate was incubated at room temperature for 5-10 min, and 1 M H₂SO₄ was added to stop the reaction. The absorbance was measured at a wavelength of 450 nm using a VERSAmax microplate reader (Molecular Devices). The results are shown in Table 25.

His-tagged ILT2 protein extracellular domain:
ILT2-His
(SEQ ID NO: 245)
GHLPKPTLWAEPGSVITQGSPVTLRCQGGQETQEYRLYREKKTALW
ITRIPQELVKKGQFPIPSITWEHAGRYRCYYGSDTAGRSESSDPLE
LVVTGAYIKPTLSAQPSPVVNSGGNVILQCDSQVAFDGFSLCKEGE
DEHPQCLNSQPHARGSSRAIFSVGPVSPSRRWWYRCYAYDSNSPYE
WSLPSDLLELLVLGVSKKPSLSVQPGPIVAPEETLTLQCGSDAGYN
RFVLYKDGERDFLQLAGAQPQAGLSQANFTLGPVSRSYGGQYRCYG
AHNLSSEWSAPSDPLDILIAGQFYDRVSLSVQPGPTVASGENVTLL
CQSQGWMQTFLLTKEGAADDPWRLRSTYQSQKYQAEFPMGPVTSAH
AGTYRCYGSQSSKPYLLTHPSDPLELVVSGPSGGPSSPTTGPTSTS
GPEDQPLTPTGSDPQSGLGRHLGVHHHHHH
Note: The extracellular domain is italicized,
and the His tag is single underlined.
His-tagged ILT3 protein extracellular domain:
ILT3-His
(SEQ ID NO: 246)
QAGPLPKPTLWAEPGSVISWGNSVTIWCQGTLEAREYRLDKEESPA
PWDRQNPLEPKNKARFSIPSMTEDYAGRYRCYYRSPVGWSQPSDPL
ELVMTGAYSKPTLSALPSPLVTSGKSVTLLCQSRSPMDTFLLIKER
AAHPLLHLRSEHGAQQHQAEFPMSPVTSVHGGTYRCFSSHGFSHYL
LSHPSDPLELIVSGSLEDPRPSPTRSVSTAAGPEDQPLMPTGSVPH
SGLRRHWEHHHHHH
Note: The extracellular domain is italicized,
and the His tag is single underlined.

TABLE 25
The abilities of the anti-ILT4 antibodies to bind to LILR family member proteins

| Antibody | ILT2 | ILT3 | ILT4 | LILRB5 | LILRA1 | LILRA2 | LILRA3 | LILRA4 | LILRA5 | LILRA6 |
|---|---|---|---|---|---|---|---|---|---|---|
| huP2-E3 | 0.0541 | 0.0699 | 1.0428 | 0.0523 | 0.0532 | 0.0550 | 0.0476 | 0.0605 | 0.0466 | 0.0470 |
| huP2-G9 | 0.0541 | 0.0564 | 1.7672 | 0.0533 | 0.0457 | 0.0759 | 0.0461 | 0.0674 | 0.0441 | 0.0449 |
| huP2-C6 | 0.0621 | 0.1495 | 2.6914 | 0.0563 | 0.0535 | 0.0565 | 0.0613 | 0.0605 | 0.0598 | 0.0606 |
| huP2-E8 | 0.0569 | 0.0630 | 0.9543 | 0.0489 | 0.0483 | 0.0516 | 0.0467 | 0.0504 | 0.0467 | 0.0461 |
| P2m | 0.0517 | 0.0671 | 1.8701 | 0.0663 | 0.0469 | 0.0448 | 0.0468 | 0.0543 | 0.0459 | 0.0453 |

The experimental results show that none of the humanized and fully human-derived antibodies to which the present disclosure relates bound to other LILR family members.

### Example 9: Blocking Effects of Anti-ILT4 Antibodies on Binding of ILT4 to HLA-G

The cell-blocking activity of the anti-ILT4 antibodies was measured by flow cytometry. 1×10⁶ cells/mL CHO-S/ILT4 cells were blocked with 1% BSA and then incubated with the anti-ILT4 antibodies, diluted with 1% BSA to different concentrations, and an HLA-G tetramer (novoprotein) labeled with CF647 (SIGMA, MX647S100-1KT) at 4° C. for 1 h. After 4 min of centrifugation at 500 g, the supernatant was discarded. The cells were washed twice with 1% BSA, and fluorescence signal values were then obtained using FACS.

TABLE 26
The abilities of the anti-ILT4 antibodies
to block the binding of ILT4 to HLA-G

| | |
|---|---|
| Antibody | IC₅₀ (μg/mL) |

Assay 1

| | |
|---|---|
| huP2-E3 | 0.012 |
| JTX | 0.022 |
| MK | 0.031 |

Assay 2

| | |
|---|---|
| huP2-G9 | 0.085 |
| JTX | 0.120 |
| MK | 0.138 |

Assay 3

| | |
|---|---|
| huP2-C6 | 0.072 |
| JTX | 0.120 |
| MK | 0.138 |

Assay 4

| | |
|---|---|
| P2m | 0.033 |
| JTX | 0.061 |
| MK | 0.092 |

The experimental results show that the antibodies huP2-E3, huP2-G9, huP2-C6, and P2m, to which the present disclosure relates, all effectively blocked the binding of ILT4 to HLA-G, and their blocking activity was stronger than that of the control antibodies MK and JTX.

### Example 10: Blocking Effects of Anti-ILT4 Antibodies on ILT4 and Its Downstream Signaling Pathways

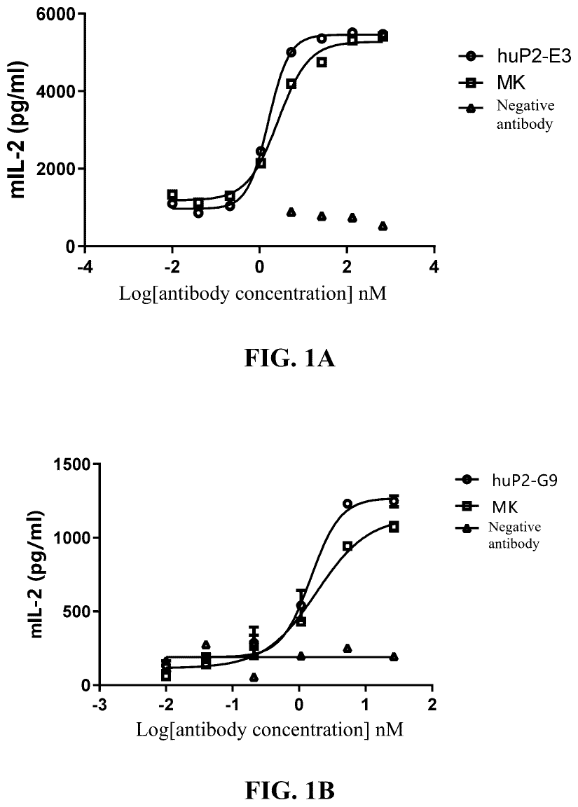

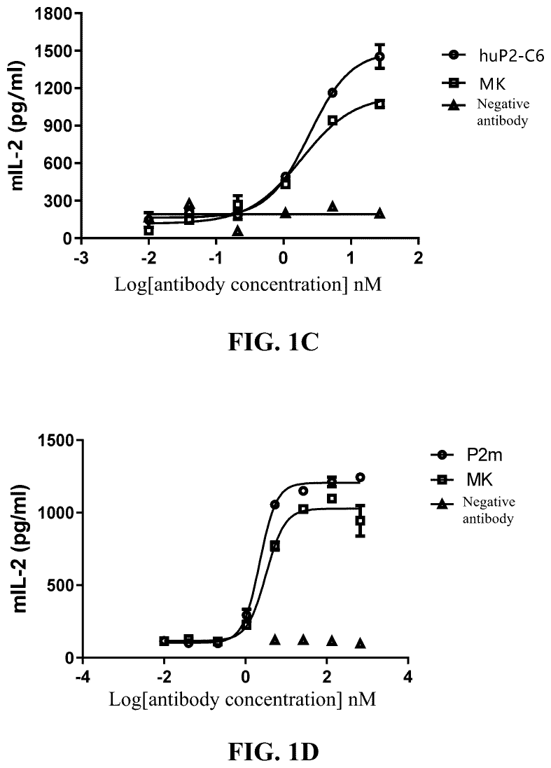

The abilities of the anti-ILT4 antibodies to block ILT4 were determined through a 3A9/ILT4 reporter system. 1 μg/mL mCD3 antibody (BioLegend, 100238) was inoculated into a 96-well plate (Corning, 3599). The plate was incubated at 37° C. for 2 h, and the supernatants were then discarded. 3A9 cells (ATCC, CRL-3293) overexpressing full-length ILT4 protein were collected and resuspended in RPMI 1640 (MeilunBio, PWL004)+5% FBS (Gibco, 10099-141)+0.05 mM 2-B mercaptoethanol (Sigma, M3148-25 mL), and the density was adjusted to 8.3×10⁵ cells/mL. The cell suspension was then added to the above 96-well plate. Meanwhile, the anti-ILT4 antibodies, at different concentrations, were added, and the plate was incubated at 37° C. with 5% CO₂ for 24 h. The cell supernatants were collected after 24 h. mIL-2 secretion was measured using an mIL-2 kit (NeoBioscience, EMC002.96.2). The results are shown in FIGS. 1A-1D.

The experimental results show that all the antibodies to which the present disclosure relates removed the inhibitory effect of ILT4 more effectively and promoted the secretion of higher levels of mIL-2 by 3A9 cells than the control antibody MK.

### Example 11: Effects of Anti-ILT4 Antibodies on Cytokine Secretion by Macrophages

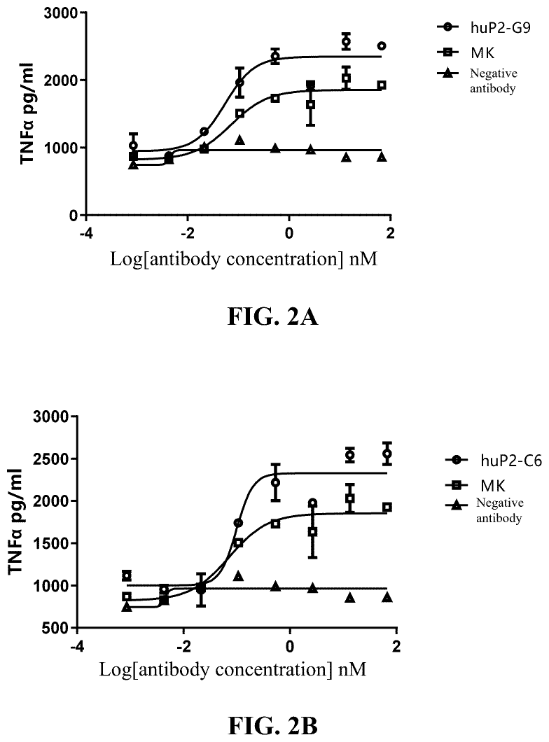

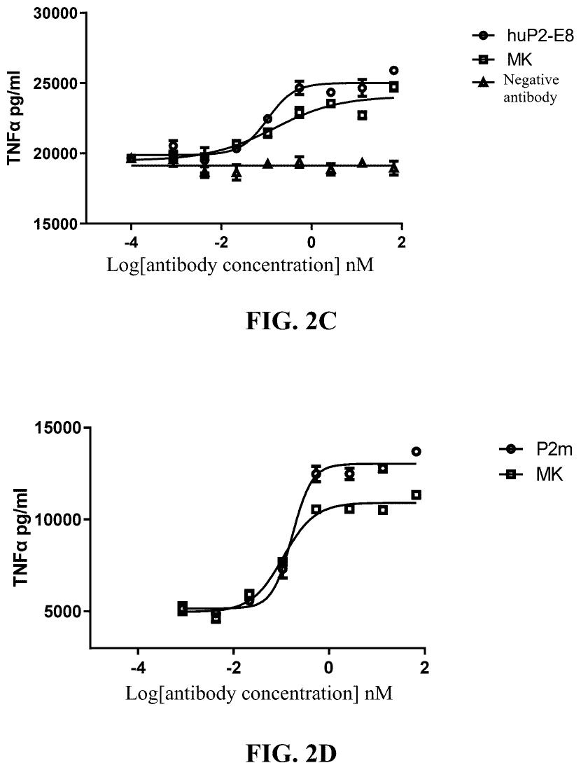

CD14⁺ monocytes were isolated from healthy human PBMCs using CD14 MicroBeads (Miltenyi Biotec, 130-050-201). The CD14⁺ cells were resuspended in RPMI 1640 (MeilunBio, PWL004)+10% FBS (Gibco, 10099-141)+50 ng/ml M-CSF (Peprotech, 300-25), and the cell density was adjusted to 2×10⁶ cells/mL. After 7 days of differentiation, macrophages were obtained. The macrophages were resuspended in RPMI 1640+10% FBS, and the density was adjusted to 1×10⁶ cells/mL. The macrophages were then inoculated into a 96-well plate (Jet, 3788). Meanwhile, LPS (sigma, L3024-5 MG) and the anti-ILT4 antibodies, at different concentrations, were added, and the plate was incubated at 37° C. with 5% CO₂ for 24 h. The cell supernatants were collected and assayed for TNFα using a HUMAN TNFα KIT (Univ, 62HTNFAPEG). The results are shown in FIGS. 2A-2D.

The experimental results show that all the antibodies to which the present disclosure relates more effectively promoted the transformation of macrophages to the M1 phenotype and the secretion of higher levels of the proinflammatory cytokine TNF-α than the control antibody MK.

### Example 12: Combined Effects of Anti-ILT4 Antibodies and Anti-PD-1 Antibody in Allogeneic MLR

CD14⁺ monocytes were isolated from healthy human PBMCs using CD14 MicroBeads (Miltenyi Biotec, 130-050-201). The CD14⁺ cells were resuspended in RPMI 1640 (MeilunBio, PWL004)+10% FBS (Gibco, 10099-141)+50 ng/ml M-CSF (Peprotech, 300-25), and the cell density was adjusted to 2×10⁶ cells/mL. After 7 days of differentiation, macrophages were obtained. The macrophages were resuspended in RPMI 1640+10% FBS, and the density was adjusted to 2×10⁵ cells/mL. The macrophages were then inoculated into a 96-well plate (Jet, 3788).

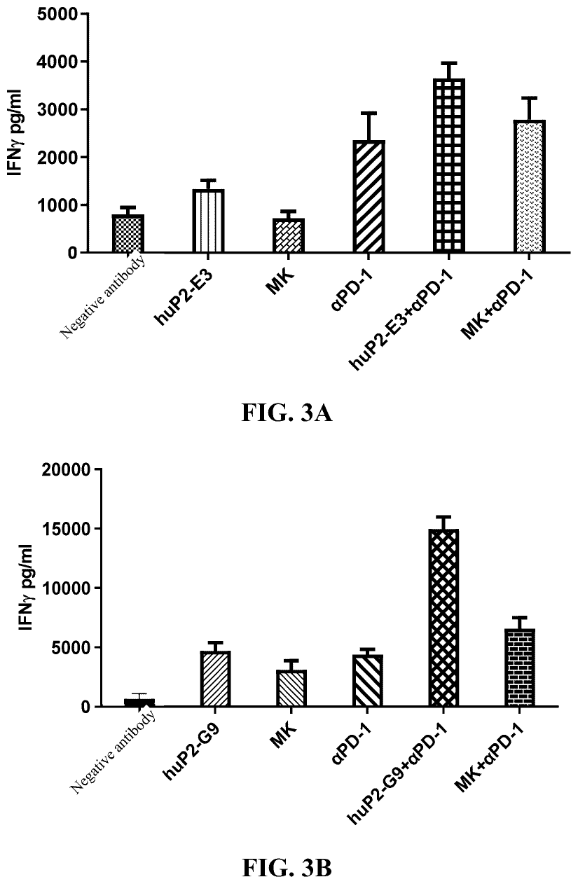

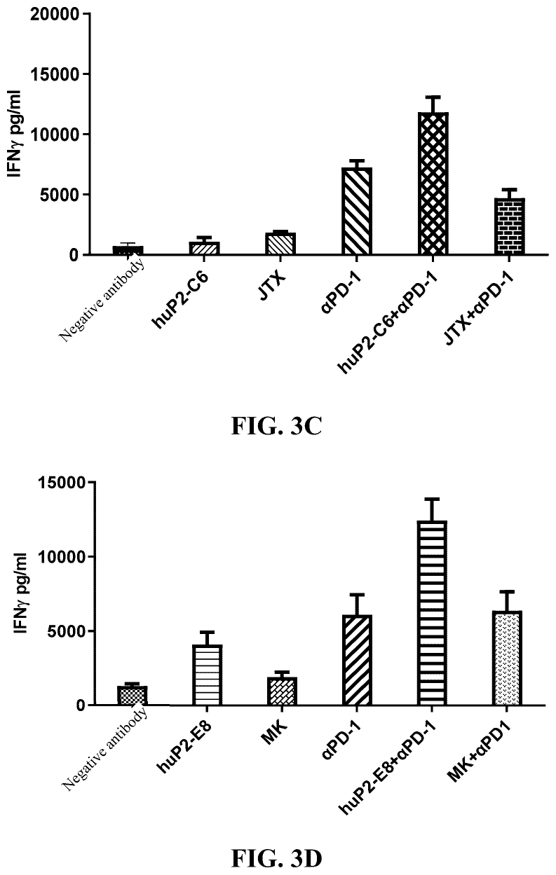

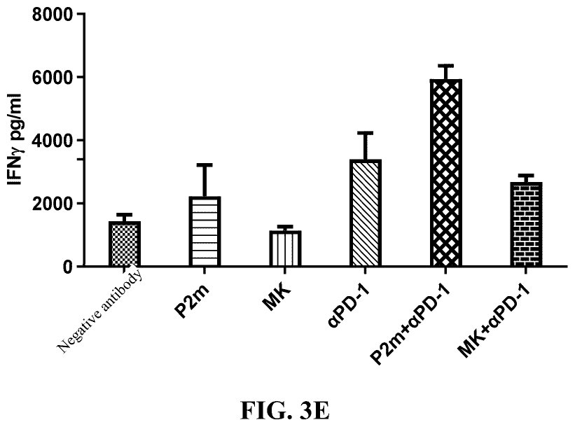

Pan T cells were isolated from PBMCs from another donor using a T cell sorting kit (Miltenyi, 130-096-535) and resuspended in RPMI 1640+10% FBS, and the density was adjusted to 2×10⁶ cells/mL. The cell suspension was then added to the above 96-well plate. Meanwhile, the anti-ILT4 antibodies and/or the anti-PD-1 antibody Hu23-11.IgG4AA (prepared with reference to the patent WO2020156509 A1; its sequences are shown below) were added to a final concentration of 10 μg/mL, and the plate was incubated at 37° C. with 5% CO₂ for 6 d. The cell supernatants were collected and assayed for IFNγ using a HUMAN IFNγ KIT (Univ, 62HIFNGPEG). The results are shown in FIGS. 3A-3E. The full-length sequence of the anti-PD-1 antibody Hu23-11.IgG4AA:

The heavy chain (SEQ ID NO: 248):
EVQLVQSGAEVKKPGSSVKVSCKASGGTFSDYEMHWVRQAPGQGL
EWMGLIDPETGGTVYNQKFKDRVTITADKSTSTAYMELSSLRSED
TAVYYCARERFSYYGSTSDWYFDVWGQGTTVTVSSASTKGPSVFP
LAPCSRSTSESTAALGCLVKDYFPEPVTVSWNSGALTSGVHTFPA
VLQSSGLYSLSSVVTVPSSSLGTKTYTCNVDHKPSNTKVDKRVES
KYGPPCPPCPAPEAAGGPSVFLFPPKPKDTLMISRTPEVTCVVVD
VSQEDPEVQFNWYVDGVEVHNAKTKPREEQFNSTYRVVSVLTVLH
QDWLNGKEYKCKVSNKGLPSSIEKTISKAKGQPREPQVYTLPPSQ
EEMTKNQVSLTCLVKGFYPSDIAVEWESNGQPENNYKTTPPVLDS
DGSFFLYSRLTVDKSRWQEGNVFSCSVMHEALHNHYTQKSLSLSL
GK
The light chain (SEQ ID NO: 249):
DIVMTQTPLSLPVTPGEPASISCRSSQSLVHSTGNTYLEWYLQKP
GQSPQLLIYKVSNRFSGVPDRFSGSGSGTDFTLKISRVEAEDVGV
YYCFQGSHVPYTFGGGTKVEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC

TABLE 27
The CDRs of the
anti-PD-1 antibody Hu23-11.IgG4AA

| | Heavy chain | | Light chain | |
|---|---|---|---|---|
| PD-1 | HCDR1 | DYEMH | LCDR1 | RSSQSLVH |
| | | (SEQ ID | | STGNTYLE |
| | | NO: 250) | | (SEQ ID |
| | | | | NO: 253) |
| | HCDR2 | LIDPETGGT | LCDR2 | KVSNRFS |
| | | VYNQKFKD | | (SEQ ID |
| | | (SEQ ID | | NO: 254) |
| | | NO: 251) | | |
| | HCDR3 | ERFSYYGST | LCDR3 | FQGSHVPY |
| | | SDWYFDV | | T |
| | | (SEQ ID | | (SEQ ID |
| | | NO: 252) | | NO: 255) |

The variable region sequences of the anti-PD-1 antibody Hu23-11.IgG4AA:

PD-1 VH (SEQ ID NO: 256):
EVQLVQSGAEVKKPGSSVKVSCKASGGTFSDYEMHWVRQAPGQGL
EWMGLIDPETGGTVYNQKFKDRVTITADKSTSTAYMELSSLRSED
TAVYYCARERFSYYGSTSDWYFDVWGQGTTVTVSS
PD-1 VL (SEQ ID NO: 257):
DIVMTQTPLSLPVTPGEPASISCRSSQSLVHSTGNTYLEWYLQKP
GQSPQLLIYKVSNRFSGVPDRFSGSGSGTDFTLKISRVEAEDVGV
YYCFQGSHVPYTFGGGTKVEIK.

The experimental results show that in the MLR experiment, the combinations of the antibodies of the present disclosure and the anti-PD-1 antibody were significantly more effective in promoting the release of INFγ than the combination of the positive antibody MK or JTX and the anti-PD-1 antibody.

### Example 13: Roles of Anti-ILT4 Antibodies in Promoting Dendritic Cell Activation

CD14⁺ monocytes were isolated from healthy human PBMCs using CD14 MicroBeads (Miltenyi Biotec, 130-050-201). The CD14⁺ cells were resuspended in RPMI 1640 (MeilunBio, PWL004)+10% FBS (Gibco, 10099-141)+100 ng/mL GM-CSF (Peprotech, 300-03-100 μg)+100 ng/ml IL-4 (R&D, 217-IL-010). After 5-6 days of differentiation, dendritic cells were obtained. The dendritic cell density was adjusted to 1×10⁶ cells/mL, and the dendritic cells were inoculated into a 96-well plate (Jet, 3788). Meanwhile, IL-10 (R&D, 217-IL-010) was added to a final concentration of 10 ng/ml, and the anti-ILT4 antibodies were added to a final concentration of 10 μg/mL. The plate was incubated at 37° C. with 5% CO₂ for 48 h. The cell supernatants were collected and assayed for CCL-2 (NeoBioscience, EHC.113.96.2) and IL-6 (Cisbio, 62HIL06PEG) using an ELISA kit. The upregulation of cytokines or chemokines is shown in Table 28.

TABLE 28
The abilities of the anti-ILT4 antibodies to induce dendritic
cells to secrete proinflammatory cytokines and chemokines

| Antibody | CCL-2 (pg/mL) | IL-6 (pg/mL) |
|---|---|---|
| huP2-G9 | 3446.3863 | 438.9343 |
| MK | 2043.0930 | 322.2405 |
| Negative antibody | 429.1580 | 56.0929 |
| PBS | 593.5481 | 58.7046 |

The experimental results show that in the immunosuppressive environment with IL-10, the humanized antibody huP2-G9, to which the present disclosure relates, still induced dendritic cells to secrete higher levels of proinflammatory cytokines or chemokines.

### Example 14: Inhibitory Effects of Anti-ILT4 Antibodies Against Subcutaneously Grafted Tumors in Mice Bearing Human Breast Cancer MDA-MB-231/HLA-G

HLA-G-overexpressing MDA-MB-231 cells (ATCC, HTB-26) in the logarithmic growth phase (3×10⁶), T cells (5×10⁵), and MØ cells (5×10⁵) were mixed in a ratio of 6:1:1, and 200 μL of the mixture (containing 100 μL of matrigel) was subcutaneously inoculated into the right flank of each mouse (Vital River). Each group contained 6-8 mice. The vehicle control group (the vehicle group) was not inoculated with MØ and T cells. The grouping day was defined as day 0 of the experiment. The stimulation of the remaining PBMCs was stopped, and the culture was continued. After 1 week, the cells were intraperitoneally injected into tumor-bearing mice at 5×10⁶ cells/200 μL/mouse. The anti-ILT4 antibodies were intraperitoneally injected twice a week from day 0 at a dose of 20 mpk. The tumor volume was monitored twice a week, and data were recorded.

The tumor volume of each animal group was expressed as mean±standard error of the mean (Mean±SEM), and plotting was performed using Graphpad Prism 8 software. Statistical analysis was performed using two-way ANOVA.

The calculation formula for tumor volume (V) was: V=½×a×b², where a and b represent length and width, respectively.

Relative tumor proliferation rate T/C (%)=(T−T0)/(C−C0)×100%, where T and C represent the tumor volumes of the treatment group and the control group at the end of the experiment; T0 and C0 represent the tumor volumes at the beginning of the experiment. Tumor growth inhibition rate TGI (%)=1-T/C (%).

The tumor growth inhibition rate of each group is shown in Table 29 below.

TABLE 29
The tumor growth inhibition rate of each group relative to the negative antibody group

| | | | | 7-day | 10-day | 13-day |
|---|---|---|---|---|---|---|
| | | | | tumor | tumor | tumor |
| | Number | | | growth | growth | growth |
| | of | Frequency of | Route of | inhibition | inhibition | inhibition |
| Group | animals | administration | administration | rate % | rate % | rate % |
| PBS vehicle | 8 | biw*3 w | ip | — | — | — |
| control group | | | | | | |
| T + MØ + | 6 | biw*3 w | ip | — | — | — |
| negative | | | | | | |
| antibody-20 | | | | | | |
| mpk | | | | | | |
| T + MØ + MK - | 8 | biw*3 w | ip | 13.3 | 16.0 | 19.6 |
| 20 mpk | | | | | | |
| T + MØ + huP2- | 8 | biw*3 w | ip | 15.3 | 11.8 | 22.2 |
| G9-20 mpk | | | | | | |

Note:
biw stands for “twice a week”, 3 w stands for “three weeks”, and ip stands for “intraperitoneal injection”.

The experimental results show that compared to the PBS vehicle control group (the vehicle group), the tumor growth inhibition rates of the T+MØ+negative antibody-20 mpk group, the MK-20 mpk group, and the huP2-G9-20 mpk group on day 13 were 33.0%, 46.2%, and 47.9%, respectively; compared to the control group, the T+MØ+negative antibody-20 mpk group, the tumor growth inhibition rates of the MK-20 mpk group and the huP2-G9-20 mpk group on day 13 were 19.6% and 22.2%, respectively; the huP2-G9 monotherapy group was comparable in efficacy to the positive MK monotherapy group and even exhibited a higher TGI value.

### Example 15: Inhibitory Effects of Anti-ILT4 Antibodies Against Subcutaneously Grafted Tumors in ILT4-ILT5 Double Transgenic Mice Bearing Mouse Colon Cancer MC38/HLA-G

100 μL of a suspension of MC38 cells (Cobioer, CBP60825) overexpressing HLA-G (the same as in Example 9) (5×10⁵ cells) was subcutaneously inoculated into the right flanks of human ILT4-ILT5 double transgenic mice. Mice with a tumor volume of about 80-100 mm³ were selected and randomly divided into groups of 8. The anti-ILT4 antibodies and the anti-PD-1 antibody (made in-house; the amino acid sequences of the heavy and light chains of the anti-PD-1 antibody (αPD-1) are set forth in SEQ ID NO: 258 and SEQ ID NO: 259, respectively) were intraperitoneally injected twice a week at doses of 30 mpk and 0.3 mpk, respectively. The following day after the grouping, administration was performed, and that day was defined as day 0. The tumor volume was monitored twice a week, and data were recorded.

The tumor volume of each animal group was expressed as mean±standard error of the mean (Mean±SEM), and plotting was performed using Graphpad Prism 8 software. Statistical analysis was performed using two-way ANOVA.

The calculation formula for tumor volume (V) was: V=½×a×b², where a and b represent length and width, respectively.

Relative tumor proliferation rate T/C (%)=(T−T0)/(C−C0)×100%, where T and C represent the tumor volumes of the treatment group and the control group at the end of the experiment; T0 and C0 represent the tumor volumes at the beginning of the experiment. Tumor growth inhibition rate TGI (%)=1-T/C (%).

The tumor growth inhibition rate of each group is shown in Table 30 below.

The heavy chain (SEQ ID NO: 258):
EVRLLESGGGLVKPEGSLKLSCVASGFTFSDYFMSWVRQAPGKGL
EWVAHIYTKSYNYATYYSGSVKGRFTISRDDSRSMVYLQMNNLRT
EDTATYYCTRDGSGYPSLDFWGQGTQVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFLGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLTCLVKGFYPSDIAVEWESNGQPENNYKTTPPVLDSDGSF
FLYSRLTVDKSRWQEGNVFSCSVMHEALHNHYTQKSLSLSLGK
The light chain (SEQ ID NO: 259):
YELTQPPSASVNVGETVKITCSGDQLPKYFADWFHQRSDQTILQV
IYDDNKRPSGIPERISGSSSGTTATLTIRDVRAEDEGDYYCFSGY
VDSDSKLYVFGSGTQLTVLRTVAAPSVFIFPPSDEQLKSGTASVV
CLLNNFYPREAKVQWKVDNALQSGNSQESVTEQDSKDSTYSLSST
LTLSKADYEKHKVYACEVTHQGLSSPVTKSFNRGEC.

TABLE 30
The tumor growth inhibition rate of each group relative to the PBS group

| | | | | 2-day | 4-day | 6-day | 9-day | 11-day | 13-day |
|---|---|---|---|---|---|---|---|---|---|
| | | | | tumor | tumor | tumor | tumor | tumor | tumor |
| | Number | | | growth | growth | growth | growth | growth | growth |
| | of | Frequency of | Route of | inhibition | inhibition | inhibition | inhibition | inhibition | inhibition |
| Group | animals | administration | administration | rate % | rate % | rate % | rate % | rate % | rate % |
| PBS | 8 | biw*3 w | ip | — | — | — | — | — | — |
| MK-30 mpk | 8 | biw*3 w | ip | 23.5 | 27.4 | 12.8 | −6.8 | −0.1 | 9.2 |
| huP2-G9- | 8 | biw*3 w | ip | 50.3 | 38.8 | 27.7 | 14.0 | 30.2 | 31.3 |
| 30 mpk | | | | | | | | | |
| huP2-C6- | 8 | biw*3 w | ip | 58.0 | 43.7 | 35.6 | 17.4 | 31.2 | 29.6 |
| 30 mpk | | | | | | | | | |
| αPD-1- | 8 | biw*3 w | ip | 18.6 | 39.2 | 37.9 | 38.9 | 41.6 | 49.3 |
| 0.3 mpk | | | | | | | | | |
| huP2-G9- | 8 | biw*3 w | ip | 32.6 | 29.9 | 34.9 | 25.3 | 48.1 | 56.3 |
| 30 mpk + αPD- | | | | | | | | | |
| 1-0.3 mpk | | | | | | | | | |
| huP2-C6- | 8 | biw*3 w | ip | 32.4 | 36.2 | 42.7 | 36.7 | 54.1 | 55.1 |
| 30 mpk + αPD- | | | | | | | | | |
| 1-0.3 mpk | | | | | | | | | |

Note:
biw stands for “twice a week”, 3 w stands for “three weeks”, and ip stands for “intraperitoneal injection”.

The experimental results show that compared to the control antibody MK, the huP2-G9 monotherapy and the huP2-C6 monotherapy exhibited relatively good efficacy, and the anti-PD-1 antibody monotherapy exhibited relatively strong efficacy; in addition, huP2-G9 or huP2-C6's respective combination group exhibited slightly stronger efficacy than the corresponding monotherapy group.

Although the foregoing invention has been described in detail using drawings and examples for a clear understanding, the description and examples should not be construed as limiting the scope of the present disclosure. The disclosures of all the patents and scientific literature cited herein are clearly incorporated by reference in their entirety.

1. An anti-ILT4 antibody, comprising a heavy chain variable region and a light chain variable region, wherein the heavy chain variable region comprises a HCDR1, a HCDR2, and a HCDR3, and the light chain variable region comprises a LCDR1, a LCDR2, and a LCDR3, wherein:
a. the HCDR1, the HCDR2, and the HCDR3 of the heavy chain variable region comprise amino acid sequences of a HCDR1, a HCDR2, and a HCDR3, respectively, in SEQ ID NO: 184 or 185, and the LCDR1, the LCDR2, and the LCDR3 of the light chain variable region comprise amino acid sequences of a LCDR1, a LCDR2, and a LCDR3, respectively, in SEQ ID NO: 187 or 186; or
the HCDR1, the HCDR2, and the HCDR3 of the heavy chain variable region comprise amino acid sequences of a HCDR1, a HCDR2, and a HCDR3, respectively, in SEQ ID NO: 32, and the LCDR1, the LCDR2, and the LCDR3 of the light chain variable region comprise amino acid sequences of a LCDR1, a LCDR2, and a LCDR3, respectively, in SEQ ID NO: 33; or
b. the HCDR1, the HCDR2, and the HCDR3 of the heavy chain variable region comprise amino acid sequences of a HCDR1, a HCDR2, and a HCDR3, respectively, in any one of SEQ ID NO: 211, 205, 206, 207, 208, 209, or 210, and the LCDR1, the LCDR2, and the LCDR3 of the light chain variable region comprise amino acid sequences of a LCDR1, a LCDR2, and a LCDR3, respectively, in any one of SEQ ID NO: 212, 213, or 214; or
the HCDR1, the HCDR2, and the HCDR3 of the heavy chain variable region comprise amino acid sequences of a HCDR1, a HCDR2, and a HCDR3, respectively, in SEQ ID NO: 40, and the LCDR1, the LCDR2, and the LCDR3 of the light chain variable region comprise amino acid sequences of a LCDR1, a LCDR2, and a LCDR3, respectively, in SEQ ID NO: 41; or
c. the HCDR1, the HCDR2, and the HCDR3 of the heavy chain variable region comprise amino acid sequences of a HCDR1, a HCDR2, and a HCDR3, respectively, in any one of SEQ ID NO: 181, 176, 177, 178, 179, or 180, and the LCDR1, the LCDR2, and the LCDR3 of the light chain variable region comprise amino acid sequences of a LCDR1, a LCDR2, and a LCDR3, respectively, in SEQ ID NO: 183 or 182; or
the HCDR1, the HCDR2, and the HCDR3 of the heavy chain variable region comprise amino acid sequences of a HCDR1, a HCDR2, and a HCDR3, respectively, in SEQ ID NO: 28, and the LCDR1, the LCDR2, and the LCDR3 of the light chain variable region comprise amino acid sequences of a LCDR1, a LCDR2, and a LCDR3, respectively, in SEQ ID NO: 29; or
d. the HCDR1, the HCDR2, and the HCDR3 of the heavy chain variable region comprise amino acid sequences of a HCDR1, a HCDR2, and a HCDR3, respectively, in any one of SEQ ID NO: 193, 190, 191, or 192, and the LCDR1, the LCDR2, and the LCDR3 of the light chain variable region comprise amino acid sequences of a LCDR1, a LCDR2, and a LCDR3, respectively, in any one of SEQ ID NO: 194, 195, or 196; or
the HCDR1, the HCDR2, and the HCDR3 of the heavy chain variable region comprise amino acid sequences of a HCDR1, a HCDR2, and a HCDR3, respectively, in SEQ ID NO: 10, and the LCDR1, the LCDR2, and the LCDR3 of the light chain variable region comprise amino acid sequences of a LCDR1, a LCDR2, and a LCDR3, respectively, in SEQ ID NO: 11; or
e. the HCDR1, the HCDR2, and the HCDR3 of the heavy chain variable region comprise amino acid sequences of a HCDR1, a HCDR2, and a HCDR3, respectively, in SEQ ID NO: 163, and the LCDR1, the LCDR2, and the LCDR3 of the light chain variable region comprise amino acid sequences of a LCDR1, a LCDR2, and a LCDR3, respectively, in SEQ ID NO: 225 or 164; or
f. the HCDR1, the HCDR2, and the HCDR3 of the heavy chain variable region comprise amino acid sequences of a HCDR1, a HCDR2, and a HCDR3, respectively, in SEQ ID NO: 197 or 198, and the LCDR1, the LCDR2, and the LCDR3 of the light chain variable region comprise amino acid sequences of a LCDR1, a LCDR2, and a LCDR3, respectively, in SEQ ID NO: 199 or 200; or
the HCDR1, the HCDR2, and the HCDR3 of the heavy chain variable region comprise amino acid sequences of a HCDR1, a HCDR2, and a HCDR3, respectively, in SEQ ID NO: 36, and the LCDR1, the LCDR2, and the LCDR3 of the light chain variable region comprise amino acid sequences of a LCDR1, a LCDR2, and a LCDR3, respectively, in SEQ ID NO: 37; or
g. the HCDR1, the HCDR2, and the HCDR3 of the heavy chain variable region comprise amino acid sequences of a HCDR1, a HCDR2, and a HCDR3, respectively, in any one of SEQ ID NO: 217, 218, 219, 220, or 221, and the LCDR1, the LCDR2, and the LCDR3 of the light chain variable region comprise amino acid sequences of a LCDR1, a LCDR2, and a LCDR3, respectively, in SEQ ID NO: 222 or 223; or
the HCDR1, the HCDR2, and the HCDR3 of the heavy chain variable region comprise amino acid sequences of a HCDR1, a HCDR2, and a HCDR3, respectively, in SEQ ID NO: 42, and the LCDR1, the LCDR2, and the LCDR3 of the light chain variable region comprise amino acid sequences of a LCDR1, a LCDR2, and a LCDR3, respectively, in SEQ ID NO: 43; or
h. the HCDR1, the HCDR2, and the HCDR3 of the heavy chain variable region comprise amino acid sequences of a HCDR1, a HCDR2, and a HCDR3, respectively, in SEQ ID NO: 34, and the LCDR1, the LCDR2, and the LCDR3 of the light chain variable region comprise amino acid sequences of a LCDR1, a LCDR2, and a LCDR3, respectively, in SEQ ID NO: 35; or
i. the HCDR1, the HCDR2, and the HCDR3 of the heavy chain variable region comprise amino acid sequences of a HCDR1, a HCDR2, and a HCDR3, respectively, in SEQ ID NO: 18, and the LCDR1, the LCDR2, and the LCDR3 of the light chain variable region comprise amino acid sequences of a LCDR1, a LCDR2, and a LCDR3, respectively, in SEQ ID NO: 19; or
j. the HCDR1, the HCDR2, and the HCDR3 of the heavy chain variable region comprise amino acid sequences of a HCDR1, a HCDR2, and a HCDR3, respectively, in SEQ ID NO: 20, and the LCDR1, the LCDR2, and the LCDR3 of the light chain variable region comprise amino acid sequences of a LCDR1, a LCDR2, and a LCDR3, respectively, in SEQ ID NO: 21; or
k. the HCDR1, the HCDR2, and the HCDR3 of the heavy chain variable region comprise amino acid sequences of a HCDR1, a HCDR2, and a HCDR3, respectively, in SEQ ID NO: 24, and the LCDR1, the LCDR2, and the LCDR3 of the light chain variable region comprise amino acid sequences of a LCDR1, a LCDR2, and a LCDR3, respectively, in SEQ ID NO: 25; or
l. the HCDR1, the HCDR2, and the HCDR3 of the heavy chain variable region comprise amino acid sequences of a HCDR1, a HCDR2, and a HCDR3, respectively, in SEQ ID NO: 44, and the LCDR1, the LCDR2, and the LCDR3 of the light chain variable region comprise amino acid sequences of a LCDR1, a LCDR2, and a LCDR3, respectively, in SEQ ID NO: 45; or
m. the HCDR1, the HCDR2, and the HCDR3 of the heavy chain variable region comprise amino acid sequences of a HCDR1, a HCDR2, and a HCDR3, respectively, in SEQ ID NO: 46, and the LCDR1, the LCDR2, and the LCDR3 of the light chain variable region comprise amino acid sequences of a LCDR1, a LCDR2, and a LCDR3, respectively, in SEQ ID NO: 47; or
n. the HCDR1, the HCDR2, and the HCDR3 of the heavy chain variable region comprise amino acid sequences of a HCDR1, a HCDR2, and a HCDR3, respectively, in SEQ ID NO: 48, and the LCDR1, the LCDR2, and the LCDR3 of the light chain variable region comprise amino acid sequences of a LCDR1, a LCDR2, and a LCDR3, respectively, in SEQ ID NO: 49; or
o. the HCDR1, the HCDR2, and the HCDR3 of the heavy chain variable region comprise amino acid sequences of a HCDR1, a HCDR2, and a HCDR3, respectively, in SEQ ID NO: 50, and the LCDR1, the LCDR2, and the LCDR3 of the light chain variable region comprise amino acid sequences of a LCDR1, a LCDR2, and a LCDR3, respectively, in SEQ ID NO: 51;
preferably,
a. the HCDR1, the HCDR2, and the HCDR3 of the heavy chain variable region comprise amino acid sequences of a HCDR1, a HCDR2, and a HCDR3, respectively, in SEQ ID NO: 184, and the LCDR1, the LCDR2, and the LCDR3 of the light chain variable region comprise amino acid sequences of a LCDR1, a LCDR2, and a LCDR3, respectively, in SEQ ID NO: 187; or
b. the HCDR1, the HCDR2, and the HCDR3 of the heavy chain variable region comprise amino acid sequences of a HCDR1, a HCDR2, and a HCDR3, respectively, in SEQ ID NO: 211, and the LCDR1, the LCDR2, and the LCDR3 of the light chain variable region comprise amino acid sequences of a LCDR1, a LCDR2, and a LCDR3, respectively, in SEQ ID NO: 212; or
c. the HCDR1, the HCDR2, and the HCDR3 of the heavy chain variable region comprise amino acid sequences of a HCDR1, a HCDR2, and a HCDR3, respectively, in SEQ ID NO: 181, and the LCDR1, the LCDR2, and the LCDR3 of the light chain variable region comprise amino acid sequences of a LCDR1, a LCDR2, and a LCDR3, respectively, in SEQ ID NO: 183; or
d. the HCDR1, the HCDR2, and the HCDR3 of the heavy chain variable region comprise amino acid sequences of a HCDR1, a HCDR2, and a HCDR3, respectively, in SEQ ID NO: 193, and the LCDR1, the LCDR2, and the LCDR3 of the light chain variable region comprise amino acid sequences of a LCDR1, a LCDR2, and a LCDR3, respectively, in SEQ ID NO: 194; or
e. the HCDR1, the HCDR2, and the HCDR3 of the heavy chain variable region comprise amino acid sequences of a HCDR1, a HCDR2, and a HCDR3, respectively, in SEQ ID NO: 163, and the LCDR1, the LCDR2, and the LCDR3 of the light chain variable region comprise amino acid sequences of a LCDR1, a LCDR2, and a LCDR3, respectively, in SEQ ID NO: 225; or
f. the HCDR1, the HCDR2, and the HCDR3 of the heavy chain variable region comprise amino acid sequences of a HCDR1, a HCDR2, and a HCDR3, respectively, in SEQ ID NO: 197, and the LCDR1, the LCDR2, and the LCDR3 of the light chain variable region comprise amino acid sequences of a LCDR1, a LCDR2, and a LCDR3, respectively, in SEQ ID NO: 199; or
g. the HCDR1, the HCDR2, and the HCDR3 of the heavy chain variable region comprise amino acid sequences of a HCDR1, a HCDR2, and a HCDR3, respectively, in SEQ ID NO: 217, and the LCDR1, the LCDR2, and the LCDR3 of the light chain variable region comprise amino acid sequences of a LCDR1, a LCDR2, and a LCDR3, respectively, in SEQ ID NO: 222;
more preferably,
a. the HCDR1, the HCDR2, and the HCDR3 of the heavy chain variable region comprise amino acid sequences of a HCDR1, a HCDR2, and a HCDR3, respectively, in SEQ ID NO: 184, and the LCDR1, the LCDR2, and the LCDR3 of the light chain variable region comprise amino acid sequences of a LCDR1, a LCDR2, and a LCDR3, respectively, in SEQ ID NO: 187; or
b. the HCDR1, the HCDR2, and the HCDR3 of the heavy chain variable region comprise amino acid sequences of a HCDR1, a HCDR2, and a HCDR3, respectively, in SEQ ID NO: 211, and the LCDR1, the LCDR2, and the LCDR3 of the light chain variable region comprise amino acid sequences of a LCDR1, a LCDR2, and a LCDR3, respectively, in SEQ ID NO: 212.

2. The anti-ILT4 antibody according to claim 1, wherein:
a. in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119; or
b. in the heavy chain variable region, the HCDR1 comprises an amino acid sequence of SEQ ID NO: 135, the HCDR2 comprises an amino acid sequence of any one of SEQ ID NO: 203, 136, 201, 202, or 204, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140; or
c. in the heavy chain variable region, the HCDR1 comprises an amino acid sequence of SEQ ID NO: 111, the HCDR2 comprises an amino acid sequence of any one of SEQ ID NO: 173, 112, 174, or 175, and the HCDR3 comprises an amino acid sequence of SEQ ID NO: 113; and in the light chain variable region, the LCDR1 comprises an amino acid sequence of SEQ ID NO: 114, the LCDR2 comprises an amino acid sequence of SEQ ID NO: 115, and the LCDR3 comprises an amino acid sequence of SEQ ID NO: 116; or
d. in the heavy chain variable region, the HCDR1 comprises an amino acid sequence of SEQ ID NO: 69, the HCDR2 comprises an amino acid sequence of any one of SEQ ID NO: 189, 70, or 188, and the HCDR3 comprises an amino acid sequence of SEQ ID NO: 71; and in the light chain variable region, the LCDR1 comprises an amino acid sequence of SEQ ID NO: 61, the LCDR2 comprises an amino acid sequence of SEQ ID NO: 62, and the LCDR3 comprises an amino acid sequence of SEQ ID NO: 72; or
e. in the heavy chain variable region, the HCDR1 comprises an amino acid sequence of SEQ ID NO: 165, the HCDR2 comprises an amino acid sequence of SEQ ID NO: 166, and the HCDR3 comprises an amino acid sequence of SEQ ID NO: 167; and in the light chain variable region, the LCDR1 comprises an amino acid sequence of SEQ ID NO: 224 or 168, the LCDR2 comprises an amino acid sequence of SEQ ID NO: 169, and the LCDR3 comprises an amino acid sequence of SEQ ID NO: 170; or
f. in the heavy chain variable region, the HCDR1 comprises an amino acid sequence of SEQ ID NO: 105, the HCDR2 comprises an amino acid sequence of SEQ ID NO: 125, and the HCDR3 comprises an amino acid sequence of SEQ ID NO: 126; and in the light chain variable region, the LCDR1 comprises an amino acid sequence of SEQ ID NO: 127, the LCDR2 comprises an amino acid sequence of SEQ ID NO: 109, and the LCDR3 comprises an amino acid sequence of SEQ ID NO: 128; or
g. in the heavy chain variable region, the HCDR1 comprises an amino acid sequence of SEQ ID NO: 141, the HCDR2 comprises an amino acid sequence of SEQ ID NO: 215 or 142, and the HCDR3 comprises an amino acid sequence of SEQ ID NO: 216 or 143; and in the light chain variable region, the LCDR1 comprises an amino acid sequence of SEQ ID NO: 144, the LCDR2 comprises an amino acid sequence of SEQ ID NO: 145, and the LCDR3 comprises an amino acid sequence of SEQ ID NO: 146; or
h. in the heavy chain variable region, the HCDR1 comprises an amino acid sequence of SEQ ID NO: 99, the HCDR2 comprises an amino acid sequence of SEQ ID NO: 122, and the HCDR3 comprises an amino acid sequence of SEQ ID NO: 123; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 124; or
i. in the heavy chain variable region, the HCDR1 comprises an amino acid sequence of SEQ ID NO: 73, the HCDR2 comprises an amino acid sequence of SEQ ID NO: 87, and the HCDR3 comprises an amino acid sequence of SEQ ID NO: 88; and in the light chain variable region, the LCDR1 comprises an amino acid sequence of SEQ ID NO: 61, the LCDR2 comprises an amino acid sequence of SEQ ID NO: 89, and the LCDR3 comprises an amino acid sequence of SEQ ID NO: 90; or
j. in the heavy chain variable region, the HCDR1 comprises an amino acid sequence of SEQ ID NO: 91, the HCDR2 comprises an amino acid sequence of SEQ ID NO: 92, and the HCDR3 comprises an amino acid sequence of SEQ ID NO: 93; and in the light chain variable region, the LCDR1 comprises an amino acid sequence of SEQ ID NO: 85, the LCDR2 comprises an amino acid sequence of SEQ ID NO: 62, and the LCDR3 comprises an amino acid sequence of SEQ ID NO: 94; or
k. in the heavy chain variable region, the HCDR1 comprises an amino acid sequence of SEQ ID NO: 99, the HCDR2 comprises an amino acid sequence of SEQ ID NO: 100, and the HCDR3 comprises an amino acid sequence of SEQ ID NO: 101; and in the light chain variable region, the LCDR1 comprises an amino acid sequence of SEQ ID NO: 102, the LCDR2 comprises an amino acid sequence of SEQ ID NO: 103, and the LCDR3 comprises an amino acid sequence of SEQ ID NO: 104; or
l. in the heavy chain variable region, the HCDR1 comprises an amino acid sequence of SEQ ID NO: 147, the HCDR2 comprises an amino acid sequence of SEQ ID NO: 148, and the HCDR3 comprises an amino acid sequence of SEQ ID NO: 149; and in the light chain variable region, the LCDR1 comprises an amino acid sequence of SEQ ID NO: 150, the LCDR2 comprises an amino acid sequence of SEQ ID NO: 151, and the LCDR3 comprises an amino acid sequence of SEQ ID NO: 152; or
m. in the heavy chain variable region, the HCDR1 comprises an amino acid sequence of SEQ ID NO: 153, the HCDR2 comprises an amino acid sequence of SEQ ID NO: 154, and the HCDR3 comprises an amino acid sequence of SEQ ID NO: 155; and in the light chain variable region, the LCDR1 comprises an amino acid sequence of SEQ ID NO: 114, the LCDR2 comprises an amino acid sequence of SEQ ID NO: 133, and the LCDR3 comprises an amino acid sequence of SEQ ID NO: 119; or
n. in the heavy chain variable region, the HCDR1 comprises an amino acid sequence of SEQ ID NO: 58, the HCDR2 comprises an amino acid sequence of SEQ ID NO: 156, and the HCDR3 comprises an amino acid sequence of SEQ ID NO: 157; and in the light chain variable region, the LCDR1 comprises an amino acid sequence of SEQ ID NO: 158, the LCDR2 comprises an amino acid sequence of SEQ ID NO: 62, and the LCDR3 comprises an amino acid sequence of SEQ ID NO: 159; or
o. in the heavy chain variable region, the HCDR1 comprises an amino acid sequence of SEQ ID NO: 105, the HCDR2 comprises an amino acid sequence of SEQ ID NO: 160, and the HCDR3 comprises an amino acid sequence of SEQ ID NO: 161; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 162;
preferably,
a. in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119;
b. in the heavy chain variable region, the HCDR1 comprises an amino acid sequence of SEQ ID NO: 135, the HCDR2 comprises an amino acid sequence of SEQ ID NO: 203, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140; or
c. in the heavy chain variable region, the HCDR1 comprises an amino acid sequence of SEQ ID NO: 111, the HCDR2 comprises an amino acid sequence of SEQ ID NO: 173, and the HCDR3 comprises an amino acid sequence of SEQ ID NO: 113; and in the light chain variable region, the LCDR1 comprises an amino acid sequence of SEQ ID NO: 114, the LCDR2 comprises an amino acid sequence of SEQ ID NO: 115, and the LCDR3 comprises an amino acid sequence of SEQ ID NO: 116; or
d. in the heavy chain variable region, the HCDR1 comprises an amino acid sequence of SEQ ID NO: 69, the HCDR2 comprises an amino acid sequence of SEQ ID NO: 189, and the HCDR3 comprises an amino acid sequence of SEQ ID NO: 71; and in the light chain variable region, the LCDR1 comprises an amino acid sequence of SEQ ID NO: 61, the LCDR2 comprises an amino acid sequence of SEQ ID NO: 62, and the LCDR3 comprises an amino acid sequence of SEQ ID NO: 72; or
e. in the heavy chain variable region, the HCDR1 comprises an amino acid sequence of SEQ ID NO: 165, the HCDR2 comprises an amino acid sequence of SEQ ID NO: 166, and the HCDR3 comprises an amino acid sequence of SEQ ID NO: 167; and in the light chain variable region, the LCDR1 comprises an amino acid sequence of SEQ ID NO: 224, the LCDR2 comprises an amino acid sequence of SEQ ID NO: 169, and the LCDR3 comprises an amino acid sequence of SEQ ID NO: 170; or
f. in the heavy chain variable region, the HCDR1 comprises an amino acid sequence of SEQ ID NO: 105, the HCDR2 comprises an amino acid sequence of SEQ ID NO: 125, and the HCDR3 comprises an amino acid sequence of SEQ ID NO: 126; and in the light chain variable region, the LCDR1 comprises an amino acid sequence of SEQ ID NO: 127, the LCDR2 comprises an amino acid sequence of SEQ ID NO: 109, and the LCDR3 comprises an amino acid sequence of SEQ ID NO: 128; or
g. in the heavy chain variable region, the HCDR1 comprises an amino acid sequence of SEQ ID NO: 141, the HCDR2 comprises an amino acid sequence of SEQ ID NO: 215, and the HCDR3 comprises an amino acid sequence of SEQ ID NO: 216; and in the light chain variable region, the LCDR1 comprises an amino acid sequence of SEQ ID NO: 144, the LCDR2 comprises an amino acid sequence of SEQ ID NO: 145, and the LCDR3 comprises an amino acid sequence of SEQ ID NO: 146;
more preferably,
a. in the heavy chain variable region, the HCDR1 comprises an amino acid sequence of SEQ ID NO: 105, the HCDR2 comprises an amino acid sequence of SEQ ID NO: 120, and the HCDR3 comprises an amino acid sequence of SEQ ID NO: 121; and in the light chain variable region, the LCDR1 comprises an amino acid sequence of SEQ ID NO: 114, the LCDR2 comprises an amino acid sequence of SEQ ID NO: 109, and the LCDR3 comprises an amino acid sequence of SEQ ID NO: 119;
b. in the heavy chain variable region, the HCDR1 comprises an amino acid sequence of SEQ ID NO: 135, the HCDR2 comprises an amino acid sequence of SEQ ID NO: 203, and the HCDR3 comprises an amino acid sequence of SEQ ID NO: 137; and in the light chain variable region, the LCDR1 comprises an amino acid sequence of SEQ ID NO: 138, the LCDR2 comprises an amino acid sequence of SEQ ID NO: 139, and the LCDR3 comprises an amino acid sequence of SEQ ID NO: 140.

3. The anti-ILT4 antibody according to claim 1, wherein the anti-ILT4 antibody is a murine antibody, a chimeric antibody, a humanized antibody, or a fully human-derived antibody, preferably a humanized antibody.

4. The anti-ILT4 antibody according to claim 1, wherein:
a. the heavy chain variable region comprises an amino acid sequence having at least 70% sequence identity to SEQ ID NO: 184 or 185, and the light chain variable region comprises an amino acid sequence having at least 70% sequence identity to SEQ ID NO: 187 or 186; or
the heavy chain variable region comprises an amino acid sequence having at least 70% sequence identity to SEQ ID NO: 32, and the light chain variable region comprises an amino acid sequence having at least 70% sequence identity to SEQ ID NO: 33; or
b. the heavy chain variable region comprises an amino acid sequence having at least 70% sequence identity to SEQ ID NO: 211, 205, 206, 207, 208, 209, or 210, and the light chain variable region comprises an amino acid sequence having at least 70% sequence identity to SEQ ID NO: 212, 213, or 214; or
the heavy chain variable region comprises an amino acid sequence having at least 70% sequence identity to SEQ ID NO: 40, and the light chain variable region comprises an amino acid sequence having at least 70% sequence identity to SEQ ID NO: 41; or
c. the heavy chain variable region comprises an amino acid sequence having at least 70% sequence identity to SEQ ID NO: 181, 176, 177, 178, 179, or 180, and the light chain variable region comprises an amino acid sequence having at least 70% sequence identity to SEQ ID NO: 183 or 182; or
the heavy chain variable region comprises an amino acid sequence having at least 70% sequence identity to SEQ ID NO: 28, and the light chain variable region comprises an amino acid sequence having at least 70% sequence identity to SEQ ID NO: 29; or
d. the heavy chain variable region comprises an amino acid sequence having at least 70% sequence identity to SEQ ID NO: 193, 190, 191, or 192, and the light chain variable region comprises an amino acid sequence having at least 70% sequence identity to SEQ ID NO: 194, 195, or 196; or
the heavy chain variable region comprises an amino acid sequence having at least 70% sequence identity to SEQ ID NO: 10, and the light chain variable region comprises an amino acid sequence having at least 70% sequence identity to SEQ ID NO: 11; or
e. the heavy chain variable region comprises an amino acid sequence having at least 70% sequence identity to SEQ ID NO: 163, and the light chain variable region comprises an amino acid sequence having at least 70% sequence identity to SEQ ID NO: 225 or 164;
f. the heavy chain variable region comprises an amino acid sequence having at least 70% sequence identity to SEQ ID NO: 197 or 198, and the light chain variable region comprises an amino acid sequence having at least 70% sequence identity to SEQ ID NO: 199 or 200; or
the heavy chain variable region comprises an amino acid sequence having at least 70% sequence identity to SEQ ID NO: 36, and the light chain variable region comprises an amino acid sequence having at least 70% sequence identity to SEQ ID NO: 37; or
g. the heavy chain variable region comprises an amino acid sequence having at least 70% sequence identity to SEQ ID NO: 217, 218, 219, 220, or 221, and the light chain variable region comprises an amino acid sequence having at least 70% sequence identity to SEQ ID NO: 222 or 223; or
the heavy chain variable region comprises an amino acid sequence having at least 70% sequence identity to SEQ ID NO: 42, and the light chain variable region comprises an amino acid sequence having at least 70% sequence identity to SEQ ID NO: 43;
preferably,
a. the heavy chain variable region comprises an amino acid sequence of SEQ ID NO: 184 or 185, and the light chain variable region comprises an amino acid sequence of SEQ ID NO: 187 or 186; or
the heavy chain variable region comprises an amino acid sequence of SEQ ID NO: 32, and the light chain variable region comprises an amino acid sequence of SEQ ID NO: 33; or
b. the heavy chain variable region comprises an amino acid sequence of SEQ ID NO: 211, 205, 206, 207, 208, 209, or 210, and the light chain variable region comprises an amino acid sequence of SEQ ID NO: 212, 213, or 214; or
the heavy chain variable region comprises an amino acid sequence of SEQ ID NO: 40, and the light chain variable region comprises an amino acid sequence of SEQ ID NO: 41; or
c. the heavy chain variable region comprises an amino acid sequence of SEQ ID NO: 181, 176, 177, 178, 179, or 180, and the light chain variable region comprises an amino acid sequence of SEQ ID NO: 183 or 182; or
the heavy chain variable region comprises an amino acid sequence of SEQ ID NO: 28, and the light chain variable region comprises an amino acid sequence of SEQ ID NO: 29; or
d. the heavy chain variable region comprises an amino acid sequence of SEQ ID NO: 193, 190, 191, or 192, and the light chain variable region comprises an amino acid sequence of SEQ ID NO:
194, 195, or 196; or
the heavy chain variable region comprises an amino acid sequence of SEQ ID NO: 10, and the light chain variable region comprises an amino acid sequence of SEQ ID NO: 11; or
e. the heavy chain variable region comprises an amino acid sequence of SEQ ID NO: 163, and the light chain variable region comprises an amino acid sequence of SEQ ID NO: 225 or 164;
f. the heavy chain variable region comprises an amino acid sequence of SEQ ID NO: 197 or 198, and the light chain variable region comprises an amino acid sequence of SEQ ID NO: 199 or 200;
or
the heavy chain variable region comprises an amino acid sequence of SEQ ID NO: 36, and the light chain variable region comprises an amino acid sequence of SEQ ID NO: 37; or
g. the heavy chain variable region comprises an amino acid sequence of SEQ ID NO: 217, 218, 219, 220, or 221, and the light chain variable region comprises an amino acid sequence of SEQ ID NO: 222 or 223; or
the heavy chain variable region comprises an amino acid sequence of SEQ ID NO: 42, and the light chain variable region comprises an amino acid sequence of SEQ ID NO: 43.

5. The anti-ILT4 antibody according to claim 1, wherein
the anti-ILT4 antibody is an antibody fragment; preferably, the antibody fragment is a Fab, Fab′, F(ab′)2, Fd, Fv, scFv, dsFv, or dAb.

6. The anti-ILT4 antibody according to claim 1, wherein
the anti-ILT4 antibody comprises a heavy chain constant region and a light chain constant region;
preferably, the heavy chain constant region is a human IgG1 or IgG4 heavy chain constant region, and the light chain constant region is a human k light chain constant region;
more preferably, the heavy chain constant region comprises an amino acid sequence of SEQ ID NO: 240 or 171, and the light chain constant region comprises an amino acid sequence of SEQ ID NO: 172.

7. The anti-ILT4 antibody according to claim 6, wherein the anti-ILT4 antibody comprises a heavy chain and a light chain, wherein:
a. the heavy chain comprises an amino acid sequence having at least 70% sequence identity to SEQ ID NO: 228, and the light chain comprises an amino acid sequence having at least 70% sequence identity to SEQ ID NO: 229; or
b. the heavy chain comprises an amino acid sequence having at least 70% sequence identity to SEQ ID NO: 234, and the light chain comprises an amino acid sequence having at least 70% sequence identity to SEQ ID NO: 235; or
c. the heavy chain comprises an amino acid sequence having at least 70% sequence identity to SEQ ID NO: 226, and the light chain comprises an amino acid sequence having at least 70% sequence identity to SEQ ID NO: 227; or
d. the heavy chain comprises an amino acid sequence having at least 70% sequence identity to SEQ ID NO: 230, and the light chain comprises an amino acid sequence having at least 70% sequence identity to SEQ ID NO: 231; or
e. the heavy chain comprises an amino acid sequence having at least 70% sequence identity to SEQ ID NO: 238, and the light chain comprises an amino acid sequence having at least 70% sequence identity to SEQ ID NO: 239; or
f. the heavy chain comprises an amino acid sequence having at least 70% sequence identity to SEQ ID NO: 232, and the light chain comprises an amino acid sequence having at least 70% sequence identity to SEQ ID NO: 233; or
g. the heavy chain comprises an amino acid sequence having at least 70% sequence identity to SEQ ID NO: 236, and the light chain comprises an amino acid sequence having at least 70% sequence identity to SEQ ID NO: 237;
preferably,
a. an amino acid sequence of the heavy chain is set forth in SEQ ID NO: 228, and an amino acid sequence of the light chain is set forth in SEQ ID NO: 229; or
b. an amino acid sequence of the heavy chain is set forth in SEQ ID NO: 234, and an amino acid sequence of the light chain is set forth in SEQ ID NO: 235; or
c. an amino acid sequence of the heavy chain is set forth in SEQ ID NO: 226, and an amino acid sequence of the light chain is set forth in SEQ ID NO: 227; or
d. an amino acid sequence of the heavy chain is set forth in SEQ ID NO: 230, and an amino acid sequence of the light chain is set forth in SEQ ID NO: 231; or
e. an amino acid sequence of the heavy chain is set forth in SEQ ID NO: 238, and an amino acid sequence of the light chain is set forth in SEQ ID NO: 239; or
f. an amino acid sequence of the heavy chain is set forth in SEQ ID NO: 232, and an amino acid sequence of the light chain is set forth in SEQ ID NO: 233; or
g. an amino acid sequence of the heavy chain is set forth in SEQ ID NO: 236, and an amino acid sequence of the light chain is set forth in SEQ ID NO: 237.

8. A pharmaceutical composition, comprising the anti-ILT4 antibody according to claim 1 and one or more pharmaceutically acceptable carriers, diluents, or excipients, wherein preferably, the pharmaceutical composition further comprises at least one second therapeutic agent; more preferably, the second therapeutic agent is an anti-PD-1 antibody.

9. An isolated nucleic acid, wherein the isolated nucleic acid encodes the anti-ILT4 antibody according to claim 1.

10. (canceled)

11. (canceled)

12. (canceled)

13. A method for treating a disease, comprising administering to a subject a therapeutically effective amount of the anti-ILT4 antibody according to claim 1, wherein:
preferably, the disease is a tumor; more preferably, the disease is selected from the group consisting of astrocytoma, glioblastoma, bladder cancer, bone cancer, brain cancer, breast cancer, carcinoid, cervical cancer, choroid plexus papilloma, colorectal cancer, fallopian tube cancer, ependymoma, gallbladder cancer, gastric cancer, head and neck cancer, idiopathic myelofibrosis, renal cancer, renal pelvis tumor, leukemia, liver cancer, esophageal cancer, lung cancer, lymphoma, medulloblastoma, melanoma, meningioma, Merkel cell carcinoma, mesothelioma, multiple myeloma, neuroblastoma, oligodendroglioma, ovarian cancer, peritoneal tumor, pancreatic cancer, polycythemia vera, primary neuroectodermal tumor, prostate cancer, retinoblastoma, salivary gland cancer, sarcoma, small intestine cancer, colorectal cancer, squamous cell carcinoma, thyroid cancer, endometrial cancer, vestibular schwannoma, blastoma, teratoma, pituitary cancer, vulva cancer, thymoma, testicular cancer, bile duct cancer, pheochromocytoma, paraganglioma, and adenoid cystic carcinoma;
most preferably, the disease is selected from the group consisting of ovarian cancer, lung cancer, esophageal cancer, colorectal cancer, renal cell carcinoma, and melanoma.

14. The method for treating a disease according to claim 13, further comprising using a second therapeutic agent, wherein preferably, the second therapeutic agent comprises an immune checkpoint inhibitor; more preferably, the second therapeutic agent is an anti-PD-1 antibody.

15. The method for treating a disease according to claim 14, wherein the anti-PD-1 antibody comprises a heavy chain variable region PD-1-VH and a light chain variable region PD-1-VL, wherein:
the PD-1-VH comprises: a PD-1-HCDR1 comprising an amino acid sequence of SEQ ID NO: 250, a PD-1-HCDR2 comprising an amino acid sequence of SEQ ID NO: 251, and a PD-1-HCDR3 comprising an amino acid sequence of SEQ ID NO: 252, and the PD-1-VL comprises: a PD-1-LCDR1 comprising an amino acid sequence of SEQ ID NO: 253, a PD-1-LCDR2 comprising an amino acid sequence of SEQ ID NO: 254, and a PD-1-LCDR3 comprising an amino acid sequence of SEQ ID NO: 255;
preferably,
the PD-1-VH comprises an amino acid sequence of SEQ ID NO: 256, and the PD-1-VL comprises an amino acid sequence of SEQ ID NO: 257.


SEQUENCE LISTING (273 sequences)

SEQ ID NO: 1 - protein, 672 aa, synthetic construct
  CHAIN 1..672: note = ILT4-Fc
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 446 aa, synthetic construct
  CHAIN 1..446: note = ILT4-His
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVHHHHHH

SEQ ID NO: 3 - protein, 598 aa, synthetic construct
  CHAIN 1..598: note = CHO-S/ILT4
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 4 - protein, 117 aa, Mus musculus
  source 1..117: note = HCVR
  REGION 1..117: note = M P1-B4 HCVR
QVQLKQSGPGLVQPSQSLSITCTVSSFSLTNYGVHWVRQSPGKGLEWLGVIWTGGSTDYNAAFISRLSIIRDNSKSQVFFKMNSLQADDTAMYYCAITTVVTAMDYWGQGTSVTVSS

SEQ ID NO: 5 - protein, 107 aa, Mus musculus
  source 1..107: note = LCVR
  REGION 1..107: note = M P1-B4 LCVR
DIVMTQSHKFMSTSVGDRVSITCKASQDVNIAVVWYQQKPGQSPKLLIYAASYRYTGVPDRFTGSGSGTDFTFTISSVQAEDLAVYYCQQHYSSPYTFGGGTKLEIK

SEQ ID NO: 6 - protein, 117 aa, Mus musculus
  source 1..117: note = HCVR
  REGION 1..117: note = M P1-B9 HCVR
QVQLKQSGPGLVQPSQSLSITCTVSGFSLTSYGVHWVRQSPGKGLEWLGVIWSGGNTDYNAAFMSRLRINKDNSKSQVFFKMNSLQGSDTAIYYCATPYDVVPFAYWGQGTLVTVSS

SEQ ID NO: 7 - protein, 107 aa, Mus musculus
  source 1..107: note = LCVR
  REGION 1..107: note = M P1-B9 LCVR
DIVMTQSHKFMSTSVGDRVSITCKASQDVNIAVAWYQQKPGLSPKLLIYSASYRYTGVPGRFTGSGSGTDFTFTISSVKAEDLAVYYCQQHYSIPFTFGAGTKLEIK

SEQ ID NO: 8 - protein, 115 aa, Mus musculus
  source 1..115: note = HCVR
  REGION 1..115: note = M P1-D10 HCVR
QVQLKQSGPGLVQPSQSLSITCTVSGFSLTTYGVHWVRQSPGKGLEWLGVIWSGGNTDSNAAFISRLSISKDNSKSQVFFKMNNLQADDTAIYYCARYSYAMDYWGQGTSVTVSS

SEQ ID NO: 9 - protein, 107 aa, Mus musculus
  source 1..107: note = LCVR
  REGION 1..107: note = M P1-D10 LCVR
DIVMTQSHKFMSTSVGDRVSITCKASQDVSNGVAWYQQKPGQSPKVLIYAASYRYTGVPDRFTGSGSGTDFTFTIGSVQAEDLALYYCQQHYSTPFTFGSGTMLEIK

SEQ ID NO: 10 - protein, 122 aa, Mus musculus
  source 1..122: note = HCVR
  REGION 1..122: note = M P2-E8 HCVR
EVQLLESGGGLVKPGGSLKLSCAASGFTFSDYGIHWVRQAPEKGLEWVAYISSGSSIINYADTVKGRFTISRDNAKNTLLLQMTSLRSEDTAMYYCARGPNYYGSSYLLVYWGQGTLVTVSS

SEQ ID NO: 11 - protein, 107 aa, Mus musculus
  source 1..107: note = LCVR
  REGION 1..107: note = M P2-E8 LCVR
DIVMTQSHKFMSTSVGDRVSITCKASQDVNIAVAWYQQRPGQSPKLLIYSASYRYTGVPDRFTGSGSGTDFTFTISSVQAEDLAVYYCQQHYNIPWTFGGGTKLEIK

SEQ ID NO: 12 - protein, 120 aa, Mus musculus
  REGION 1..120: note = M P2-C10 HCVR
QVQLQQPGAELVKPGASVKLSCKASGYTFTSYWMHWVKQRPGQGLEWIGVIHPNSDTTNYNEKFKNKATLTVDKSSSTAYMQLSSLTSEDSAVYYCATRYDYAYYAMDYWGQGTSVTVSS

SEQ ID NO: 13 - protein, 107 aa, Mus musculus
  REGION 1..107: note = M P2-C10 LCVR
DIVMTQSHKFMSTSVGDRVSITCKASQDVTTAVAWYQQKPGQSPKLLIYSASYRYTGVPDRFTGSGSGTDFTFTISSVQAEDLAVYHCQQHYSTSPTFGGGTMLEIK

SEQ ID NO: 14 - protein, 120 aa, Mus musculus
  REGION 1..120: note = M P2-F4 HCVR
EVQLVESGGGLVKPGGSLKLSCAASGFTFSDYGMHWVRQAPEKGLEWVAYISSGSRIIYYADTVKGRFTISRDNAKNTLFLQMTSLRSEDTAMYSCARFYNYAHYTMDYWGQGTSVTVSS

SEQ ID NO: 15 - protein, 107 aa, Mus musculus
  REGION 1..107: note = M P2-F4 LCVR
DIVMTQSHKFMSTSVGDRVSITCKASQDVSTAVAWYQQKPGQSPKLLIYSASYRYTGVPDRFTGSGSGTDFTFTISSVQAEDLTLYYCQHHYSTPPTFGGGTKLEIK

SEQ ID NO: 16 - protein, 117 aa, Mus musculus
  REGION 1..117: note = M P2-B3 HCVR
QVQLKQSGPGLVQPSQSLSITCTVSGFSLTNYGVHWFRQSPGKGLEWLGMIWSGGNTDYNAAFTSRLIITKDNSKSQVFFKMNSMQADDTAIYYCARGDYGSSSAYWGQGTLVTVSS

SEQ ID NO: 17 - protein, 107 aa, Mus musculus
  REGION 1..107: note = M P2-B3 LCVR
DIVMTQSHKFMSTSVGDRVSITCKASQDVSIAVAWYQQKPGQSPKLLIYSASYRYTGVPDRFTGSGSGTDFTFTISSVQAEDLAVYYCQQHYSNTLTFGAGTKLEIK

SEQ ID NO: 18 - protein, 121 aa, Mus musculus
  REGION 1..121: note = M P2-E7 HCVR
QVQLQQPGTELVKPGASVKLSCKASGYTFTSYWMHWVKQSPGQGLEWIGNINPSNGGPNYNENFKTKATLTVDKSSSTAYMQLNSLTSEDSAVYYCAGGDYYGGSYLFAYWGQGTLVTVSS

SEQ ID NO: 19 - protein, 107 aa, Mus musculus
  REGION 1..107: note = M P2-E7 LCVR
DTVMTQSHKFMSTSVGDRVSVTCKASQDVNIAVAWYQQKPGQSPKLLIYSSSYRYTGVPDRFTGSGSETDFTFTISSVQAEDLAVYYCQQHYTFPYTFGGGTKLEIK

SEQ ID NO: 20 - protein, 121 aa, Mus musculus
  REGION 1..121: note = M P2-G4 HCVR
EVQLQQSGPELVKPGASVKMSCKASGYTFTDYNMHWVKQSHGKSLEWIGYINPNNGGTTYNQKFKGKATLTVNKSSSTAYMELRSLTSEDSAVYYCASSEYYGSTFLFAYWGQGTLVTVSS

SEQ ID NO: 21 - protein, 107 aa, Mus musculus
  REGION 1..107: note = M P2-G4 LCVR
DIVMTQSHKFMSTSVGDRVGITCKASQDVSIAVAWYQQKPGQSPKLLIYSASYRYTGVPDRFTGSGSGTDFTFTISSVQAEDLAAYYCQQHYSTPWTFGGGTKLEIK

SEQ ID NO: 22 - protein, 116 aa, Mus musculus
  REGION 1..116: note = M P2-C4 HCVR
QVQLKESGPGLVAPSQSLSITCTVSGFSLSTYAIGWVRQPPGKGLEWLGVIWTGGGTSYNSALKSRLSISKDNSKSQVFLKMNSLQTDDTARYYCARGDYGPFAYWGQGTLVTVSS

SEQ ID NO: 23 - protein, 107 aa, Mus musculus
  REGION 1..107: note = M P2-C4 LCVR
DIVMTQSHKFMSTSLGDRVSISCKASQDVSIAVAWYQQKAGQSPKLLIYSASYRHTGVPDRFTGSGSGTDFTFTISSVQAEDLAVYYCQQHYSTPFTFGSGTKLEIK

SEQ ID NO: 24 - protein, 121 aa, Mus musculus
  REGION 1..121: note = M P2-E11 HCVR
QVQLQQSGPELVKPGASVKISCKASAYTFTDYYINWVKQRPGQGLEWIGWIFPGSGSTYYNEKFKGKATLTVDKSSSTAYMLVSSLTSEDSAVYFCARSGTLVAPYAMDYWGQGTSVTVSS

SEQ ID NO: 25 - protein, 107 aa, Mus musculus
  REGION 1..107: note = M P2-E11 LCVR
SIVMTQTPKFLLVSAGDRVTITCKASQSVSNDVAWYQQKPGQSPKLLIYYTSNRYTGVPDRFTGSGYGTDFTFTISTVQAEDLAVYFCQQDYSFPLTFGAGTKLEIK

SEQ ID NO: 26 - protein, 122 aa, Mus musculus
  REGION 1..122: note = M P1-F7 HCVR
DVQLQESGPGLVKPSQSLSLTCSVTGYSITSGYYWNWIRQFPGNKLEWMGYISYDGNNYYNPSLKNRISVTRDTSKNQFFLKLNSVTSEDTATYYCAREDGSYSDYDGFAYWGQGTLVTVSS

SEQ ID NO: 27 - protein, 107 aa, Mus musculus
  REGION 1..107: note = M P1-F7 LCVR
DIQMTQSPASLSVSVGETVTITCRANENIYSNLAWYQQKQGKSPQLLVYAATNLADGVSSRFSGSGSGTQYSLRINSLQSEDFGSYYCQQFWGTPYTFGGGTKLEIK

SEQ ID NO: 28 - protein, 124 aa, Mus musculus
  REGION 1..124: note = M P2-E3 HCVR
EVQLQQSGPELVKPGASVKISCKASGYTFTDYYMNWVKQSHGKSLEWIGDINPNNGGTSYNQKFKGKATLTVDKSSSTAYMELRSLTSEDSTVYYCARGGVTTVVATYWYFDVWGTGTTVTVSS

SEQ ID NO: 29 - protein, 107 aa, Mus musculus
  REGION 1..107: note = M P2-E3 LCVR
DIQMTQSPASLSVSVGETVTITCRASENIYSNLAWYQQKQGKSPQLLVYAATNVADGVPSRFSGGGSGTQYSLKINSLQSEDFGSYYCQQFWDTPFTFGSGTKLEIK

SEQ ID NO: 30 - protein, 122 aa, Mus musculus
  REGION 1..122: note = M P2-F10 HCVR
DVQLQESGPGLVKPSQSLSLTCSVTGYSIASGYYWNWIRQFPGNKLEWMGYITYDGSNNYNPSLKNRISITRDTSKNQFFLKLNSVTTEDTATYYCAREYSYGSTLSWFAYWGQGTLVTVSS

SEQ ID NO: 31 - protein, 107 aa, Mus musculus
  REGION 1..107: note = M P2-F10 LCVR
DIQMTQSPASLSISVGETVTITCRASENIYSNLAWYQQKQGKSPQLLVYAATNLADGVPSRFSGSGSGTQYSLKINSLQSEDFGSYYCQHFWDTPYTFGGGTKLEIK

SEQ ID NO: 32 - protein, 123 aa, Mus musculus
  REGION 1..123: note = M P2-G9 HCVR
DVQLQESGPGLVKPSQSLSLACSVTGSSITSGYYWNWIRQFPGNKLEWMGYLSYDGSNNYNPSLKNRISITRDTSKSQFFLKLNSVTPEDTATYYCAREGAHYSGTLSWFAYWGQGTLVTVSS

SEQ ID NO: 33 - protein, 107 aa, Mus musculus
  REGION 1..107: note = M P2-G9 LCVR
DIQMTQSPASLSVSVGETVTITCRASENIYSNLAWYQQKQGKSPQLLVYAATNLADGVPSRFSGSGSGTQYSLKINSLQSEDFGSYYCQHFWDTPYTFGGGTKLEIK

SEQ ID NO: 34 - protein, 122 aa, Mus musculus
  REGION 1..122: note = M P1-C4 HCVR
QVQLKQSGAELVRPGASVKLSCKASGYSFTDYYINWVKQRPGQGLEWIARIYPGNDNTYYNEKFKDKATLTADSSSSTAYMQLSSLTSEDSAVYFCTRSQDGYYEENAMDYWGQGTSVTVSS

SEQ ID NO: 35 - protein, 107 aa, Mus musculus
  REGION 1..107: note = M P1-C4 LCVR
DIQMTQSPASLSVSVGETVTITCRASENIYSNLAWYQQRQGKSPQLLVYAATNLADGVPFRFSGSGSGTQYSLKINSLQSEDFGSYYCQQFWGSPHTFGGGTKLEIK

SEQ ID NO: 36 - protein, 122 aa, Mus musculus
  REGION 1..122: note = M P1-G7 HCVR
DVQLQESGPGLVKPSQSLSLTCSVTGYSITSGYYWNWIRQFPENKLEWMGYISYDGGNNYNPFLKNRISITRDTSKNQFFLNLNSVTIEDTATYYCAREGGSSWEYYGMDYWGQGTSVTVSS

SEQ ID NO: 37 - protein, 107 aa, Mus musculus
  REGION 1..107: note = M P1-G7 LCVR
DIQMTQSPASLSVSVGETVTITCRASENIYRNLAWYQQKQGHSPQLLLYAATNLADGVPSRFSGSGSGTQYSLKINSLQSEDFGSYYCQQFWGSPYTFGGGTKLEIK

SEQ ID NO: 38 - protein, 124 aa, Mus musculus
  REGION 1..124: note = M P2-C7 HCVR
EVQLQQSGPELVKPGTSVKMSCKASGYTFTDYSMHWVRQSHGKSLEWIGYFNPNNGDTIYNQKFKGKATLTVNKSSSTAYMELRSLTSDDSAVYYCAREDSVSTNAWDGALDYWGQGTSVTVSS

SEQ ID NO: 39 - protein, 107 aa, Mus musculus
  REGION 1..107: note = M P2-C7 LCVR
DIQMTQSPASLSVSVGETVTITCRTSENIYSNLAWYQQKQGKSPQLLVYGATNLADGVPSRFSGSGSGTQYSLKINSLQSEDFGSYYCQHFWDIPWTFGGGTTLEIK

SEQ ID NO: 40 - protein, 120 aa, Mus musculus
  REGION 1..120: note = M P2-C6 HCVR
EVQLQQSGPELVKPGASVKMSCEASGYTFTDYYIHWVKQSHGESLEWIGYIYPDNGYNGYNQKFKGKATLTVDKSSNTAYMELRSLTSEDSAVYFCTRRGDYSGVHFDYWGQGTALTVSS

SEQ ID NO: 41 - protein, 112 aa, Mus musculus
  REGION 1..112: note = M P2-C6 LCVR
DIVLTQSPASLAVSLGQRATISCRASESVNNYGVGFLHWYQQKPGQPPKLLIYRASTLESGIPARFSGSGSRTDFTLTINPVETDDVATYYCQHSYKDPPWTFGGGTKLEIK

SEQ ID NO: 42 - protein, 120 aa, Mus musculus
  REGION 1..120: note = M P1-D7 HCVR
QIQLVQSGPDLKKPGETVKISCKASGYTFTTYGMNWVKQAPGKALKWMGWINTYSGVPTYADDFKGRFAFSLETSATTAYLQINNLKNEDTATYFCARRTDYDGYVMDCWGQGTSVTVSS

SEQ ID NO: 43 - protein, 113 aa, Mus musculus
  REGION 1..113: note = M P1-D7 LCVR
DIVMSQSPSSLAVSIGEKVTMSCKSSQSLFYSGNQKNYLAWYLQKPGQSPKLLLYWASTRESGVPDRFTGSGSGTDFTLTISSVMAEDLAVYYCQQYFSYPPTFGGGTKLEIK

SEQ ID NO: 44 - protein, 125 aa, Mus musculus
  REGION 1..125: note = M P1-E11 HCVR
EVQLQQSGPELVKPGASVKMSCQASGYTFTDYSIHWVKQSRGASLEWIGYINPNNGVTNYNQKFKGKATLTVNKSSSTAYMELRSLTSEDSAVYYCAREGDSVTTIVVHWYFDVGGTGTTVTVSS

SEQ ID NO: 45 - protein, 107 aa, Mus musculus
  REGION 1..107: note = M P1-E11 LCVR
DIQMTQSPASLSVSLGETVTITCRASENIYSNLVWYQQKQGKFPQLLVYAAINLVDGVPSRFSGSGSGTQYSLKINSLQSEDFGSYYCQQFWDTPLTFGAGTKLEIK

SEQ ID NO: 46 - protein, 123 aa, Mus musculus
  REGION 1..123: note = M P2-B9 HCVR
EVQLQQSGPELVKPGASVKISCKASGYTFTDYFMNWVKLSHGKSLEWIGDINPNNGGTSYNQRFKDKVTLTVDKSSSTAYMELRSLTSDSSAVYFCARNGYYDSNYLSYFDVWGTGTTVTVSS

SEQ ID NO: 47 - protein, 107 aa, Mus musculus
  REGION 1..107: note = M P2-B9 LCVR
DIQMTQSPASLSVSVGETVTITCRASENIYSNLAWYQQKQGKSPQLLVYGATNLADGVPSRFSGSGSGTQYSLKINSLQSEDFGSYYCQHFWDTPYTFGGGTKLEIK

SEQ ID NO: 48 - protein, 117 aa, Mus musculus
  REGION 1..117: note = M P2-C2 HCVR
QVQLKQSGPGLVQPSQSLSITCTVSGFSLTSYGVHWVRQSPGKGLEWLGVIWSGGHTDLNAAFISRLSINKDNSKSQVFFKMNSLQGDDTAIYYCARNGYGGCLDYWGQGTTLTVSS

SEQ ID NO: 49 - protein, 107 aa, Mus musculus
  REGION 1..107: note = M P2-C2 LCVR
DIVMTQSHKFMSTSVGDRVSITCKAGQDVSIAVAWYQQKPGQSPQLLIYSASYRYTGVPDRFTGSGSGTDFTFAISSVQAEDLAVYYCQQYYSIPPSFGAGTKLEIK

SEQ ID NO: 50 - protein, 123 aa, Mus musculus
  REGION 1..123: note = M P2-E10 HCVR
DVQLQESGPGLVKPSQSLSLTCSVTGYSITSGYYWNWIRQFPGNKLEWMGYMSYEGTNNSNPSLKNRISITRDTSKNQFFLKLNSVTTEDTARYYCARERAYYYGTLGAMDYWGQGTSVTVSS

SEQ ID NO: 51 - protein, 107 aa, Mus musculus
  REGION 1..107: note = M P2-E10 LCVR
DIQMTQSPASLSVSVGETVTITCRASENIYSNLAWYQQKQGKSPQLLVYAATNLADGVPSRFSGSGSGTQYSLKINSMQSEDFGSYYCQHFWDTPFTFGSGTKLEIK

SEQ ID NO: 52 - protein, 5 aa, Mus musculus
  REGION 1..5: note = M P1-B4-HCDR1
NYGVH

SEQ ID NO: 53 - protein, 16 aa, Mus musculus
  REGION 1..16: note = M P1-B4-HCDR2
VIWTGGSTDYNAAFIS

SEQ ID NO: 54 - protein, 9 aa, Mus musculus
  REGION 1..9: note = M P1-B4-HCDR3
TTVVTAMDY

SEQ ID NO: 55 - protein, 11 aa, Mus musculus
  REGION 1..11: note = M P1-B4-LCDR1
KASQDVNIAVV

SEQ ID NO: 56 - protein, 7 aa, Mus musculus
  REGION 1..7: note = M P1-B4-LCDR2
AASYRYT

SEQ ID NO: 57 - protein, 9 aa, Mus musculus
  REGION 1..9: note = M P1-B4-LCDR3
QQHYSSPYT

SEQ ID NO: 58 - protein, 5 aa, Mus musculus
  REGION 1..5: note = M P1-B9-HCDR1
SYGVH

SEQ ID NO: 59 - protein, 16 aa, Mus musculus
  REGION 1..16: note = M P1-B9-HCDR2
VIWSGGNTDYNAAFMS

SEQ ID NO: 60 - protein, 9 aa, Mus musculus
  REGION 1..9: note = M P1-B9-HCDR3
PYDVVPFAY

SEQ ID NO: 61 - protein, 11 aa, Mus musculus
  REGION 1..11: note = M P1-B9-LCDR1
KASQDVNIAVA

SEQ ID NO: 62 - protein, 7 aa, Mus musculus
  REGION 1..7: note = M P1-B9-LCDR2
SASYRYT

SEQ ID NO: 63 - protein, 9 aa, Mus musculus
  REGION 1..9: note = M P1-B9-LCDR3
QQHYSIPFT

SEQ ID NO: 64 - protein, 5 aa, Mus musculus
  REGION 1..5: note = M P1-D10-HCDR1
TYGVH

SEQ ID NO: 65 - protein, 16 aa, Mus musculus
  REGION 1..16: note = M P1-D10-HCDR2
VIWSGGNTDSNAAFIS

SEQ ID NO: 66 - protein, 7 aa, Mus musculus
  REGION 1..7: note = M P1-D10-HCDR3
YSYAMDY

SEQ ID NO: 67 - protein, 11 aa, Mus musculus
  REGION 1..11: note = M P1-D10-LCDR1
KASQDVSNGVA

SEQ ID NO: 68 - protein, 9 aa, Mus musculus
  REGION 1..9: note = M P1-D10-LCDR3
QQHYSTPFT

SEQ ID NO: 69 - protein, 5 aa, Mus musculus
  REGION 1..5: note = M P2-E8-HCDR1
DYGIH

SEQ ID NO: 70 - protein, 17 aa, Mus musculus
  REGION 1..17: note = M P2-E8-HCDR2
YISSGSSIINYADTVKG

SEQ ID NO: 71 - protein, 13 aa, Mus musculus
  REGION 1..13: note = M P2-E8-HCDR3
GPNYYGSSYLLVY

SEQ ID NO: 72 - protein, 9 aa, Mus musculus
  REGION 1..9: note = M P2-E8-LCDR3
QQHYNIPWT

SEQ ID NO: 73 - protein, 5 aa, Mus musculus
  REGION 1..5: note = M P2-C10-HCDR1
SYWMH

SEQ ID NO: 74 - protein, 17 aa, Mus musculus
  REGION 1..17: note = M P2-C10-HCDR2
VIHPNSDTTNYNEKFKN

SEQ ID NO: 75 - protein, 11 aa, Mus musculus
  REGION 1..11: note = M P2-C10-HCDR3
RYDYAYYAMDY

SEQ ID NO: 76 - protein, 11 aa, Mus musculus
  REGION 1..11: note = M P2-C10-LCDR1
KASQDVTTAVA

SEQ ID NO: 77 - protein, 9 aa, Mus musculus
  REGION 1..9: note = M P2-C10-LCDR3
QQHYSTSPT

SEQ ID NO: 78 - protein, 5 aa, Mus musculus
  REGION 1..5: note = M P2-F4-HCDR1
DYGMH

SEQ ID NO: 79 - protein, 17 aa, Mus musculus
  REGION 1..17: note = M P2-F4-HCDR2
YISSGSRIIYYADTVKG

SEQ ID NO: 80 - protein, 11 aa, Mus musculus
  REGION 1..11: note = M P2-F4-HCDR3
FYNYAHYTMDY

SEQ ID NO: 81 - protein, 11 aa, Mus musculus
  REGION 1..11: note = M P2-F4-LCDR1
KASQDVSTAVA

SEQ ID NO: 82 - protein, 9 aa, Mus musculus
  REGION 1..9: note = M P2-F4-LCDR3
QHHYSTPPT

SEQ ID NO: 83 - protein, 16 aa, Mus musculus
  REGION 1..16: note = M P2-B3-HCDR2
MIWSGGNTDYNAAFTS

SEQ ID NO: 84 - protein, 9 aa, Mus musculus
  REGION 1..9: note = M P2-B3-HCDR3
GDYGSSSAY

SEQ ID NO: 85 - protein, 11 aa, Mus musculus
  REGION 1..11: note = M P2-B3-LCDR1
KASQDVSIAVA

SEQ ID NO: 86 - protein, 9 aa, Mus musculus
  REGION 1..9: note = M P2-B3-LCDR3
QQHYSNTLT

SEQ ID NO: 87 - protein, 17 aa, Mus musculus
  REGION 1..17: note = M P2-E7-HCDR2
NINPSNGGPNYNENFKT

SEQ ID NO: 88 - protein, 12 aa, Mus musculus
  REGION 1..12: note = M P2-E7-HCDR3
GDYYGGSYLFAY

SEQ ID NO: 89 - protein, 7 aa, Mus musculus
  REGION 1..7: note = M P2-E7-LCDR2
SSSYRYT

SEQ ID NO: 90 - protein, 9 aa, Mus musculus
  REGION 1..9: note = M P2-E7-LCDR3
QQHYTFPYT

SEQ ID NO: 91 - protein, 5 aa, Mus musculus
  REGION 1..5: note = M P2-G4-HCDR1
DYNMH

SEQ ID NO: 92 - protein, 17 aa, Mus musculus
  REGION 1..17: note = M P2-G4-HCDR2
YINPNNGGTTYNQKFKG

SEQ ID NO: 93 - protein, 12 aa, Mus musculus
  REGION 1..12: note = M P2-G4-HCDR3
SEYYGSTFLFAY

SEQ ID NO: 94 - protein, 9 aa, Mus musculus
  REGION 1..9: note = M P2-G4-LCDR3
QQHYSTPWT

SEQ ID NO: 95 - protein, 5 aa, Mus musculus
  REGION 1..5: note = M P2-C4-HCDR1
TYAIG

SEQ ID NO: 96 - protein, 16 aa, Mus musculus
  REGION 1..16: note = M P2-C4-HCDR2
VIWTGGGTSYNSALKS

SEQ ID NO: 97 - protein, 8 aa, Mus musculus
  REGION 1..8: note = M P2-C4-HCDR3
GDYGPFAY

SEQ ID NO: 98 - protein, 7 aa, Mus musculus
  REGION 1..7: note = M P2-C4-LCDR2
SASYRHT

SEQ ID NO: 99 - protein, 5 aa, Mus musculus
  REGION 1..5: note = M P2-E11-HCDR1
DYYIN

SEQ ID NO: 100 - protein, 17 aa, Mus musculus
  REGION 1..17: note = M P2-E11-HCDR2
WIFPGSGSTYYNEKFKG

SEQ ID NO: 101 - protein, 12 aa, Mus musculus
  REGION 1..12: note = M P2-E11-HCDR3
SGTLVAPYAMDY

SEQ ID NO: 102 - protein, 11 aa, Mus musculus
  REGION 1..11: note = M P2-E11-LCDR1
KASQSVSNDVA

SEQ ID NO: 103 - protein, 7 aa, Mus musculus
  REGION 1..7: note = M P2-E11-LCDR2
YTSNRYT

SEQ ID NO: 104 - protein, 9 aa, Mus musculus
  REGION 1..9: note = M P2-E11-LCDR3
QQDYSFPLT

SEQ ID NO: 105 - protein, 6 aa, Mus musculus
  REGION 1..5: note = M P1-F7-LCDR1
SGYYWN

SEQ ID NO: 106 - protein, 16 aa, Mus musculus
  REGION 1..16: note = M P1-F7-HCDR2
YISYDGNNYYNPSLKN

SEQ ID NO: 107 - protein, 13 aa, Mus musculus
  REGION 1..13: note = M P1-F7-HCDR3
EDGSYSDYDGFAY

SEQ ID NO: 108 - protein, 11 aa, Mus musculus
  REGION 1..11: note = M P1-F7-LCDR1
RANENIYSNLA

SEQ ID NO: 109 - protein, 7 aa, Mus musculus
  REGION 1..7: note = M P1-F7-LCDR2
AATNLAD

SEQ ID NO: 110 - protein, 9 aa, Mus musculus
  REGION 1..9: note = M P1-F7-LCDR3
QQFWGTPYT

SEQ ID NO: 111 - protein, 5 aa, Mus musculus
  REGION 1..5: note = M P2-E3-HCDR1
DYYMN

SEQ ID NO: 112 - protein, 17 aa, Mus musculus
  REGION 1..17: note = M P2-E3-HCDR2
DINPNNGGTSYNQKFKG

SEQ ID NO: 113 - protein, 15 aa, Mus musculus
  REGION 1..15: note = M P2-E3-HCDR3
GGVTTVVATYWYFDV

SEQ ID NO: 114 - protein, 11 aa, Mus musculus
  REGION 1..11: note = M P2-E3-LCDR1
RASENIYSNLA

SEQ ID NO: 115 - protein, 7 aa, Mus musculus
  REGION 1..7: note = M P2-E3-LCDR2
AATNVAD

SEQ ID NO: 116 - protein, 9 aa, Mus musculus
  REGION 1..9: note = M P2-E3-LCDR3
QQFWDTPFT

SEQ ID NO: 117 - protein, 16 aa, Mus musculus
  REGION 1..16: note = M P2-F10-HCDR2
YITYDGSNNYNPSLKN

SEQ ID NO: 118 - protein, 13 aa, Mus musculus
  REGION 1..13: note = M P2-F10-HCDR3
EYSYGSTLSWFAY

SEQ ID NO: 119 - protein, 9 aa, Mus musculus
  REGION 1..9: note = M P2-F10-LCDR3
QHFWDTPYT

SEQ ID NO: 120 - protein, 16 aa, Mus musculus
  REGION 1..16: note = M P2-G9-HCDR2
YLSYDGSNNYNPSLKN

SEQ ID NO: 121 - protein, 14 aa, Mus musculus
  REGION 1..14: note = M P2-G9-HCDR3
EGAHYSGTLSWFAY

SEQ ID NO: 122 - protein, 17 aa, Mus musculus
  REGION 1..17: note = M P1-C4-HCDR2
RIYPGNDNTYYNEKFKD

SEQ ID NO: 123 - protein, 13 aa, Mus musculus
  REGION 1..13: note = M P1-C4-HCDR3
SQDGYYEENAMDY

SEQ ID NO: 124 - protein, 9 aa, Mus musculus
  REGION 1..9: note = M P1-C4-LCDR3
QQFWGSPHT

SEQ ID NO: 125 - protein, 16 aa, Mus musculus
  REGION 1..16: note = M P1-G7-HCDR2
YISYDGGNNYNPFLKN

SEQ ID NO: 126 - protein, 13 aa, Mus musculus
  REGION 1..13: note = M P1-G7-HCDR3
EGGSSWEYYGMDY

SEQ ID NO: 127 - protein, 11 aa, Mus musculus
  REGION 1..11: note = M P1-G7-LCDR1
RASENIYRNLA

SEQ ID NO: 128 - protein, 9 aa, Mus musculus
  REGION 1..9: note = M P1-G7-LCDR3
QQFWGSPYT

SEQ ID NO: 129 - protein, 5 aa, Mus musculus
  REGION 1..5: note = M P2-C7-HCDR1
DYSMH

SEQ ID NO: 130 - protein, 17 aa, Mus musculus
  REGION 1..17: note = M P2-C7-HCDR2
YFNPNNGDTIYNQKFKG

SEQ ID NO: 131 - protein, 15 aa, Mus musculus
  REGION 1..15: note = M P2-C7-HCDR3
EDSVSTNAWDGALDY

SEQ ID NO: 132 - protein, 11 aa, Mus musculus
  REGION 1..11: note = M P2-C7-LCDR1
RTSENIYSNLA

SEQ ID NO: 133 - protein, 7 aa, Mus musculus
  REGION 1..7: note = M P2-C7-LCDR2
GATNLAD

SEQ ID NO: 134 - protein, 9 aa, Mus musculus
  REGION 1..9: note = M P2-C7-LCDR3
QHFWDIPWT

SEQ ID NO: 135 - protein, 5 aa, Mus musculus
  REGION 1..5: note = M P2-C6-HCDR1
DYYIH

SEQ ID NO: 136 - protein, 17 aa, Mus musculus
  REGION 1..17: note = M P2-C6-HCDR2
YIYPDNGYNGYNQKFKG

SEQ ID NO: 137 - protein, 11 aa, Mus musculus
  REGION 1..11: note = M P2-C6-HCDR3
RGDYSGVHFDY

SEQ ID NO: 138 - protein, 15 aa, Mus musculus
  REGION 1..15: note = M P2-C6-LCDR1
RASESVNNYGVGFLH

SEQ ID NO: 139 - protein, 7 aa, Mus musculus
  REGION 1..7: note = M P2-C6-LCDR2
RASTLES

SEQ ID NO: 140 - protein, 10 aa, Mus musculus
  REGION 1..10: note = M P2-C6-LCDR3
QHSYKDPPWT

SEQ ID NO: 141 - protein, 5 aa, Mus musculus
  REGION 1..5: note = M P1-D7-HCDR1
TYGMN

SEQ ID NO: 142 - protein, 17 aa, Mus musculus
  REGION 1..17: note = M P1-D7-HCDR2
WINTYSGVPTYADDFKG

SEQ ID NO: 143 - protein, 11 aa, Mus musculus
  REGION 1..11: note = M P1-D7-HCDR3
RTDYDGYVMDC

SEQ ID NO: 144 - protein, 17 aa, Mus musculus
  REGION 1..17: note = M P1-D7-LCDR1
KSSQSLFYSGNQKNYLA

SEQ ID NO: 145 - protein, 7 aa, Mus musculus
  REGION 1..7: note = M P1-D7-LCDR2
WASTRES

SEQ ID NO: 146 - protein, 9 aa, Mus musculus
  REGION 1..9: note = M P1-D7-LCDR3
QQYFSYPPT

SEQ ID NO: 147 - protein, 5 aa, Mus musculus
  REGION 1..5: note = M P1-E11-HCDR1
DYSIH

SEQ ID NO: 148 - protein, 17 aa, Mus musculus
  REGION 1..17: note = M P1-E11-HCDR2
YINPNNGVTNYNQKFKG

SEQ ID NO: 149 - protein, 16 aa, Mus musculus
  REGION 1..16: note = M P1-E11-HCDR3
EGDSVTTIVVHWYFDV

SEQ ID NO: 150 - protein, 11 aa, Mus musculus
  REGION 1..11: note = M P1-E11-LCDR1
RASENIYSNLV

SEQ ID NO: 151 - protein, 7 aa, Mus musculus
  REGION 1..7: note = M P1-E11-LCDR2
AAINLVD

SEQ ID NO: 152 - protein, 9 aa, Mus musculus
  REGION 1..9: note = M P1-E11-LCDR3
QQFWDTPLT

SEQ ID NO: 153 - protein, 5 aa, Mus musculus
  REGION 1..5: note = M P2-B9-HCDR1
DYFMN

SEQ ID NO: 154 - protein, 17 aa, Mus musculus
  REGION 1..17: note = M P2-B9-HCDR2
DINPNNGGTSYNQRFKD

SEQ ID NO: 155 - protein, 14 aa, Mus musculus
  REGION 1..14: note = M P2-B9-HCDR3
NGYYDSNYLSYFDV

SEQ ID NO: 156 - protein, 16 aa, Mus musculus
  REGION 1..16: note = M P2-C2-HCDR2
VIWSGGHTDLNAAFIS

SEQ ID NO: 157 - protein, 9 aa, Mus musculus
  REGION 1..9: note = M P2-C2-HCDR3
NGYGGCLDY

SEQ ID NO: 158 - protein, 11 aa, Mus musculus
  REGION 1..11: note = M P2-C2-LCDR1
KAGQDVSIAVA

SEQ ID NO: 159 - protein, 9 aa, Mus musculus
  REGION 1..9: note = M P2-C2-LCDR3
QQYYSIPPS

SEQ ID NO: 160 - protein, 16 aa, Mus musculus
  REGION 1..16: note = M P2-E10-HCDR2
YMSYEGTNNSNPSLKN

SEQ ID NO: 161 - protein, 14 aa, Mus musculus
  REGION 1..14: note = M P2-E10-HCDR3
ERAYYYGTLGAMDY

SEQ ID NO: 162 - protein, 9 aa, Mus musculus
  REGION 1..9: note = M P2-E10-LCDR3
QHFWDTPFT

SEQ ID NO: 163 - protein, 117 aa, synthetic construct
  source 1..117: note = HCVR
  REGION 1..117: note = P2 HCVR
QVQLVQSGAEVKKPGSSVKVSCKASGYTFTSYDISWVRQAPGQGLEWMGGIIPIFGTANYAQKFQGRVTITADESTSTAYMELSSLRSEDTAVYYCTAGIFLATAYWGQGTLVTVSS

SEQ ID NO: 164 - protein, 107 aa, synthetic construct
  source 1..107: note = LCVR
  REGION 1..107: note = P2 LCVR
DIQMTQSPSSMSASVGDRVTISCQASQDINNYLNWYQQRPGQAPKLLIYDASTLETGVPSRFSGSGSGTYFTLTITSLQPADVATYYCQQYENVPITFGQGTRLEIK

SEQ ID NO: 165 - protein, 5 aa, synthetic construct
  source 1..5: note = CDR
  REGION 1..5: note = P2-HCDR1
SYDIS

SEQ ID NO: 166 - protein, 17 aa, synthetic construct
  source 1..17: note = CDR
  REGION 1..17: note = P2-HCDR2
GIIPIFGTANYAQKFQG

SEQ ID NO: 167 - protein, 8 aa, synthetic construct
  source 1..8: note = CDR
  REGION 1..8: note = P2-HCDR3
GIFLATAY

SEQ ID NO: 168 - protein, 11 aa, synthetic construct
  source 1..11: note = CDR
  REGION 1..11: note = P2-LCDR1
QASQDINNYLN

SEQ ID NO: 169 - protein, 7 aa, synthetic construct
  source 1..7: note = CDR
  REGION 1..7: note = P2-LCDR2
DASTLET

SEQ ID NO: 170 - protein, 9 aa, synthetic construct
  source 1..9: note = CDR
  REGION 1..9: note = P2-LCDR3
QQYENVPIT

SEQ ID NO: 171 - protein, 327 aa, Homo sapiens
  REGION 1..327: note = hIgG4-LALA
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 172 - protein, 107 aa, Homo sapiens [sequence omitted]

SEQ ID NO: 173 - protein, 17 aa, synthetic construct
  source 1..17: note = CDR
  REGION 1..17: note = huP2-E3 VH3-HCDR2
DINPNSGGTSYNQKFKG

SEQ ID NO: 174 - protein, 17 aa, synthetic construct
  source 1..17: note = CDR
  REGION 1..17: note = huP2-E3 VH4-HCDR2
DINPNTGGTSYNQKFKG

SEQ ID NO: 175 - protein, 17 aa, synthetic construct
  source 1..17: note = CDR
  REGION 1..17: note = huP2-E3 VH5-HCDR2
DINPNNVGTSYNQKFKG

SEQ ID NO: 176 - protein, 124 aa, synthetic construct
  source 1..124: note = variable region
  REGION 1..124: note = huP2-E3 VH1
EVQLVQSGAEVKKPGASVKVSCKASGYTFTDYYMNWVRQAPGQRLEWMGDINPNNGGTSYNQKFKGRVTITVDKSASTAYMELSSLRSEDTAVYYCARGGVTTVVATYWYFDVWGQGTTVTVSS

SEQ ID NO: 177 - protein, 124 aa, synthetic construct
  source 1..124: note = variable region
  REGION 1..124: note = huP2-E3 VH2
EVQLVQSGAEVKKPGASVKVSCKASGYTFTDYYMNWVRQAPGQSLEWMGDINPNNGGTSYNQKFKGRVTLTVDKSASTAYMELSSLRSEDTAVYYCARGGVTTVVATYWYFDVWGQGTTVTVSS

SEQ ID NO: 178 - protein, 124 aa, synthetic construct
  source 1..124: note = variable region
  REGION 1..124: note = huP2-E3 VH3
EVQLVQSGAEVKKPGASVKVSCKASGYTFTDYYMNWVRQAPGQSLEWMGDINPNSGGTSYNQKFKGRVTLTVDKSASTAYMELSSLRSEDTAVYYCARGGVTTVVATYWYFDVWGQGTTVTVSS

SEQ ID NO: 179 - protein, 124 aa, synthetic construct
  source 1..124: note = variable region
  REGION 1..124: note = huP2-E3 VH4
EVQLVQSGAEVKKPGASVKVSCKASGYTFTDYYMNWVRQAPGQSLEWMGDINPNTGGTSYNQKFKGRVTLTVDKSASTAYMELSSLRSEDTAVYYCARGGVTTVVATYWYFDVWGQGTTVTVSS

SEQ ID NO: 180 - protein, 124 aa, synthetic construct
  source 1..124: note = variable region
  REGION 1..124: note = huP2-E3 VH5
EVQLVQSGAEVKKPGASVKVSCKASGYTFTDYYMNWVRQAPGQSLEWMGDINPNNVGTSYNQKFKGRVTLTVDKSASTAYMELSSLRSEDTAVYYCARGGVTTVVATYWYFDVWGQGTTVTVSS

SEQ ID NO: 181 - protein, 124 aa, synthetic construct
  source 1..124: note = variable region
  REGION 1..124: note = huP2-E3 VH6
EVQLVQSGAEVKKPGASVKVSCKASGYTFTDYYMNWVKQAPGQRLEWMGDINPNSGGTSYNQKFKGRVTITVDKSGSTAYMELSSLRSEDTAVYYCARGGVTTVVATYWYFDVWGQGTTVTVSS

SEQ ID NO: 182 - protein, 107 aa, synthetic construct
  source 1..107: note = variable region
  REGION 1..107: note = huP2-E3 VL1
DIQMTQSPSSLSASVGDRVTITCRASENIYSNLAWYQQKPGKAPKLLVYAATNVADGVPSRFSGSGSGTDFTLTISSLQPEDFATYYCQQFWDTPFTFGQGTKLEIK

SEQ ID NO: 183 - protein, 107 aa, synthetic construct
  source 1..107: note = variable region
  REGION 1..107: note = huP2-E3 VL2
DIQMTQSPSSLSASVGDRVTITCRASENIYSNLAWYQQKPGKSPKLLVYAATNVADGVPSRFSGSGSGTDYTLTISSLQPEDFATYYCQQFWDTPFTFGQGTKLEIK

SEQ ID NO: 184 - protein, 123 aa, synthetic construct
  source 1..123: note = variable region
  REGION 1..123: note = huP2-G9 VH1
EVQLQESGPGLVKPSQTLSLTCTVSGSSITSGYYWNWIRQHPGKGLEWIGYLSYDGSNNYNPSLKNLVTISRDTSKNQFSLKLSSVTAADTAVYYCAREGAHYSGTLSWFAYWGQGTLVTVSS

SEQ ID NO: 185 - protein, 123 aa, synthetic construct
  source 1..123: note = variable region
  REGION 1..123: note = huP2-G9 VH2
EVQLQESGPGLVKPSQTLSLTCTVSGSSITSGYYWNWIRQHPGKKLEWIGYLSYDGSNNYNPSLKNLVTISRDTSKSQFSLKLSSVTAADTAVYYCAREGAHYSGTLSWFAYWGQGTLVTVSS

SEQ ID NO: 186 - protein, 107 aa, synthetic construct
  source 1..107: note = variable region
  REGION 1..107: note = huP2-G9 VL1
DIQMTQSPSSLSASVGDRVTITCRASENIYSNLAWYQQKPGKAPKLLVYAATNLADGVPSRFSGSGSGTDFTLTISSLQPEDFATYYCQHFWDTPYTFGGGTKVEIK

SEQ ID NO: 187 - protein, 107 aa, synthetic construct
  source 1..107: note = variable region
  REGION 1..107: note = huP2-G9 VL2
DIQMTQSPSSLSASVGDRVTITCRASENIYSNLAWYQQKPGKSPKLLVYAATNLADGVPSRFSGSGSGTDYTLTISSLQPEDFATYYCQHFWDTPYTFGGGTKVEIK

SEQ ID NO: 188 - protein, 17 aa, synthetic construct
  source 1..17: note = CDR
  REGION 1..17: note = huP2-E8 VH3-HCDR2
YISSGSNIINYADTVKG

SEQ ID NO: 189 - protein, 17 aa, synthetic construct
  source 1..17: note = CDR
  REGION 1..17: note = huP2-E8 VH4-HCDR2
YISSGSSIHNYADTVKG

SEQ ID NO: 190 - protein, 122 aa, synthetic construct
  source 1..122: note = variable region
  REGION 1..122: note = huP2-E8 VH1
EVQLVESGGGLVKPGGSLRLSCAASGFTFSDYGIHWVRQAPGKGLEWVSYISSGSSIINYADTVKGRFTISRDNAKNSLYLQMNSLRAEDTAVYYCARGPNYYGSSYLLVYWGQGTLVTVSS

SEQ ID NO: 191 - protein, 122 aa, synthetic construct
  source 1..122: note = variable region
  REGION 1..122: note = huP2-E8 VH2
EVQLVESGGGLVKPGGSLRLSCAASGFTFSDYGIHWVRQAPGKGLEWVAYISSGSSIINYADTVKGRFTISRDNAKNSLYLQMNSLRAEDTAVYYCARGPNYYGSSYLLVYWGQGTLVTVSS

SEQ ID NO: 192 - protein, 122 aa, synthetic construct
  source 1..122: note = variable region
  REGION 1..122: note = huP2-E8 VH3
EVQLVESGGGLVKPGGSLRLSCAASGFTFSDYGIHWVRQAPGKGLEWVAYISSGSNIINYADTVKGRFTISRDNAKNSLYLQMNSLRAEDTAVYYCARGPNYYGSSYLLVYWGQGTLVTVSS

SEQ ID NO: 193 - protein, 122 aa, synthetic construct
  source 1..122: note = variable region
  REGION 1..122: note = huP2-E8 VH4
EVQLVESGGGLVKPGGSLRLSCAASGFTFSDYGIHWVRQAPGKGLEWVAYISSGSSIHNYADTVKGRFTISRDNAKNSLYLQMNSLRAEDTAVYYCARGPNYYGSSYLLVYWGQGTLVTVSS

SEQ ID NO: 194 - protein, 107 aa, synthetic construct
  source 1..107: note = variable region
  REGION 1..107: note = huP2-E8 VL1
DIQMTQSPSSLSASVGDRVTITCKASQDVNIAVAWYQQKPGKAPKLLIYSASYRYTGVPSRFSGSGSGTDFTFTISSLQPEDIATYYCQQHYNIPWTFGGGTKVEIK

SEQ ID NO: 195 - protein, 107 aa, synthetic construct
  source 1..107: note = variable region
  REGION 1..107: note = huP2-E8 VL2
DIQMTQSPSSLSASVGDRVTITCKASQDVNIAVAWYQQKPGKSPKLLIYSASYRYTGVPSRFSGSGSGTDFTFTISSLQPEDIAVYYCQQHYNIPWTFGGGTKVEIK

SEQ ID NO: 196 - protein, 107 aa, synthetic construct
  source 1..107: note = variable region
  REGION 1..107: note = huP2-E8 VL3
DIQMTQSPSSLSASVGDRVTITCKASQDVNIAVAWYQQRPGQSPKLLIYSASYRYTGVPSRFSGSGSGTDFTFTISSLQPEDIAVYYCQQHYNIPWTFGGGTKVEIK

SEQ ID NO: 197 - protein, 122 aa, synthetic construct
  source 1..122: note = variable region
  REGION 1..122: note = huP1-G7 VH1
EVQLQESGPGLVKPSQTLSLTCTVSGYSITSGYYWNWIRQHPGKGLEWIGYISYDGGNNYNPFLKNRVTISRDTSKNQFSLKLSSVTAADTAVYYCAREGGSSWEYYGMDYWGQGTTVTVSS

SEQ ID NO: 198 - protein, 122 aa, synthetic construct
  source 1..122: note = variable region
  REGION 1..122: note = huP1-G7 VH2
EVQLQESGPGLVKPSQTLSLTCTVSGYSITSGYYWNWIRQHPEKKLEWIGYISYDGGNNYNPFLKNRVTISRDTSKNQFSLKLSSVTAADTAVYYCAREGGSSWEYYGMDYWGQGTTVTVSS

SEQ ID NO: 199 - protein, 107 aa, synthetic construct
  source 1..107: note = variable region
  REGION 1..107: note = huP1-G7 VL1
DIQMTQSPSSLSASVGDRVTITCRASENIYRNLAWYQQKPGKAPKLLLYAATNLADGVPSRFSGSGSGTDFTLTISSLQPEDFATYYCQQFWGSPYTFGGGTKVEIK

SEQ ID NO: 200 - protein, 107 aa, synthetic construct
  source 1..107: note = variable region
  REGION 1..107: note = huP1-G7 VL2
DIQMTQSPSSLSASVGDRVTITCRASENIYRNLAWYQQKPGKSPKLLLYAATNLADGVPSRFSGSGSGTDYTLTISSLQPEDFATYYCQQFWGSPYTFGGGTKVEIK

SEQ ID NO: 201 - protein, 17 aa, synthetic construct
  source 1..17: note = CDR
  REGION 1..17: note = huP2-C6 VH3-HCDR2
YIYPDNGYSGYNQKFKG

SEQ ID NO: 202 - protein, 17 aa, synthetic construct
  source 1..17: note = CDR
  REGION 1..17: note = huP2-C6 VH4-HCDR2
YIYPDNGYTGYNQKFKG

SEQ ID NO: 203 - protein, 17 aa, synthetic construct
  source 1..17: note = CDR
  REGION 1..17: note = huP2-C6 VH5-HCDR2
YIYPDTGYSGYNQKFKG

SEQ ID NO: 204 - protein, 17 aa, synthetic construct
  source 1..17: note = CDR
  REGION 1..17: note = huP2-C6 VH6-HCDR2
YIYPDSGYNGYNQKFKG

SEQ ID NO: 205 - protein, 120 aa, synthetic construct
  source 1..120: note = variable region
  REGION 1..120: note = huP2-C6 VH1
EVQLVQSGAEVKKPGASVKVSCKASGYTFTDYYIHWVRQAPGQGLEWMGYIYPDNGYNGYNQKFKGRVTMTVDKSTSTAYMELRSLRSDDTAVYYCTRRGDYSGVHFDYWGQGTTVTVSS

SEQ ID NO: 206 - protein, 120 aa, synthetic construct
  source 1..120: note = variable region
  REGION 1..120: note = huP2-C6 VH2
EVQLVQSGAEVKKPGASVKVSCKASGYTFTDYYIHWVRQAPGEGLEWMGYIYPDNGYNGYNQKFKGRVTLTVDKSTNTAYMELRSLRSDDTAVYYCTRRGDYSGVHFDYWGQGTTVTVSS

SEQ ID NO: 207 - protein, 120 aa, synthetic construct
  source 1..120: note = variable region
  REGION 1..120: note = huP2-C6 VH3
EVQLVQSGAEVKKPGASVKVSCKASGYTFTDYYIHWVRQAPGEGLEWMGYIYPDNGYSGYNQKFKGRVTLTVDKSTNTAYMELRSLRSDDTAVYYCTRRGDYSGVHFDYWGQGTTVTVSS

SEQ ID NO: 208 - protein, 120 aa, synthetic construct
  source 1..120: note = variable region
  REGION 1..120: note = huP2-C6 VH4
EVQLVQSGAEVKKPGASVKVSCKASGYTFTDYYIHWVRQAPGEGLEWMGYIYPDNGYTGYNQKFKGRVTLTVDKSTNTAYMELRSLRSDDTAVYYCTRRGDYSGVHFDYWGQGTTVTVSS

SEQ ID NO: 209 - protein, 120 aa, synthetic construct
  source 1..120: note = variable region
  REGION 1..120: note = huP2-C6 VH5
EVQLVQSGAEVKKPGASVKVSCKASGYTFTDYYIHWVRQAPGEGLEWMGYIYPDTGYSGYNQKFKGRVTLTVDKSTNTAYMELRSLRSDDTAVYYCTRRGDYSGVHFDYWGQGTTVTVSS

SEQ ID NO: 210 - protein, 120 aa, synthetic construct
  source 1..120: note = variable region
  REGION 1..120: note = huP2-C6 VH6
EVQLVQSGAEVKKPGASVKVSCKASGYTFTDYYIHWVRQAPGEGLEWMGYIYPDSGYNGYNQKFKGRVTLTVDKSTNTAYMELRSLRSDDTAVYYCTRRGDYSGVHFDYWGQGTTVTVSS

SEQ ID NO: 211 - protein, 120 aa, synthetic construct
  source 1..120: note = variable region
  REGION 1..120: note = huP2-C6 VH7
EVQLVQSGAEVKKPGASVKVSCKASGYTFTDYYIHWVRQAPGQGLEWMGYIYPDTGYSGYNQKFKGRVTMTVDKSTSTAYMELRSLRSDDTAVYYCTRRGDYSGVHFDYWGQGTTVTVSS

SEQ ID NO: 212 - protein, 112 aa, synthetic construct
  source 1..112: note = variable region
  REGION 1..112: note = huP2-C6 VL1
DIVLTQSPDSLAVSLGERATINCRASESVNNYGVGFLHWYQQKPGQPPKLLIYRASTLESGIPDRFSGSGSGTDFTLTISSLQAEDVAVYYCQHSYKDPPWTFGGGTKVEIK

SEQ ID NO: 213 - protein, 112 aa, synthetic construct
  source 1..112: note = variable region
  REGION 1..112: note = huP2-C6 VL2
DIVLTQSPDSLAVSLGERATINCRASESVNNYGVGFLHWYQQKPGQPPKLLIYRASTLESGIPDRFSGSGSRTDFTLTISSLQAEDVATYYCQHSYKDPPWTFGGGTKVEIK

SEQ ID NO: 214 - protein, 112 aa, synthetic construct
  source 1..112: note = variable region
  REGION 1..112: note = huP2-C6 VL3
EIVLTQSPATLSLSPGERATLSCRASESVNNYGVGFLHWYQQKPGQPPKLLIYRASTLESGIPARFSGSGSRTDFTLTISSLEPEDFAVYYCQHSYKDPPWTFGGGTKVEIK

SEQ ID NO: 215 - protein, 17 aa, synthetic construct
  source 1..17: note = CDR
  REGION 1..17: note = huP1-D7 VH1-HCDR2
WINTYSGVPTYAEDFKG

SEQ ID NO: 216 - protein, 11 aa, synthetic construct
  source 1..11: note = CDR
  REGION 1..11: note = huP1-D7 VH1-HCDR3
RTDYDGYVMDS

SEQ ID NO: 217 - protein, 120 aa, synthetic construct
  source 1..120: note = variable region
  REGION 1..120: note = huP1-D7 VH1
EVQLVQSGAEVKKPGASVKVSCKASGYTFTTYGMNWVRQAPGQGLEWMGWINTYSGVPTYAEDFKGRVTMTTDTSTSTAYMELRSLRSDDTAVYYCARRTDYDGYVMDSWGQGTTVTVSS

SEQ ID NO: 218 - protein, 120 aa, synthetic construct
  source 1..120: note = variable region
  REGION 1..120: note = huP1-D7 VH2
EIQLVQSGAEVKKPGASVKVSCKASGYTFTTYGMNWVRQAPGQGLEWMGWINTYSGVPTYAEDFKGRVTMTTDTSTTTAYMELRSLRSDDTAVYYCARRTDYDGYVMDSWGQGTTVTVSS

SEQ ID NO: 219 - protein, 120 aa, synthetic construct
  source 1..120: note = variable region
  REGION 1..120: note = huP1-D7 VH3
EIQLVQSGAEVKKPGASVKVSCKASGYTFTTYGMNWVRQAPGQALKWMGWINTYSGVPTYAEDFKGRVTMTTDTSTTTAYMELRSLRSDDTAVYYCARRTDYDGYVMDSWGQGTTVTVSS

SEQ ID NO: 220 - protein, 120 aa, synthetic construct
  source 1..120: note = variable region
  REGION 1..120: note = huP1-D7 VH4
EIQLVQSGAEVKKPGASVKVSCKASGYTFTTYGMNWVRQAPGQALKWMGWINTYSGVPTYADDFKGRVTMTTDTSTTTAYMELRSLRSDDTAVYYCARRTDYDGYVMDSWGQGTTVTVSS

SEQ ID NO: 221 - protein, 120 aa, synthetic construct
  source 1..120: note = variable region
  REGION 1..120: note = huP1-D7 VH5
EIQLVQSGAEVKKPGASVKVSCKASGYTFTTYGMNWVRQAPGQALKWMGWINTYSGVPTYADDFKGRVTMTTDTSTTTAYMELRSLRSDDTAVYYCARRTDYDGYVMDCWGQGTTVTVSS

SEQ ID NO: 222 - protein, 113 aa, synthetic construct
  source 1..113: note = variable region
  REGION 1..113: note = huP1-D7 VL1
DIVMTQSPDSLAVSLGERATINCKSSQSLFYSGNQKNYLAWYQQKPGQSPKLLLYWASTRESGVPDRFSGSGSGTDFTLTISSLQAEDVAVYYCQQYFSYPPTFGGGTKVEIK

SEQ ID NO: 223 - protein, 113 aa, synthetic construct
  source 1..113: note = variable region
  REGION 1..113: note = huP1-D7 VL2
DIVMTQSPLSLPVTPGEPASISCKSSQSLFYSGNQKNYLAWYLQKPGQSPQLLLYWASTRESGVPDRFSGSGSGTDFTLKISRVEAEDVGVYYCQQYFSYPPTFGGGTKVEIK

SEQ ID NO: 224 - protein, 11 aa, synthetic construct
  source 1..11: note = CDR
  REGION 1..11: note = P2-VL1-LCDR1
QASQDIQNYLN

SEQ ID NO: 225 - protein, 107 aa, synthetic construct
  source 1..107: note = variable region
  REGION 1..107: note = P2m LCVR
DIQMTQSPSSMSASVGDRVTISCQASQDIQNYLNWYQQRPGQAPKLLIYDASTLETGVPSRFSGSGSGTYFTLTISSLQPEDVATYYCQQYENVPITFGQGTRLEIK

SEQ ID NO: 226 - protein, 451 aa, synthetic construct
  source 1..451: note = HC
  CHAIN 1..451: note = huP2-E3-heavy chain
EVQLVQSGAEVKKPGASVKVSCKASGYTFTDYYMNWVKQAPGQRLEWMGDINPNSGGTSYNQKFKGRVTITVDKSGSTAYMELSSLRSEDTAVYYCARGGVTTVVATYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 227 - protein, 214 aa, synthetic construct
  source 1..214: note = LC
  CHAIN 1..214: note = huP2-E3-light chain
DIQMTQSPSSLSASVGDRVTITCRASENIYSNLAWYQQKPGKSPKLLVYAATNVADGVPSRFSGSGSGTDYTLTISSLQPEDFATYYCQQFWDTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 453 aa, synthetic construct
  source 1..453: note = HC
  REGION 1..453: note = huP2-G9-heavy chain
EVQLQESGPGLVKPSQTLSLTCTVSGSSITSGYYWNWIRQHPGKGLEWIGYLSYDGSNNYNPSLKNLVTISRDTSKNQFSLKLSSVTAADTAVYYCAREGAHYSGTLSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 214 aa, synthetic construct
  source 1..214: note = LC
  CHAIN 1..214: note = huP2-G9-light chain
DIQMTQSPSSLSASVGDRVTITCRASENIYSNLAWYQQKPGKSPKLLVYAATNLADGVPSRFSGSGSGTDYTLTISSLQPEDFATYYCQHFWDTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 449 aa, synthetic construct
  source 1..449: note = HC
  CHAIN 1..449: note = huP2-E8-heavy chain
EVQLVESGGGLVKPGGSLRLSCAASGFTFSDYGIHWVRQAPGKGLEWVAYISSGSSIHNYADTVKGRFTISRDNAKNSLYLQMNSLRAEDTAVYYCARGPNYYGSSYLLVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 231 - protein, 214 aa, synthetic construct
  source 1..214: note = LC
  CHAIN 1..214: note = huP2-E8-light chain
DIQMTQSPSSLSASVGDRVTITCKASQDVNIAVAWYQQKPGKAPKLLIYSASYRYTGVPSRFSGSGSGTDFTFTISSLQPEDIATYYCQQHYNIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 449 aa, synthetic construct
  source 1..449: note = HC
  CHAIN 1..449: note = huP1-G7-heavy chain
EVQLQESGPGLVKPSQTLSLTCTVSGYSITSGYYWNWIRQHPGKGLEWIGYISYDGGNNYNPFLKNRVTISRDTSKNQFSLKLSSVTAADTAVYYCAREGGSSWEYYGMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 233 - protein, 214 aa, synthetic construct
  source 1..214: note = LC
  CHAIN 1..214: note = huP1-G7-light chain
DIQMTQSPSSLSASVGDRVTITCRASENIYRNLAWYQQKPGKAPKLLLYAATNLADGVPSRFSGSGSGTDFTLTISSLQPEDFATYYCQQFWGSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - protein, 450 aa, synthetic construct
  source 1..450: note = HC
  CHAIN 1..450: note = huP2-C6-heavy chain
EVQLVQSGAEVKKPGASVKVSCKASGYTFTDYYIHWVRQAPGQGLEWMGYIYPDTGYSGYNQKFKGRVTMTVDKSTSTAYMELRSLRSDDTAVYYCTRRGDYSGVHFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 219 aa, synthetic construct
  source 1..219: note = LC
  CHAIN 1..219: note = huP2-C6-light chain
DIVLTQSPDSLAVSLGERATINCRASESVNNYGVGFLHWYQQKPGQPPKLLIYRASTLESGIPDRFSGSGSGTDFTLTISSLQAEDVAVYYCQHSYKDPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 447 aa, synthetic construct
  source 1..447: note = HC
  CHAIN 1..447: note = huP1-D7-heavy chain
EVQLVQSGAEVKKPGASVKVSCKASGYTFTTYGMNWVRQAPGQGLEWMGWINTYSGVPTYAEDFKGRVTMTTDTSTSTAYMELRSLRSDDTAVYYCARRTDYDGYVMDSWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 237 - protein, 220 aa, synthetic construct
  source 1..220: note = LC
  CHAIN 1..220: note = huP1-D7-light chain
DIVMTQSPDSLAVSLGERATINCKSSQSLFYSGNQKNYLAWYQQKPGQSPKLLLYWASTRESGVPDRFSGSGSGTDFTLTISSLQAEDVAVYYCQQYFSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 447 aa, synthetic construct
  source 1..447: note = HC
  CHAIN 1..447: note = P2m-heavy chain
QVQLVQSGAEVKKPGSSVKVSCKASGYTFTSYDISWVRQAPGQGLEWMGGIIPIFGTANYAQKFQGRVTITADESTSTAYMELSSLRSEDTAVYYCTAGIFLATAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 214 aa, synthetic construct
  source 1..214: note = LC
  CHAIN 1..214: note = P2m-light chain
DIQMTQSPSSMSASVGDRVTISCQASQDIQNYLNWYQQRPGQAPKLLIYDASTLETGVPSRFSGSGSGTYFTLTISSLQPEDVATYYCQQYENVPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 330 aa, Homo sapiens
  REGION 1..330: note = hIgG1-LALA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 448 aa, synthetic construct
  source 1..448: note = HC
  CHAIN 1..448: note = MK-heavy chain
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 242 - protein, 217 aa, synthetic construct
  source 1..217: note = LC
  CHAIN 1..217: note = MK-light chain
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 243 - protein, 450 aa, synthetic construct
  source 1..450: note = HC
  CHAIN 1..450: note = JTX-heavy chain
QITLKESGPTLVKPTQTLTLTCTFSGFSLNTYAMGVSWIRQPPGKALEWLASIWWNGNKYNNPSLKSRLTVTKDTSKNQVVLTMTNMDPVDTATYYCAHSRIIRFTDYVMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 244 - protein, 214 aa, synthetic construct
  source 1..214: note = LC
  CHAIN 1..214: note = JTX-light chain
DIQMTQSPSSLSTSVGDRVTITCRASEDIYNDLAWYQQKPGKAPKLLIYNANSLHTGVASRFSGSGSGTDFTFTISSLQPEDVATYFCQQYYDYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 444 aa, synthetic construct
  source 1..444: note = ILT2-His
  CHAIN 1..444: note = ILT2-His
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVHHHHHH

SEQ ID NO: 246 - protein, 244 aa, synthetic construct
  source 1..244: note = ILT3-His
  CHAIN 1..244: note = ILT3-His
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWEHHHHHH

SEQ ID NO: 247 - protein, 338 aa, Homo sapiens
  CHAIN 1..338: note = HLA-G
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 248 - protein, 452 aa, synthetic construct
  source 1..452: note = HC
  CHAIN 1..452: note = Hu23-11.IgG4AA
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 249 - protein, 219 aa, synthetic construct
  source 1..219: note = LC
  CHAIN 1..219: note = Hu23-11-light chain
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 5 aa, synthetic construct
  source 1..5: note = CDR
  REGION 1..5: note = PD-1-HCDR1
DYEMH

SEQ ID NO: 251 - protein, 17 aa, synthetic construct
  source 1..17: note = CDR
  REGION 1..17: note = PD-1-HCDR2
LIDPETGGTVYNQKFKD

SEQ ID NO: 252 - protein, 16 aa, synthetic construct
  source 1..16: note = CDR
  CHAIN 1..16: note = PD-1-HCDR3
ERFSYYGSTSDWYFDV

SEQ ID NO: 253 - protein, 16 aa, synthetic construct
  source 1..16: note = CDR
  REGION 1..16: note = PD-1-LCDR1
RSSQSLVHSTGNTYLE

SEQ ID NO: 254 - protein, 7 aa, synthetic construct
  source 1..7: note = CDR
  CHAIN 1..7: note = PD-1-LCDR2
KVSNRFS

SEQ ID NO: 255 - protein, 9 aa, synthetic construct
  source 1..9: note = CDR
  REGION 1..9: note = PD-1-LCDR3
FQGSHVPYT

SEQ ID NO: 256 - protein, 125 aa, synthetic construct
  source 1..125: note = VH
  REGION 1..125: note = PD-1 VH
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSS

SEQ ID NO: 257 - protein, 112 aa, synthetic construct
  source 1..112: note = VL
  REGION 1..112: note = PD-1 VL
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIK

SEQ ID NO: 258 - protein, 448 aa, synthetic construct
  source 1..448: note = HC
  CHAIN 1..448: note = anti PD-1-heavy chain
EVRLLESGGGLVKPEGSLKLSCVASGFTFSDYFMSWVRQAPGKGLEWVAHIYTKSYNYATYYSGSVKGRFTISRDDSRSMVYLQMNNLRTEDTATYYCTRDGSGYPSLDFWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 259 - protein, 216 aa, synthetic construct
  source 1..216: note = LC
  CHAIN 1..216: note = anti PD-1-light chain
YELTQPPSASVNVGETVKITCSGDQLPKYFADWFHQRSDQTILQVIYDDNKRPSGIPERISGSSSGTTATLTIRDVRAEDEGDYYCFSGYVDSDSKLYVFGSGTQLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 9 aa, Mus musculus
  DOMAIN 1..9: note = huP2-G9-HCDR1-IMGT
GSSITSGYY

SEQ ID NO: 261 - protein, 7 aa, Mus musculus
  DOMAIN 1..7: note = huP2-G9-HCDR2-IMGT
LSYDGSN

SEQ ID NO: 262 - protein, 16 aa, Mus musculus
  DOMAIN 1..16: note = huP2-G9-HCDR3-IMGT
AREGAHYSGTLSWFAY

SEQ ID NO: 263 - protein, 6 aa, Mus musculus
  DOMAIN 1..6: note = huP2-G9-LCDR1-IMGT
ENIYSN

SEQ ID NO: 264 - skipped

SEQ ID NO: 265 - protein, 8 aa, Mus musculus
  DOMAIN 1..8: note = huP2-G9-HCDR1-Chothia
GSSITSGY

SEQ ID NO: 266 - protein, 5 aa, Mus musculus
  DOMAIN 1..5: note = huP2-G9-HCDR2-Chothia
SYDGS

SEQ ID NO: 267 - protein, 8 aa, Mus musculus
  DOMAIN 1..8: note = huP2-C6-HCDR1-IMGT
GYTFTDYY

SEQ ID NO: 268 - protein, 8 aa, synthetic construct
  DOMAIN 1..8: note = huP2-C6-HCDR2-IMGT
IYPDTGYS

SEQ ID NO: 269 - protein, 13 aa, Mus musculus
  DOMAIN 1..13: note = huP2-C6-HCDR3-IMGT
TRRGDYSGVHFDY

SEQ ID NO: 270 - protein, 10 aa, Mus musculus
  DOMAIN 1..10: note = huP2-C6-LCDR1-IMGT
ESVNNYGVGF

SEQ ID NO: 271 - skipped

SEQ ID NO: 272 - protein, 7 aa, Mus musculus
  DOMAIN 1..7: note = huP2-C6-HCDR1-Chothia
GYTFTDY

SEQ ID NO: 273 - protein, 6 aa, synthetic construct
  DOMAIN 1..6: note = huP2-C6-HCDR2-Chothia
YPDTGY